(19) United States
(12) Patent Application Publication
(10) Pub. No.: US 2025/0090583 A1
(43) Pub. Date: Mar. 20, 2025

(54) UNIVERSAL TCR VARIANTS FOR ALLOGENEIC IMMUNOTHERAPY

(71) Applicant: ETH ZURICH, Zurich (CH)

(72) Inventors: Edo Kapetanovic, Basel (CH); Sai Tota Reddy, Basel (CH); Cédric Ramon Weber, Zürich (CH); Rodrigo Vazquez-Lombardi, Riehen (CH)

(73) Assignee: ETH ZURICH, Zurich (CH)

(21) Appl. No.: 18/727,881

(22) PCT Filed: Jan. 24, 2023
(86) PCT No.: PCT/EP2023/051688

§ 371 Date: Jul. 10, 2024

(30) Foreign Application Priority Data
Jan. 24, 2022 (EP) 22153056.1

(51) Int. Cl. A61K 35/17; A61K 39/00; A61P 35/00; C07K 14/725; C12N 5/0783

(52) U.S. Cl. CPC A61K 35/17; A61K 40/11; A61K 40/32; A61P 35/00; C07K 14/7051; C12N 5/0636; C12N 2510/00

(57) ABSTRACT

The present invention relates to a T cell comprising an engineered TCR-CD3 complex, wherein (a) binding of the engineered TCR-CD3 complex by a CD3 agonist results in a similar level of T cell activation compared to a T cell comprising a non-engineered TCR-CD3 complex; and (b) binding of the engineered TCR-CD3 complex by a cognate peptide-MHC complex results in a reduced level of T cell activation compared to a T cell comprising a non-engineered TCR-CD3 complex. Further encompassed are compositions comprising the T cell according to the invention and methods of uses thereof.

Several cancer immunotherapies rely on the functional activation of endogenous host T cells. For example, T cell engaging bispecific antibodies (biAbs) simultaneously target the CD3 receptors on T cells and a tumor antigen target, thus acting as molecular bridges to induce cytotoxic T cell responses against tumor cells. A prominent example is blinatumomab, a clinically approved biAb used for treatment of hematological malignancies (e.g B cell acute lymphoblastic leukemias, B-ALL), which simultaneously binds to CD3 (T cells) and CD19 (B cell tumor cells), and has been successful in prolonging survival in patients with relapsed or refractory B-ALL. However, in blinatumomab-treated patients, it is common to observe relapse that is not driven by tumor antigen escape (CD19+ tumor cells are still present). While the etiology of blinatumomab clinical failure is an active area of investigation, the immunocompromised status of lymphoma patients likely plays a major role due to a depleted fraction of functional autologous T cells.

In order to boost anti-tumor immunity, allogeneic T cells are actively being investigated in the field of immunotherapy (e.g. chimeric antigen receptor (CAR) T cells) due to their promise of ease of manufacturing, scalability and therapeutic consistency. Additionally, combination immunotherapies such as adoptive T cell transfer (e.g. CAR-T, TCR-T cells and tumor infiltrating T lymphocytes) combined with antibodies targeting immunomodulatory receptors (e.g. PD-1 and CTLA-4) are becoming a new standard in immunotherapy. Similarly, introduction of allogeneic T cells in order to augment the immune system of immunocompromised cancer patients would enhance clinical efficacy of biAb. However, such an allogeneic T cell therapy comes with several risks, most notably HLA (MHC) mismatch could result in activation of donor T cells against recipient's self-antigens and lead to adverse and severe onset of graft-versus-host disease (GvHD).

Activation of T cells is primarily regulated through the TCR-CD3 complex, which is a mechanosensory unit composed of the αβ-TCR dimer and CD3γε-CD3δε-CD3ζζ hexamer. The αβ-TCR dimer is responsible for peptide-HLA recognition and transfer of mechanical forces to the non-covalently bound CD3 molecules. TCR-binding to antigen (peptide-HLA) causes conformational changes in the receptor that are transduced via CD3-signaling apparatus to intracellular proteins (e.g. Lck and Zap-70) that activate a plethora of T-cell pathways necessary for T-cell survival, proliferation, differentiation and effector functions. The eight subunits of the TCR-CD3 complex are all indispensable for a functional and stable surface expression. Therefore, in the context of augmenting biAbs with allogeneic T cell transfer, it would be infeasible to simply knockout TCR chains, as misfolding or absence of any of the subunits will result in a complete loss of the TCR-CD3 complex and, consequently, the associated T cell functions necessary for the biAb activation.

Accordingly, there is a need in the art for engineered T cells for use in T cell therapy. In particular, there is a need in the art for engineered T cells for use in allogenic T cell therapy. Further, there is a need in the art for increasing the therapeutic efficacy of T cell engaging bispecific antibodies.

### SUMMARY OF THE INVENTION

The present invention is characterized in the herein provided embodiments and claims. In particular, the present invention relates, inter alia, to the following embodiments:
1. A T cell comprising an engineered TCR-CD3 complex, wherein
   a) binding of the engineered TCR-CD3 complex by a CD3 agonist results in a similar level of T cell activation compared to a T cell comprising a non-engineered TCR-CD3 complex; and
   b) binding of the engineered TCR-CD3 complex by a cognate peptide-MHC complex results in a reduced level of T cell activation compared to a T cell comprising a non-engineered TCR-CD3 complex.
2. The T cell according to embodiment 1, wherein the engineered TCR-CD3 complex comprises at least one mutation in a TCR alpha and/or beta chain.
3. The T cell according to embodiment 2, wherein the at least one mutation in the TCR alpha and/or beta chain is located outside of a complementary determining region (CDR).
4. The T cell according to embodiment 2 or 3, wherein at least one mutation in the TCR alpha and/or beta chain is located at the interface between the TCR alpha and beta chain.
5. The T cell according to any one of embodiments 2-4, wherein the at least one mutation in the TCR alpha and/or beta chain results in reduced TCR alpha and beta association.
6. The T cell according to any one of embodiments 2-5, wherein the at least one mutation in the TCR alpha and/or beta chain is located in the sequence motif WYRQ (IMGT position 41-44), FG₁xG₂T (IMGT position 118-122), VxP (IMGT position 126-128), PDP (position 4-6 of TCR alpha constant region (SEQ ID NO:25)), TDFDS (position 24-29 of TCR alpha constant region (SEQ ID NO:25)) and/or FETDxNLN (position 103-110 of TCR alpha constant region (SEQ ID NO:25)), wherein x is an undefined amino acid.
7. The T cell according to embodiment 6, wherein at least one of the amino acid residues G₁ and/or G₂ in the motif FG₁xG₂T (IMGT position 118-122) has been replaced with another amino acid.
8. The T cell according to embodiment 6 or 7, wherein the motif FG₁xG₂T in the TCR alpha and/or beta chain has been replaced with the sequence FEQWT.
9. The T cell according to any one of embodiments 2-8, wherein the at least one mutation in the engineered TCR-CD3 complex is located:
   a) at position W41, Y/F42, R/Q43, Q44, F118, G119, G121, T/S122, V126 and/or P128 in the variable domain of the TCR alpha chain (according to IMGT numbering); and/or
   b) at position W41, Y/F42, R/Q43, Q44, F118, G119, G121, T/S122, T125 and/or V/L/T127 in the variable domain of the TCR beta chain (according to IMGT numbering); and/or
   c) at position P4, D5, P6, F24, T25, D26, F27, D28, S29, F103, E104, T105, D106, N108, L109, N110 in the TCR alpha constant region (SEQ ID NO:25).
10. The T cell according to any one of embodiments 2-9, wherein the T cell is a human T cell.
11. The T cell according to any one of embodiments 2-10, wherein the TCR alpha and/or beta chain further comprise an affinity tag.
12. The T cell according to embodiment 11, wherein the affinity tag is inserted between the signal peptide and the coding sequence of the TCR alpha and/or beta chain.
13. The T cell according to any one of embodiments 1-12, wherein the CD3 agonist is an anti-CD3 antibody or a fragment thereof.
14. The T cell according to embodiment 13, wherein the anti-CD3 antibody is a bispecific antibody.
15. The T cell according to embodiment 14, wherein the bispecific antibody is blinatumomab.
16. The T cell according to embodiment 13, wherein the anti-CD3 antibody fragment is comprised in a fusion protein.
17. The T cell according to embodiment 16, wherein the fusion protein further comprises a fragment of a T cell receptor.
18. The T cell according to any one of embodiments 1-17, wherein the level of T cell activation is characterized by:
   a) the secretion of interleukin-2 (IL-2); and/or
   b) the secretion of interferon gamma (IFN-v); and/or
   c) the rate of T cell proliferation.
19. A T cell population comprising a plurality of T cells according to any one of embodiments 1-18.
20. A pharmaceutical composition comprising the T cell according to any one of embodiments 1-18 or a T cell population according to embodiment 19.
21. The pharmaceutical composition according to embodiment 20 further comprising a CD3 agonist.
22. The T cell according to any one of embodiments 1-18, the T cell population according to embodiment 19 or the pharmaceutical composition according to embodiment 20 or 21 for use as a medicament.
23. The T cell according to any one of embodiments 1-18, the T cell population according to embodiment 19 or the pharmaceutical composition according to embodiment 20 or 21 for use in the treatment of cancer.
24. The T cell according to any one of embodiments 1-18, the T cell population according to embodiment 19 or the pharmaceutical composition according to embodiment 20 or 21 for use in the treatment of a viral infection.
25. The T cell according to any one of embodiments 1-18, the T cell population according to embodiment 19 or the pharmaceutical composition according to embodiment 20 or 21 for use in the treatment of an autoimmune disease.
26. The T cell, the T cell population or the pharmaceutical composition for use according to any one of embodiments 22-25, wherein the human T cell has been obtained from a patient to be treated.
27. The T cell, the T cell population or the pharmaceutical composition for use according to any one of embodiments 22-25, wherein the human T cell has been obtained from a donor.
28. The T cell, the T cell population or the pharmaceutical composition for use according to any one of embodiments 22-27, wherein the human T cell is administered before, concomitantly or after a CD3 agonist.
29. A method for generating a T cell according to any one of embodiments 1-18, the method comprising the steps of:
   i) introducing at least one mutation into a TCR alpha and/or beta chain of a T cell that has been obtained from a donor;
   ii) obtaining a T cell comprising and engineered TCR-CD3 complex.
30. The method according to embodiment 29, wherein the at least one mutation is introduced into the TCR alpha and/or beta chain by genome editing.
31. The method according to embodiment 30, wherein the genome editing step involves the use of a CRISPR-Cas system.
32. The method according to any one of embodiments 29-31 further comprising a step of introducing a nucleic acid encoding an affinity tag into the TCR alpha and/or beta chain of the T cell.

That is, the invention is, at least in parts, based on the surprising finding that certain mutations in T cell receptors (TCRs) decouple TCR-antigen binding from CD3 signalling. The engineered TCRs of the present invention are still able to bind to their cognate peptide-MHC complex but cannot transform TCR-antigen binding into a CD3 activation signal. Most importantly, the engineered TCRs of the present invention maintain functional TCR-CD3 cell surface expression. As such, T cells comprising the engineered TCR of the invention can be activated by CD3 agonists. In consequence, T cells comprising the engineered TCR of the invention are particularly well suited for use in allogenic T cell therapy, as they reduce the risk of unwanted antigen-induced immune reactions. At the same time, activation of T cells comprising the engineered TCR of the invention can be tightly controlled by administrating a CD3 agonist.

Accordingly, in a particular embodiment, the invention relates to a T cell comprising an engineered TCR-CD3 complex, wherein (a) binding of the engineered TCR-CD3 complex by a CD3 agonist results in a similar level of T cell activation compared to a T cell comprising a non-engineered TCR-CD3 complex; and (b) binding of the engineered TCR-CD3 complex by a cognate peptide-MHC complex results in a reduced level of T cell activation compared to a T cell comprising a non-engineered TCR-CD3 complex.

That is, the invention, in certain embodiments, relates to a T cell comprising an engineered TCR-CD3 complex. In particular, the engineered TCR-CD3 complex comprises one or more mutations in the TCR portion. TCR mutation that result in the engineered TCR-CD3 complex of the invention are disclosed in more detail herein.

The T cell of the present invention is characterized by the presence of an engineered TCR-CD3 complex. A “TCR-CD3 complex”, as used herein, refers to a complex on the cell surface comprising a TCR heterodimer (either αβ or γδ) and the clonally invariant CD3 chains δ, ε, γ and ζ. The engineered TCR-CD3 complex comprises at least one mutation in a nucleic acid encoding a TCR chain, wherein the at least one mutation in a nucleic acid encoding a TCR chain does not significantly affect surface expression of the TCR-CD3 complex. Instead, it is preferred herein that the engineered TCR-CD3 complex of the invention is efficiently displayed on the surface of the T cell.

A protein is said to be expressed on the cell surface of a cell if the protein is embedded in or spans the cell membrane and comprises an extracellular domain that is exposed to the external side of the membrane. The skilled person is aware of methods to determine whether a T cell expresses a TCR-CD3 complex on the cell surface. For example, the presence of a TCR-CD3 complex on the cell surface may be determined by flow cytometry using one or more antibodies that specifically bind to a TCR chain or a CD3 subunit. Quantification of TCR-CD3 complexes on the cell surface has been described, for example, by Isomäki et al. (J Immunol, 2001, 166:5495-5507).

The engineered TCR-CD3 complex of the invention is efficiently displayed on the surface of a T cell. Even though it is preferred that the engineered TCR-CD3 complex is displayed on the surface of a T cell with the same efficiency as a non-engineered TCR-CD3 complex, it is to be understood that introducing one or more mutations in a TCR chain may reduce the level of surface expression of the engineered TCR-CD3 complex to some extent. Thus, in certain embodiments, the engineered TCR-CD3 complex is determined to be efficiently expressed on the surface of a T cell, if the one or more modification in the TCR chain reduce the level of surface expression by not more than 10%, 20%, 30%, 40%, 50%, 60%, 70% or 80% when compared to its non-engineered counterpart. In a preferred embodiment, the engineered TCR-CD3 complex is determined to be efficiently expressed on the surface of a T cell, if the one or more modification in the TCR chain reduce the level of surface expression by not more than 50% when compared to its non-engineered counterpart. In certain embodiments, an engineered TCR-CD3 complex may be determined to be efficiently expressed on the surface of a T cell if the TCR-CD3 complex is detectable by methods known in the art, such as flow cytometry.

It is known in the art that TCR chains comprise a constant domain and a variable domain, wherein the variable domain is the result of a series of recombination events. Despite the sequence variation in the variable domain, the inventors identified conserved sequence motifs that, when mutated, result in decoupling of the TCR. However, due to the higher degree of structural variance in the variable domain, a specific mutation will not necessarily have the same effect in any TCR. For that reason, it is preferred that T cells comprising the engineered TCR-CD3 complex of the invention are defined according to their function.

That is, a T cell comprising an engineered TCR-CD3 complex is preferably characterized by:
(a) a similar level of T cell activation when contacted with a CD3 agonist in comparison to a T cell comprising a non-engineered TCR-CD3 complex; and
(b) a reduced level of T cell activation when contacted with a cognate peptide-MHC complex in comparison to a T cell comprising a non-engineered TCR-CD3 complex.

That is, a T cell comprising an engineered TCR-CD3 complex according to the invention may be identified by comparing the level of T cell activation between a T cell comprising the engineered TCR-CD3 complex and a T cell comprising the non-engineered counterpart of said engineered TCR-CD3 complex under specific conditions.

An “engineered TCR-CD3 complex” as used herein is a protein complex formed by a TCR and a CD3 hexamer, wherein the TCR portion comprises at least one genetic modification. Preferably, the genetic modification in the TCR portion is a mutation in the TCR alpha or beta chain. The “non-engineered counterpart” is a TCR-CD3 complex that differs from the engineered TCR-CD3 complex by a limited number of mutations in the TCR portion, preferably in one or more of the sequence motifs disclosed herein. In certain embodiments, a “non-engineered counterpart” differs from an engineered TCR-CD3 complex of the invention by not more than 1, 2, 3, 4, 5 or 6 amino acid changes, preferably in one or more of the conserved sequence motifs disclosed herein. In certain embodiments, the “non-engineered counterpart” is a TCR-CD3 complex that served as template for an engineered TCR-CD3 complex.

The non-engineered counterpart may be a TCR-CD3 complex comprising a naturally occurring TCR, i.e., a TCR that resulted from natural V (D) J recombination. However, in certain embodiments, the “non-engineered counterpart” may comprise a TCR that has been previously engineered. TCR-CD3 complexes comprising a previously engineered TCR may serve as “non-engineered” counterparts of TCR-CD3 complexes that comprise one or more additional mutations, preferably in one of the sequence motifs disclosed herein. An example is the engineered MAGE-A3-specific TCR a3a, which has been further engineered herein to decouple the TCR-antigen binding from the generation of a CD3 activation signal.

The term “T cell activation”, as used herein, refers to one or more cellular response of a T lymphocyte selected from: proliferation, differentiation, cytokine secretion, cytotoxic effector molecule release, cytotoxic activity, and expression of activation markers. In particular, the term “T cell activation” is used herein to define a state in which a T cell response has been initiated or activated by a primary signal, such as through the TCR-CD3 complex. A T cell is activated if it has received a primary signaling event that initiates an immune response by the T cell. The level of T cell activation may be determined by quantifying a particular immune response.

To characterize the T cell according to the invention, the level of T cell activation is preferably determined by quantifying
a) the secretion of interleukin-2 (IL-2); and/or
b) the secretion of interferon gamma (IFNγ); and/or
c) the rate of T cell proliferation.

Thus, in a particular embodiment, the invention relates to the T cell according to the invention, wherein the level of T cell activation is characterized by:
a) secretion of interleukin-2 (IL-2); and/or
b) secretion of interferon gamma (IFN-γ); and/or
c) the rate of T cell proliferation

IL-2 is a cytokine that is secreted by activated CD4+ and CD8+ T cells. Thus, IL-2 is a suitable activation marker for these types of T cells. Within the present invention, it is assumed that the secretion of IL-2 correlates with T cell activity. The skilled person is aware of methods to quantify the secretion of IL-2 from T cells. Preferably, the secretion of IL-2 is quantified by measuring the concentration of IL-2 in a cell culture supernatant. Various kits are known for the quantification of IL-2 and are commercially available. Further, the skilled person is aware of cell culture media and conditions that are suitable for the culturing of T cells. When comparing the secretion of IL-2 between two cell cultures, e.g., a first culture of T cells comprising an engineered TCR-CD3 complex and a second culture of T cells comprising the non-engineered counterpart of the engineered TCR-CD3 complex, it is preferred that the conditions (cell type, antigen, culture volume, culturing conditions, etc.) in both cell cultures are comparable.

In certain embodiments, a T cell comprising an engineered TCR-CD3 complex secretes similar levels of IL-2 when contacted with a CD3 agonist as a T cell comprising the non-engineered counterpart of the engineered TCR-CD3 complex. A T cell comprising an engineered TCR-CD3 complex is defined to secrete similar levels of IL-2 compared to a T cell comprising the non-engineered counterpart of the engineered TCR-CD3 complex, if the T cell comprising the engineered TCR-CD3 complex secretes at least 50%, 60%, 70%, 80%, 90% of the IL-2 that is secreted by a T cell comprising the non-engineered counterpart of the engineered TCR-CD3 complex in response to a stimulus by a CD3 agonist.

In certain embodiments, a T cell comprising an engineered TCR-CD3 complex secretes lower levels of IL-2 when contacted with a cognate peptide-MHC complex as a T cell comprising the non-engineered counterpart of the engineered TCR-CD3 complex. A T cell comprising an engineered TCR-CD3 complex is defined to secrete lower levels of IL-2 compared to a T cell comprising the non-engineered counterpart of the engineered TCR-CD3 complex, if the T cell comprising the engineered TCR-CD3 complex secretes less than 50%, 40%, 30%, 20%, 10% of the IL-2 that is secreted by a T cell comprising the non-engineered counterpart of the engineered TCR-CD3 complex in response to a stimulus by a cognate MHC-peptide complex. In certain embodiments, a T cell comprising an engineered TCR-CD3 complex is unable to secrete IL-2 upon stimulation with a cognate peptide-MHC complex.

IFNγ is a cytokine that is secreted by activated CD4+ and CD8+ T cells. That is, IFNγ is a suitable activation marker for these types of T cells. Within the present invention, it is assumed that the secretion of IFNγ correlates with T cell activity. The skilled person is aware of methods to quantify the secretion of IFNγ from T cells. Preferably, the secretion of IFNγ is quantified by measuring the concentration of IFNγ in a cell culture supernatant. Various kits are known for the quantification of IFNγ and are commercially available. Further, the skilled person is aware of cell culture media and conditions that are suitable for the culturing of T cells. When comparing the secretion of IFNγ between two cell cultures, e.g., a first culture of T cells comprising an engineered TCR-CD3 complex and a second culture of T cells comprising the non-engineered counterpart of the TCR-CD3 complex, it is preferred that the conditions (cell type, antigen, culture volume, culturing conditions, etc.) in both cell cultures are comparable.

In certain embodiments, a T cell comprising an engineered TCR-CD3 complex secretes similar levels of IFNγ when contacted with a CD3 agonist as a T cell comprising the non-engineered counterpart of the engineered TCR-CD3 complex. A T cell comprising an engineered TCR-CD3 complex is defined to secrete similar levels of IFNγ compared to a T cell comprising the non-engineered counterpart of the engineered TCR-CD3 complex, if the T cell comprising the engineered TCR-CD3 complex secretes at least 50%, 60%, 70%, 80%, 90% of the IFNγ that is secreted by a T cell comprising the non-engineered counterpart of the engineered TCR-CD3 complex in response to a stimulus by a CD3 agonist.

In certain embodiments, a T cell comprising an engineered TCR-CD3 complex secretes lower levels of IFNγ when contacted with a cognate peptide-MHC complex as a T cell comprising the non-engineered counterpart of the engineered TCR-CD3 complex. A T cell comprising an engineered TCR-CD3 complex is defined to secrete lower levels of IFNγ compared to a T cell comprising the non-engineered counterpart of the engineered TCR-CD3 complex, if the T cell comprising the engineered TCR-CD3 complex secretes less than 50%, 40%, 30%, 20%, 10% of the IFNγ that is secreted by a T cell comprising the non-engineered counterpart of the engineered TCR-CD3 complex in response to a stimulus by a cognate MHC-peptide complex. In certain embodiments, a T cell comprising an engineered TCR-CD3 complex is unable to secrete IFNγ upon stimulation with a cognate peptide-MHC complex.

In particular, the secretion of cytokines, such as IL-2 and/or IFNγ, by a T cell may be quantified as follows:

T cells comprising an engineered TCR-CD3 complex and T cells comprising the non-engineered counterpart of the TCR-CD3 complex may be sorted by FACS into separate cultures. Subsequent to sorting, cells may be supplemented with IL-2 and rested. Preferably, cells are rested for 3 days. After the resting period, cells may be washed, counted and resuspended in full T cell media.

The secretion of cytokines in response to stimulation with a cognate peptide-MHC complex is preferably determined in a co-culture assay. For that, T cells are mixed with antigen-presenting cells at a determined ratio. In certain embodiments, the antigen-presenting cell may be a cell naturally presenting the antigen on a matching HLA background. In certain embodiments, the antigen-presenting cell may be a T2 cell that has been pulsed with a peptide. Preferably, the T cells may be mixed with T2 cells that have been pulsed with different concentrations of a peptide. For that, peptides may be generated by custom peptide synthesis (Genscript), re-suspended at 10 mg/mL in DMSO and placed at −80° C. for prolonged storage. For peptide pulsing, T2 cells may be harvested and washed twice in serum-free RPMI 1640 (SF-RPMI). Peptides may be diluted to 1 ng/ml to 10 g/mL in SF-RPMI and the solution may be used to make 10-fold dilutions in which cells are resuspended at a concentration 1×10⁶ cells/mL. Cells may be incubated for 120 min at 37° C., 5% CO2, washed twice with SF-RPMI, resuspended in complete media and added to co-culture wells. In a preferred embodiment, T cells and antigen-presenting cells are mixed at a 1:10 ratio. In an even more preferred embodiment, 5×10³ T cells and 5×10⁴ antigen-presenting cells are mixed in a total volume of 150 μL. The mixed cells may be incubated overnight at 37° C., 5% CO₂. The next day, the cells may be spun down to collect the supernatant. The sedimented cells may be resuspended in fresh media and cultured for an additional period of time, preferably for 4 days. After the additional culturing period, supernatants may be collected again and the remaining cells may be assessed by flow cytometry. Concentration of cytokines may be determined in the collected supernatants using commercial ELISA kits. For example, the concentration of human IL-2 and IFN-γ cytokines may be quantified using standard kits (Thermo Fisher, #88-7025-88 and #88-7316-88). Supernatants may be diluted with media to fall within the standard curve of the assay. Negative control values may be subtracted from each sample point and the concentration may be calculated from the standard curves. Measured concentrations of cytokine may be plotted versus the peptide concentration and fitted to a 4-parameter logistic model.

When quantifying the secretion of cytokines by T cells in response to stimulation with a CD3 agonist, cells may be prepared similarly as described above for the co culture assay. However, instead of mixing the T cells with an antigen-presenting cells, T cells may be contacted with a CD3 agonist. For example, T cells may be prepared as described above and mixed with a CD3 agonist at a suitable concentration. The skilled person is able to determine a concentration of a CD3 agonist that is sufficient to activate a cell. When the CD3 agonist is blinatumomab, 106 T cells may be mixed with 12 ng/mL blinatumomab to induce cytokine secretion in the T cells.

The term “secretion” as used herein refers to translocation of a polypeptide or protein, specifically a cytokine, across the cell membrane. The secreted protein may be either part of the cell membrane as a membrane-bound protein that is anchored within the cell membrane, or released as soluble protein to the cell supernatant.

The skilled person is aware of other cytokines and proteins that are secreted by activated T cells. The skilled person is further aware of the different types of T cells and the secreted cytokines/proteins that are characteristic for these T cells. Thus, secretion of other cytokines and/or proteins may be similarly used to determine the level of T cell activation.

CD8+ T cells typically secrete IFNγ, TNFα, granzymes and perforin upon activation. However, subsets of CD8+ T cell have been identified that secrete different cytokine patterns as shown in the table below:

| CD8+ T cell | |
|---|---|
| phenotype | Secreted cytokines |
| Tc1 | IFNγ, TNFα, granzymes, perforin |
| Tc2 | IL-5, IL-13, IL-4, granzymes, perforin |
| Tc9 | IL-9, IL-10 |
| Tc17 | IL-17, IL-21 |
| CD8+ Treg | TGFβ, IL-10, granzymes, perforin |

CD4+ T cells (T helper cells) are more diverse and may secrete different cytokine patterns, as shown in the table below:

| CD4+ T cell | |
|---|---|
| phenotype | Secreted cytokines |
| Th1 | IL-2, IFNγ, TNFα, TNFβ |
| Th2 | IL-1, IL-3, IL-4, IL-5, IL-6, IL-10, IL-13, GM-CSF |
| Th9 | IL-9 |
| Th17 | IL-17A, IL-17F, IL-17AF, IL-21, IL-22, IL-24, IL-26 |
| Th22 | IL-22, TNFα |
| Treg | TGFβ, IL-10, IL-35 |
| Tfh | IL-6, IL-10 |
| NKT | IL-4, IFNγ, IL-17 |

As can be derived from the tables above, various cytokines/proteins may be used to identify a T cell according to the invention. In this case, a T cell comprising an engineered TCR-CD3 complex is defined to secrete similar levels of a cytokine/protein compared to a T cell comprising the non-engineered counterpart of the engineered TCR-CD3 complex, if the T cell comprising the engineered TCR-CD3 complex secretes at least 50%, 60%, 70%, 80%, 90% of the cytokine/protein that is secreted by a T cell comprising the non-engineered counterpart of the engineered TCR-CD3 complex in response to a stimulus by a CD3 agonist. Similarly, a T cell comprising an engineered TCR-CD3 complex is defined to secrete lower levels of a cytokine/protein compared to a T cell comprising the non-engineered counterpart of the engineered TCR-CD3 complex, if the T cell comprising the engineered TCR-CD3 complex secretes less than 50%, 40%, 30%, 20%, 10% of the cytokine/protein that is secreted by a T cell comprising the non-engineered counterpart of the engineered TCR-CD3 complex in response to a stimulus by a cognate MHC-peptide complex.

However, it is preferred herein that the T cell is a Tc1 or Th1 T cell that can be identified based on the secretion of IL-2 and/or IFNγ.

Activation of T cells by a cognate peptide-MHC complex or other T cell activating agents typically results in proliferation of said T cells. The term “proliferation” as used herein, means to grow or multiply by producing new cells. Within the present invention, it is assumed that the level of T cell proliferation upon stimulation with a cognate antigen or another activating agent correlates with T cell activity. The skilled person is aware of methods to quantify the proliferation of T cells. For example, T cell proliferation is quantified by staining T cells with a fluorescent tracking dye and by monitoring the dilution of the dye in daughter cells as cells get activated and divide over time. Various kits are known for the quantification of T cell proliferation and are commercially available. Further, the skilled person is aware of cell culture media and conditions that are suitable for culturing and proliferating T cells. When comparing the proliferation of T cells between two cell cultures, e.g., a first culture of T cells comprising an engineered TCR-CD3 complex and a second culture of T cells comprising the non-engineered counterpart of said TCR-CD3 complex, it is preferred that the conditions (cell type, antigen, culture volume, culturing conditions, etc.) in both cell cultures are comparable.

In certain embodiments, a T cells comprising an engineered TCR-CD3 complex of the invention proliferate at a similar rate as T cells comprising the non-engineered counterpart of the engineered TCR-CD3 complex when contacted with a CD3 agonist. T cells comprising an engineered TCR-CD3 complex are defined to proliferate at a similar rate as T cells comprising the non-engineered counterpart of said engineered TCR-CD3 complex, if a culture of T cells comprising the engineered TCR-CD3 complex contains at least 50%, 60%, 70%, 80%, 90% of the T cells contained in a comparable culture of T cells comprising the non-engineered counterpart of the engineered TCR-CD3 complex when both cultures were contacted with a CD3 agonist for at least 1, 2, 3, 4, 5 days. In a preferred embodiment, T cells comprising an engineered TCR-CD3 complex are defined to proliferate at a similar rate as T cells comprising the non-engineered counterpart of said engineered TCR-CD3 complex, if a culture of T cells comprising the engineered TCR-CD3 complex contains at least 50% of the T cells contained in a comparable culture of T cells comprising the non-engineered counterpart of the engineered TCR-CD3 complex when both cultures were contacted with a CD3 agonist for 5 days.

In certain embodiments, T cell comprising an engineered TCR-CD3 complex of the invention proliferate at a lower rate than T cells comprising the non-engineered counterpart of the engineered TCR-CD3 complex when contacted with a cognate peptide-MHC complex. T cells comprising an engineered TCR-CD3 complex are defined to proliferate at a lower rate than T cells comprising the non-engineered counterpart of the engineered TCR-CD3 complex, if the number of T cells in a culture of T cells comprising the engineered TCR-CD3 is reduced by at least 50%, 60%, 70%, 80%, 90% compared to the number of T cells in a comparable culture of T cells comprising the non-engineered counterpart of said engineered TCR-CD3 complex when both cultured were contacted with a cognate peptide-MHC complex for at least 1, 2, 3, 4, 5 days. In a preferred embodiment, T cells comprising an engineered TCR-CD3 complex are defined to proliferate at a lower rate than T cells comprising the non-engineered counterpart of the engineered TCR-CD3 complex, if the number of T cells in a culture of T cells comprising the engineered TCR-CD3 is reduced by at least 50% compared to the number of T cells in a comparable culture of T cells comprising the non-engineered counterpart of said engineered TCR-CD3 complex when both cultured were contacted with a cognate peptide-MHC complex for 5 days.

In particular, the proliferation rate of T cells may be quantified as follows:

T cells may be co-cultured with antigen-presenting cells as described elsewhere herein. Preferably, T cells are co-cultured with antigen-presenting cells at a ratio of 1:10. Over a period of 5 days, the ratio of cells in a culture may be determined by flow cytometry and the proliferation rate may be quantified as the percentage of T cells in the culture.

Similarly, T cells may be contacted with a CD3 agonist as described herein above for a defined period of time and the number of T cells in the culture may be determined by flow cytometry at various time points. To determine the proliferation rate, it is required to compare the number of T cells comprising an engineered TCR-CD3 complex in a first culture with the number of T cells comprising the non-engineered counterpart of the TCR-CD3 complex in a second culture. For that, it is preferred that both cell cultures had a similar cell count at the beginning of the experiment and that the T cells in both cultures have been contacted with a CD3 agonist. In a preferred embodiment, the first and second culture comprise 5×10³ T cells which are contacted with 12 ng/ml blinatumomab for 5 days. If the number of T cells in the culture comprising the T cells comprising the engineered TCR-CD3 complex is at least 50% of the number of T cells in the comprising the T cell comprising the non-engineered counterpart of the TCR-CD3 complex after the 5 day incubation period, a similar proliferation is observed.

In certain embodiments, the CD3 agonist is a bispecific antibody. In such embodiments, the proliferation of T cells may be determined in a co-culture assay. For example, the proliferation of T cells in the presence of the bispecific antibody blinatumomab may be determined by co-culturing T cells with a CD19-expressing target cell, such as a Raji cell. T cells and CD19-expressing cells may be co-cultured as disclosed herein and contacted with blinatumomab. Binding of blinatumomab to the T cell may induce proliferation of the T cell while binding of blinatumomab may have no significant effect on the proliferation of CD19-expressing cells. As such, the ratio between T cells and CD19-expressing cells will increase after contacting the co-cultured cells with blinatumomab. The proliferation rate may be quantified as the percentage of T cells in the culture. As such, a T cell comprising an engineered TCR-CD3 complex may be determined to have a similar proliferation rate as a T cell comprising the non-engineered counterpart of the engineered TCR-CD3 complex, if the percentage of T cells in a co-culture comprising T cells comprising an engineered TCR-CD3 complex and CD19-expressing cells is at least 50% of the percentage of T cells in a co-culture comprising T cells comprising the non-engineered counterpart of the engineered TCR-CD3 complex and CD19-expressing cells, after both co-cultures have been contacted with 12 ng/mL blinatumomab for 5 days.

### The T Cell

The engineered TCR-CD3 complex of the invention is preferably comprised in a T cell. The terms “T cell” and “T lymphocyte” are used interchangeably herein and include T helper cells (CD4+ T cells) and cytotoxic T cells (CTLs, CD8+ T cells) which comprise cytolytic T cells.

T cells belong to a group of white blood cells known as lymphocytes and play a central role in cell-mediated immunity. They can be distinguished from other lymphocyte types, such as B cells and natural killer cells by the presence of a special receptor on their cell surface called T cell receptors (TCR). The thymus is the principal organ responsible for the T cell's maturation of T cells. Several different subsets of T cells have been discovered, each with a distinct function.

T helper cells assist other white blood cells in immunologic processes, including maturation of B cells into plasma cells and activation of cytotoxic T cells and macrophages, among other functions. These cells are also known as CD4+ T cells because they express the CD4 protein on their surface. Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8+ T cells since they express the CD8 glycoprotein at their surface. These cells recognize their targets by binding to antigen associated with MHC class I, which is present on the surface of nearly every cell of the body.

A majority of T cells have a T cell receptor (TCR) existing as a complex of several proteins. The actual T cell receptor is composed of two separate peptide chains, which are produced from the independent T cell receptor alpha and beta (TCRa and TCRβ) genes and are called α- and β-TCR chains. γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the αβ T cells.

The structure of the T cell receptor is very similar to immunoglobulin Fab fragments, which are regions defined as the combined light and heavy chain of an antibody arm. Each chain of the TCR is a member of the immunoglobulin superfamily and possesses one N-terminal variable (V) domain, one constant (C) domain, a transmembrane/cell membrane-spanning region, and a short cytoplasmic tail at the C-terminal end.

The variable region of both the TCR α-chain and β-chain have three hypervariable or complementarity determining regions (CDRs), whereas the variable region of the β-chain has an additional area of hypervariability (HV4) that does not normally contact antigen and therefore is not considered a CDR. CDR3 is the main CDR responsible for recognizing processed antigen, although CDR1 of the α-chain has also been shown to interact with the N-terminal part of the antigenic peptide, whereas CDR1 of the β-chain interacts with the C-terminal part of the peptide. CDR2 is thought to recognize the MHC. CDR4 of the β-chain is not thought to participate in antigen recognition, but has been shown to interact with superantigens.

The constant domain of the TCR domain consists of short connecting sequences in which a cysteine residue forms disulfide bonds, which forms a link between the two chains.

All T cells originate from hematopoietic stem cells in the bone marrow. Hematopoietic progenitors derived from hematopoietic stem cells populate the thymus and expand by cell division to generate a large population of immature thymocytes. The earliest thymocytes express neither CD4 nor CD8, and are therefore classed as double-negative (CD4−CD8−) cells. As they progress through their development, they become double-positive thymocytes (CD4+CD8+), and finally mature to single-positive (CD4+CD8− or CD4−CD8+) thymocytes that are then released from the thymus to peripheral tissues.

The first signal in activation of T cells is provided by binding of the T cell receptor to a short peptide presented by the major histocompatibility complex (MHC) on another cell. This ensures that only a T cell with a TCR specific to that peptide is activated. The partner cell is usually a professional antigen presenting cell (APC), usually a dendritic cell in the case of naive responses, although B cells and macrophages can be important APCs. The peptides presented to CD8+ T cells by MHC class I molecules are 8-10 amino acids in length; the peptides presented to CD4+ T cells by MHC class II molecules are longer, as the ends of the binding cleft of the MHC class II molecule are open.

T cells may generally be prepared in vitro or ex vivo, using standard procedures. For example, T cells may be present within (or isolated from) bone marrow, peripheral blood or a fraction of bone marrow or peripheral blood of a mammal, such as a patient, using a commercially available cell separation system. Alternatively, T cells may be derived from related or unrelated humans, non-human animals, cell lines or cultures. A “sample comprising T cells” may, for example, be peripheral blood mononuclear cells (PBMC).

Alternatively, and as specified elsewhere herein, T cell may be derived from induced pluripotent stem cells (iPSCs) that have been obtained from somatic cells.

T cells may be stimulated with antigen, peptide, nucleic acid and/or antigen presenting cells (APCs) that express an antigen. Such stimulation is performed under conditions and for a time sufficient to permit the generation of T cells that are specific for an antigen, a peptide and/or cells presenting an antigen or a peptide. Alternatively, T cells may be activated ex vivo in an antigen-independent manner by using a CD3 agonist, such as microbeads coupled to anti-CD3 and anti-CD28 antibodies.

Specific activation of CD4+ or CD8+ T cells may be detected in a variety of ways. Methods for detecting specific T cell activation include detecting the proliferation of T cells, the production of cytokines (e.g., lymphokines), or the generation of cytolytic activity. For CD4+ T cells, a preferred method for detecting specific T cell activation is the detection of the proliferation of T cells. For CD8+ T cells, a preferred method for detecting specific T cell activation is the detection of the generation of cytolytic activity.

In certain embodiments, the T cell of the invention is a CD8+ T cell. In certain embodiments, the T cell of the invention is a CD4+ T cell. In certain embodiments, the T cell of the invention is a human CD4+ or CD8+ T cell. The skilled person is aware of methods to identify and/or isolate CD4+ T cells and CD8+ T cells. Methods commonly used in the art to identify and/or isolate CD4+ or CD8+ T cells include flow cytometry.

### The CD3 Agonist

The term “CD3 agonist” as used herein, in its broadest meaning, refers to a molecule that can directly or indirectly stimulate CD3 in order to generate a CD3 activation signal. That is, the CD3 agonist is a molecule that can activate T cells in a CD3-dependent manner.

In certain embodiments, the CD3 agonist is a molecule that directly interacts with CD3, i.e., a CD3-binding agent. That is, the CD3 agonist according to the invention may be, without limitation, an antibody, an antibody fragment, an antibody mimic, an antibody fusion protein, an affimer, an aptamer, an alphabody, an anticalin, an avimer, a DARPin, a fynomer, a Kunitz domain peptide, or a monobody.

In a particular embodiment, the invention relates to the T cell according to the invention, wherein the CD3 agonist is an anti-CD3 antibody or a fragment thereof. In another preferred embodiment, the CD3 agonist is a fusion protein comprising an anti-CD3 antibody or a fragment thereof.

The term “antibody” refers to a polypeptide or group of polypeptides that comprise at least one antigen-binding site. An “antigen binding site” is formed from the folding of the variable domains of an antibody molecule(s) to form three-dimensional binding sites with an internal surface shape and charge distribution complementary to the features of an epitope of an antigen, which allows specific binding to form an antibody/antigen complex. An antigen-binding site may be formed from a heavy- and/or light-chain domain (VH and VL, respectively), which form hypervariable loops that contribute to antigen binding. The anti-CD3 agonist antibody may be, without limitation, a monoclonal antibody, a polyclonal antibody, a human antibody, a murine antibody, a humanized antibody, a recombinant antibody, a chimeric antibody, a fusion antibody or a multispecific antibody.

The term “antibody fragment” as used herein refers to an incomplete or isolated portion of the full sequence of the antibody which retains the antigen binding function of the parent antibody. Examples of antibody fragments include Fab, Fab′, F(ab′)2, and Fv fragments; diabodies; linear antibodies; single-chain antibody molecules; and multispecific antibodies formed from antibody fragments.

Agonistic anti-CD3 antibodies are known in the art and are widely used for the activation of T cells in a CD3-dependent manner. Several agonistic anti-CD3 antibodies for activating T cells ex vivo are known in the art and are commercially available. These antibodies may be naked antibodies or may be immobilized on a solid surface, such as a microtiter plate or a particle such as a microbead. The agonistic anti-CD3 antibody may also be an antibody that is suitable for the use in therapy, such as a monoclonal and/or human/humanized antibody.

In a particularly preferred embodiment, the CD3 agonist is a multispecific antibody. The term “multispecific antibody” as used herein, refers to an antibody that binds to two or more different epitopes on at least two or more different targets (e.g., CD3 and CD19). The term “multispecific antibody” includes bispecific, trispecific, tetraspecific, pentaspecific and hexaspecific antibodies. The term “bispecific antibody” as used herein, refers to an antibody that binds to two different epitopes on at least two different targets (e.g., CD3 and CD19).

In a particular embodiment, the invention relates to the T cell according to the invention, wherein the anti-CD3 antibody is a bispecific antibody.

CD3-bispecific antibodies (CD3-BsAbs) are an emerging treatment modality in the field of cancer immunotherapy. BsAbs can recognize distinct antigens with each of their antigen-binding domains, in contrast to conventional Abs that recognize the same antigen with both Fab arms. CD3-BsAbs act by simultaneous binding to a tumor-associated antigen (TAA) expressed on tumor cells and to CD3 on a T cell. Crosslinking of these two cell types by CD3-BsAbs allows the formation of an immunological synapse. This synapse results in T-cell activation and thereby the secretion of inflammatory cytokines and cytolytic molecules that are able to kill the tumor cells in the process. The strength of CD3-BsAbs lies in the fact that any T cell could serve as an effector cell, regardless of TCR specificity, as for these BsAbs, TCR signaling does not require engagement of the antigen-binding domain of the TCR, but is initiated via CD3. Therefore, CD3-BsAbs can employ all available T cells and are not limited to tumor-specific T cells, contrary to the key requirement for effective immune checkpoint therapy.

CD3-BsAbs received a lot of attention due to their success in hematological cancers. Blinatumomab (a CD3/CD19 BsAb without an Fc tail) was FDA approved in 2014 and is now successfully used in the clinic to treat patients suffering from relapsed or refractory B-cell precursor acute lymphoblastic leukemia (ALL). Thus, in a particularly preferred embodiments, the CD3 agonist according to the invention is a bispecific anti-CD3/anti-CD19 antibody. In certain embodiments, the bispecific anti-CD3/anti-CD19 antibody may be blinatumomab.

Apart from blinatumomab, many other CD3-BsAbs are currently in clinical trials targeting well-established B-cell markers, like CD19, CD20, CD38 and B-cell maturation antigen (BCMA) and myeloid markers, like CD33 and CD123. Thus, in certain embodiments, the CD3 agonist may be a bispecific antibody that specifically binds to CD3 and one tumor antigen selected from the group consisting of: CD19, CD20, CD38, BCMA, CD33 and CD123.

In certain embodiments, the CD3 agonist may bind to CD3 and additionally target a tumor antigen that is characteristic for solid tumors. That is, the CD3 agonist may be a bispecific antibody that specifically binds to CD3 and one tumor antigen selected from the group consisting of: carcinoembryonic antigen (CEA), epidermal growth factor receptor (EGFR), EpCAM, HER2, prostate-specific membrane antigen (PSMA), B7-H3, B7-H4, CD133, CD155, claudin 6, claudin 18.2, cellular mesenchymal to epithelial transcription factor C (C-MET), ephrin receptor A10 (EphA10), folate receptor 1 (FOLR1), HLA-A*24: surviving 2B80-88, HLA-A*02:01:gp100 integrin β4 (ITGB4), HLA-A*02: MAGE-A4, P-cadherin, prolactin receptor (PRLR), receptor tyrosine kinase-like orphan receptor 1 (ROR1), TNF-related apoptosis-inducing ligand receptor (TRAIL-R2), GD2, gpA33, GPC3, 5T4, MUC1, MUC16, MUC17, NY-ESO1, PRAME, PSCA, SSTR2, STEAP1, DLL3, GUCY2C, MSLN, transferrin receptor (TfR) and tumor-associated calcium signal transducer 2 (Trop-2).

In certain embodiments, the CD3 agonist is a bispecific antibody that specifically binds to CD3 and a tumor antigen. In certain embodiments, the CD3 agonist is a bispecific antibody that specifically binds to CD3 and a viral antigen.

The bispecific CD3 agonist of the invention does not necessarily have to be an antibody, but may also be a fusion protein comprising a CD3-binding domain and a binding domain for one additional antigen, preferably a tumor or viral antigen. A non-limiting example of such bispecific fusion proteins are ImmTACs (Immune mobilising monoclonal T-cell receptors Against Cancer). ImmTACs are a class of bispecific biological drugs being investigated for the treatment of cancer and viral infections which combine engineered cancer-recognizing TCRs with immune activating complexes. ImmTACs target cancerous or virally infected cells through binding human leukocyte antigen (HLA) presented peptide antigens and redirect the host's cytotoxic T cells to recognize and kill them. ImmTACs are fusion proteins that combine an engineered T Cell Receptor (TCR) based targeting system with a single chain antibody fragment (scFv) effector function. The TCR portion of the ImmTAC preferably binds to a tumor antigen or a viral antigen. More preferably, the TCR portion of the ImmTAC binds to one of the tumor antigens disclosed herein. In a particular embodiment, the ImmTAC specifically binds to the melanoma associated antigen gp100.

Thus, in a particular embodiment, the invention relates to the T cell according to the invention, wherein the anti-CD3 antibody fragment is comprised in a fusion protein. In a particular embodiment, the invention relates to the T cell according to the invention, wherein the fusion protein further comprises a fragment of a T cell receptor.

In certain embodiments, the CD3 agonist may indirectly stimulate CD3 in order to generate a CD3 activation signal. That is, the CD3 agonist may not directly interact with CD3 but may activate CD3 through an intermediate player. For example, it has been demonstrated by Bockenstedt et al. (J Immunol, 1988, 141 (6), 1904-1911) that antibodies binding specifically to CD2 can activate T cells in a CD3-dependent manner. Thus, in certain embodiments, the CD3 agonist may be a CD2-binding agent, in particular an anti-CD2 antibody. In certain embodiments, the CD3 agonist may be a bispecific antibody that specifically binds to CD2 and any one of the antigens disclosed above. In certain embodiments, the CD3 agonist may be a bispecific antibody that specifically binds to CD2 and a tumor antigen.

### The Engineered TCR-CD3 Complex

The present invention is based on the discovery of an engineered TCR-CD3 complex. It is preferred herein, that the engineered TCR-CD3 complex comprises at least one mutation in a TCR chain. In certain embodiments, the engineered TCR-CD3 complex only comprises mutations in the TCR chains, whereas the CD3 portion of the engineered TCR-CDR complex remains unmodified.

In certain embodiments, the one or more mutation(s) in the engineered TCR-CD3 complex are located in the TCR alpha and/or beta chain. Thus, in a particular embodiment, the invention relates to the T cell according to the invention, wherein the engineered TCR-CD3 complex comprises at least one mutation in a TCR alpha and/or beta chain.

Preferably, the one or more mutation is located in a conserved sequence motif in a TCR alpha and/or beta chain. The highest degree of sequence variability in TCRs can be found in the complementarity determining regions (CDRs). Thus, in a particular embodiment, the invention relates to the T cell according to the invention, wherein the at least one mutation in the TCR alpha and/or beta chain is located outside of a complementary determining region (CDR).

It has been shown by the inventors that introducing mutations at the interface between the TCR alpha and beta chain results in efficient decoupling of TCR-antigen binding from the generation of a CD3 activation signal. Without wishing to be being bound by any theory, introducing mutations at the interface between the TCR alpha and beta chain may compromise the integrity of the TCR-peptide-MHC interface, resulting in reduced structural movements and consequently reduced signal transduction to CD3 molecules. Thus, in a particular embodiment, the invention relates to the T cell according to the invention, wherein the at least one mutation in the TCR alpha and/or beta chain is located at the interface between the TCR alpha and beta chain. In a particular embodiment, the invention relates to the T cell according to the invention, wherein the at least one mutation in the TCR alpha and/or beta chain is located at the TCR-peptide-MHC interface. The skilled person is aware of methods and software tools to identify residues that are located at the interface of the TCR alpha and beta subunit or the interface of the TCR and a peptide-MHC complex.

Again, without wishing to be being bound by any theory, introducing mutations at the interface of the TCR alpha and beta chain may weaken the TCR-alpha-beta association and thus inhibit signal transduction to the CD3 subunits. Thus, in a particular embodiment, the invention relates to the T cell according to the invention, wherein the at least one mutation in the TCR alpha and/or beta chain results in reduced TCR alpha and beta association. Whether a mutation results in reduced TCR alpha and beta chain association may be determined by methods known in the art, such as immunoprecipitation. For example, the association between TCR alpha and beta chains may be analyzed as disclosed by Li et al., Immunology, 1996, 88, 524-530).

The inventors identified several sequence motifs that are highly conserved among αβ TCRs. Thus, in a particular embodiment, the invention relates to the T cell according to the invention, wherein the at least one mutation in the TCR alpha and/or beta chain has been introduced in the motif WYRQ, FG₁xG₂T, VxP, PDP, FTDFDS and/or FETDxNLN, wherein x is an undefined amino acid. However, it is to be understood that the engineered TCR-CD3 complex may comprise any mutation in a TCR chain that results in a TCR-CD3 complex having the functional features as defined herein.

### The Sequence Motif FGxGT

The inventors identified that mutations in the sequence motif FGxGT (SEQ ID NO:88) result in the decoupling of TCR-antigen binding from the generation of a CD3 activation signal. Thus, in a particular embodiment, the invention relates to the T cell according to the invention, wherein the at least one mutation in the TCR alpha and/or beta chain has been introduced into the motif FG₁xG₂T, wherein x is an undefined amino acid

The motif FGxGT is highly conserved in the TCR alpha and beta chain. It is encoded by the J genes (TRAJ and TRBJ) and is thus comprised in the variable domain of the TCR alpha and beta chain. As it is shown in FIG. 2B, positions 1, 2 4 and 5 of the motif FGxGT are highly conserved among human TRAJ genes, wherein positions 3 (x) may vary. Despite the high degree of sequence conservation, it is to be understood that in certain naturally occurring J genes, one or more amino acid residues in positions 1, 2, 4 or 5 of the motif FGxGT may deviate. For example, the beta chain of the human anti-NY-ESO-1 T cell receptor 1G4 comprises the sequence FGEGS (SEQ ID NO:91). Thus, it is to be understood that “the motif FGxGT” also encompasses variants that may deviate from the consensus sequence, such as the sequence FGEGS of the human anti-NY-ESO-1 T cell receptor 1G4. Furthermore, it is to be understood that depending on the VJ-gene usage and/or the length of the CDRs, the IMGT numbering of the motif FGxGT may slightly deviate. However, the skilled person is capable of identifying the motif FGxGT in any given TCR alpha and/or beta chain based on the information provided herein.

To facilitate the identification of the sequence motif FGxGT in TCRs, a numbering scheme may be used. To compare the variable domains of different TCR, the IMGT numbering scheme is commonly used (Lefranc, Immunology Today, 1997, 18, 509; Lefranq, The Immunologist, 1999, 7, 132-136; Lefranq et al., Dev. Comp. Immunol., 2003, 27, 55-77; http://www.imgt.org/IMGTScientificChart/Numbering/IMGTIGVLsuperfamily.html). In the IMGT numbering scheme, conserved amino acid residues in the variable domain always have the same position. However, other numbering schemes for TCR variable domains are known in the art, such as Kabat, Clothia, Martin or AHo (http://opig.stats.ox.ac.uk/webapps/newsabdab/sabpred/anarci/).

The sequence motif FGxGT is located at position 118-122 (IMGT) of the TCR alpha and beta chain. Thus, in certain embodiments, the engineered TCR-CD3 complex according to the invention may comprise one or more mutation in any one of positions 118, 119, 120, 121 and/or 122 of the TCR alpha and/or beta chain (according to IMGT numbering). In certain embodiments, the engineered TCR-CD3 complex according to the invention comprises a mutation at position 118 (IMGT) of the TCR alpha and/or beta chain, in particular at position F118 (IMGT). In certain embodiments, the engineered TCR-CD3 complex according to the invention comprises a mutation at position 119 (IMGT) of the TCR alpha and/or beta chain, in particular at position G119 (IMGT). In certain embodiments, the engineered TCR-CD3 complex according to the invention comprises a mutation at position 121 (IMGT) of the TCR alpha and/or beta chain, in particular at position G121 (IMGT). In certain embodiments, the engineered TCR-CD3 complex according to the invention comprises a mutation at position 122 (IMGT) of the TCR alpha and/or beta chain, in particular at position T122 or S122 (IMGT).

In certain embodiments, the engineered TCR-CD3 complex according to the invention comprises mutations at position 119 (IMGT) and 121 (IMGT) of the TCR alpha and/or beta chain, in particular at position G119 (IMGT) and G121 (IMGT). Thus, in a particular embodiment, the invention relates to the T cell according to the invention, wherein the residue G₁ and/or G₂ of the motif FG₁xG₂T in the TCR alpha and/or beta chain has been replaced with another amino acid.

In certain embodiments, the engineered TCR-CD3 complex according to the invention comprises mutations at position 118 (IMGT) and 119 (IMGT) of the TCR alpha and/or beta chain, in particular at position F118 (IMGT) and G119 (IMGT). However, it is to be understood that any combination of two or more mutations in the amino acid residues 118, 119, 121 and 122 (IMGT) in a TCR alpha and/or beta chain may result in an engineered TCR-CD3 complex according to the invention.

In certain embodiments, the invention relates to an engineered TCR-CD3 complex comprising the MART-1 specific TCR DMF5 (SEQ ID NO:1 and 2).

The alpha chain variable region of DMF5 (SEQ ID NO:1) comprises the sequence motif FGQGT at IMGT positions 118-122 (corresponding to residues 99-103 of SEQ ID NO:1) That is, residue F118 (IMGT) corresponds to residue F99 of SEQ ID NO:1; residue G119 (IMGT) corresponds to residue G100 of SEQ ID NO:1, residue Q120 (IMGT) corresponds to residue Q101 of SEQ ID NO: 1, residue G121 (IMGT) corresponds to residue G102 of SEQ ID NO:1 and residue T122 (IMGT) corresponds to residue T103 of SEQ ID NO:1.

The beta chain variable region of DMF5 (SEQ ID NO:2) comprises the sequence motif FGQGT at IMGT positions 118-122 (corresponding to residues 103-107 of SEQ ID NO:2) That is, residue F118 (IMGT) corresponds to residue F103 of SEQ ID NO:2; residue G119 (IMGT) corresponds to residue G104 of SEQ ID NO:2, residue Q120 (IMGT) corresponds to residue Q105 of SEQ ID NO: 2, residue G121 (IMGT) corresponds to residue G106 of SEQ ID NO:2 and residue T122 (IMGT) corresponds to residue T107 of SEQ ID NO:2.

In certain embodiments, the engineered DMF5 variant comprises the mutations G119E and G121W (IMGT), G119W and G121S (IMGT), G119H and G121A (IMGT), G119Y and G121L (IMGT) or G119H and G121V (IMGT) in the TCR alpha and/or beta chain of DMF5. In a preferred embodiment, the engineered DMF5 variant comprises the mutations G119E and G121W (IMGT) in the TCR alpha chain of DMF5. In certain embodiments, the invention relates to an engineered TCR-CD3 complex comprising the MART-1 specific TCR DMF5 (SEQ ID NO:1 and 2), wherein the sequence motif FGQGT (residues 118-122 IMGT) in the TCR alpha and/or beta chain is replaced with the sequence motif FEQWT (SEQ ID NO:9), FWQST (SEQ ID NO:10), FHQAT (SEQ ID NO:11), FYQLT (SEQ ID NO:12) or FHQVT (SEQ ID NO:13).

In certain embodiments, the invention relates to an engineered TCR-CD3 complex comprising the NY-ESO-1 specific TCR IG4 (SEQ ID NO:3 and 4).

The alpha chain variable region of IG4 (SEQ ID NO:3) comprises the sequence motif FGRGT (SEQ ID NO:92) at IMGT positions 118-122 (corresponding to residues 103-107 of SEQ ID NO:3) That is, residue F118 (IMGT) corresponds to residue F103 of SEQ ID NO:3; residue G119 (IMGT) corresponds to residue G104 of SEQ ID NO:3, residue R120 (IMGT) corresponds to residue R105 of SEQ ID NO:3, residue G121 (IMGT) corresponds to residue G106 of SEQ ID NO:3 and residue T122 (IMGT) corresponds to residue T107 of SEQ ID NO:3.

The beta chain variable region of IG4 (SEQ ID NO:4) comprises the sequence motif FGEGS at IMGT positions 118-122 (corresponding to residues 102-106 of SEQ ID NO:4) That is, residue F118 (IMGT) corresponds to residue F102 of SEQ ID NO:4; residue G119 (IMGT) corresponds to residue G103 of SEQ ID NO:4, residue E120 (IMGT) corresponds to residue E104 of SEQ ID NO: 4, residue G121 (IMGT) corresponds to residue G105 of SEQ ID NO:4 and residue S122 (IMGT) corresponds to residue S106 of SEQ ID NO:4.

In certain embodiments, the engineered IG4 variant comprises the mutations G119S and G121W (IMGT), G119V and G121L (IMGT), G119L and G121G (IMGT), G119R and G121A (IMGT) or G119K and G121E (IMGT) in the TCR alpha and/or beta chain of IG4. In certain embodiments, the invention relates to an engineered TCR-CD3 complex comprising the NY-ESO-1 specific TCR IG4 (SEQ ID NO:3 and 4), wherein the sequence motif FGRGT (residues 118-122 IMGT) in the TCR alpha chain and/or FGEGS (residues 118-122 IMGT) in the TCR beta chain is replaced with the sequence motif FSQVT (SEQ ID NO:14), FVOLT (SEQ ID NO:15), FLQGT (SEQ ID NO: 16), FROAT (SEQ ID NO:17) or FKQET (SEQ ID NO:18).

In certain embodiments, the invention relates to an engineered TCR-CD3 complex comprising the MAGE-A3 specific TCR a3a (SEQ ID NO:5 and 6).

The alpha chain variable region of a3a (SEQ ID NO:5) comprises the sequence motif FGKGT at IMGT positions 118-122 (corresponding to residues 104-108 of SEQ ID NO:3) That is, residue F118 (IMGT) corresponds to residue F104 of SEQ ID NO:5; residue G119 (IMGT) corresponds to residue G105 of SEQ ID NO:5, residue K120 (IMGT) corresponds to residue K106 of SEQ ID NO: 5, residue G121 (IMGT) corresponds to residue G107 of SEQ ID NO:5 and residue T122 (IMGT) corresponds to residue T108 of SEQ ID NO:5.

The beta chain variable region of IG4 (SEQ ID NO:6) comprises the sequence motif FGPGT at IMGT positions 118-122 (corresponding to residues 102-106 of SEQ ID NO:6) That is, residue F118 (IMGT) corresponds to residue F102 of SEQ ID NO:6; residue G119 (IMGT) corresponds to residue G103 of SEQ ID NO:6, residue P120 (IMGT) corresponds to residue P104 of SEQ ID NO: 6, residue G121 (IMGT) corresponds to residue G105 of SEQ ID NO:6 and residue T122 (IMGT) corresponds to residue T106 of SEQ ID NO:6.

In certain embodiments, the engineered a3a variant comprises the mutations G119W and G121S (IMGT), G119V and G121L (IMGT) or G119I and G121S (IMGT) in the TCR alpha and/or beta chain of a3a. In certain embodiments, the invention relates to an engineered TCR-CD3 complex comprising the MAGE-A3 specific TCR a3a (SEQ ID NO:5 and 6), wherein the sequence motif FGKGT (residues 118-122 IMGT) in the TCR alpha chain and/or FGPGT (residues 118-122 IMGT) in the TCR beta chain is replaced with the sequence motif FWQST (SEQ ID NO:19), FVOLT (SEQ ID NO:20) or FIQST (SEQ ID NO:21).

In certain embodiments, the invention relates to an engineered TCR-CD3 complex comprising the HCV₁₄₀₆₋₁₄₁₅ specific TCR 1406 (SEQ ID NO:7 and 8).

The alpha chain variable region of 1406 (SEQ ID NO:7) comprises the sequence motif FGKGT at IMGT positions 118-122 (corresponding to residues 106-110 of SEQ ID NO:7) That is residue F118 (IMGT) corresponds to residue F106 of SEQ ID NO:7; residue G119 (IMGT) corresponds to residue G107 of SEQ ID NO:7, residue K120 (IMGT) corresponds to residue K108 of SEQ ID NO: 7, residue G121 (IMGT) corresponds to residue G109 of SEQ ID NO:7 and residue T122 (IMGT) corresponds to residue T110 of SEQ ID NO:7.

The beta chain variable region of 1406 (SEQ ID NO:8) comprises the sequence motif FGPGT at IMGT positions 118-122 (corresponding to residues 104-108 of SEQ ID NO:8) That is residue F118 (IMGT) corresponds to residue F104 of SEQ ID NO:8; residue G119 (IMGT) corresponds to residue G105 of SEQ ID NO:8, residue P120 (IMGT) corresponds to residue P106 of SEQ ID NO: 8, residue G121 (IMGT) corresponds to residue G107 of SEQ ID NO:8 and residue T122 (IMGT) corresponds to residue T108 of SEQ ID NO:8.

In certain embodiments, the engineered 1406 variant comprises the mutations G119S and G121V (IMGT) or G119R and G121A (IMGT) in the TCR alpha and/or beta chain of 1406. In certain embodiments, the invention relates to an engineered TCR-CD3 complex comprising the HCV₁₄₀₆₋₁₄₁₅ specific TCR 1406 (SEQ ID NO:7 and 8), wherein the sequence motif FGKGT (residues 118-122 IMGT) in the TCR alpha chain and/or FGPGT (residues 118-122 IMGT) in the TCR beta chain is replaced with the sequence motif FSQVT (SEQ ID NO:22), FRQAT (SEQ ID NO: 23) or FIQST (SEQ ID NO:24).

Preferably, the above-mentioned mutations are introduced into the TCR beta chain.

### The Sequence Motif WYRQ

The inventors further identified the sequence motif WYRQ (SEQ ID NO:87) to be highly conserved among TCRs and thus as a preferred target to achieve decoupling of TCR-antigen binding from the generation of a CD3 activation signal. In a particular embodiment, the invention relates to the T cell according to the invention, wherein the at least one mutation in the TCR alpha and/or beta chain has been introduced into the motif WYRQ.

The motif WYRQ is highly conserved in the TCR alpha and beta chain of TCRs. It is encoded by the V genes (TRAV and TRBV) and is thus comprised in the variable domain of the TCR alpha and beta chain. Despite the high degree of sequence conservation, it is to be understood that in certain naturally occurring V genes, one or more amino acid residues in the WYRQ motif may deviate. For example, the alpha chain of IG4 and a3a comprises the sequence WFRQ (SEQ ID NO: 94). Further, the beta chain of a3a comprises the sequence WYQQ (SEQ ID NO:95). Thus, it is to be understood that “the motif WYRQ” also encompasses variants that may deviate from the consensus sequence, such as the sequence WFRQ in the alpha chain of the human anti-NY-ESO-1 T cell receptor 1G4. Furthermore, it is to be understood that depending on the VJ-gene usage and/or the length of the CDRs, the IMGT numbering of the motif WYRQ may slightly deviate. However, the skilled person is capable of identifying the motif WYRQ in any given TCR alpha and/or beta chain based on the information provided herein.

According to the IMGT numbering scheme, the sequence motif WYRQ (or variations thereof) can be found at position 41-44 (IMGT) of the TCR alpha and beta chain. Thus, in certain embodiments, the engineered TCR-CD3 complex according to the invention may comprise one or more mutations in any one of positions 41, 42, 43 and/or 44 of the TCR alpha and/or beta chain (according to IMGT numbering). In certain embodiments, the engineered TCR-CD3 complex according to the invention comprises a mutation at position 41 (IMGT) of the TCR alpha and/or beta chain, in particular at position W41 (IMGT). In certain embodiments, the engineered TCR-CD3 complex according to the invention comprises a mutation at position 42 (IMGT) of the TCR alpha and/or beta chain, in particular at position Y42 or F42 (IMGT). In certain embodiments, the engineered TCR-CD3 complex according to the invention comprises a mutation at position 43 (IMGT) of the TCR alpha and/or beta chain, in particular at position R43 or Q43 (IMGT). In certain embodiments, the engineered TCR-CD3 complex according to the invention comprises a mutation at position 44 (IMGT) of the TCR alpha and/or beta chain, in particular at position Q44 (IMGT). In certain embodiments, the engineered TCR-CD3 complex according to the invention comprises mutations at position 42 (IMGT) and 44 (IMGT) of the TCR alpha and/or beta chain, in particular at position Y42 (IMGT) and Q44 (IMGT). However, it is to be understood that any combination of two or more mutations in the amino acid residues 41, 42, 43 and 44 (IMGT) in a TCR alpha and/or beta chain may result in an engineered TCR-CD3 complex according to the invention.

In certain embodiments, the invention relates to an engineered TCR-CD3 complex comprising the MART-1 specific TCR DMF5 (SEQ ID NO:1 and 2), wherein the MART-1 specific TCR DMF5 comprises at least one mutation in the sequence motif WYRQ of the TCR alpha (residues 34-37 of SEQ ID NO:1) and/or beta chain (residues 34-37 of SEQ ID NO:2). In certain embodiments, the invention relates to an engineered TCR-CD3 complex comprising the NY-ESO-1 specific TCR IG4 (SEQ ID NO:3 and 4), wherein the NY-ESO-1 specific TCR IG4 comprises at least one mutation in the sequence motif WFRQ of the TCR alpha chain (residues 34-37 of SEQ ID NO:3) and/or in the sequence motif WYRQ of the TCR beta chain (residues 32-35 of SEQ ID NO:4). In certain embodiments, the invention relates to an engineered TCR-CD3 complex comprising the MAGE-A3 specific TCR a3a (SEQ ID NO:5 and 6), wherein the MAGE-A3 specific TCR a3a comprises at least one mutation in the sequence motif WFRQ of the TCR alpha chain (residues 35-38 of SEQ ID NO:5) and/or in the sequence motif WYQQ of the TCR beta chain (residues 33-36 of SEQ ID NO:6). In certain embodiments, the invention relates to an engineered TCR-CD3 complex comprising the HCV₁₄₀₆₋₁₄₁₅ specific TCR 1406 (SEQ ID NO:7 and 8), wherein the HCV₁₄₀₆₋₁₄₁₅ specific TCR 1406 comprises at least one mutation in the sequence motif WYKQ (SEQ ID NO:96) of the TCR alpha chain (residues 40-43 of SEQ ID NO:7) and/or in the sequence motif WYQQ (SEQ ID NO:97) of the TCR beta chain (residues 36-39 of SEQ ID NO:8).

### The Sequence Motif VxP

The inventors further identified the sequence motif VxP to be highly conserved among TCRs and thus as a preferred target to achieve decoupling of TCR-antigen binding from the generation of a CD3 activation signal. Thus, in a particular embodiment, the invention relates to the T cell according to the invention, wherein the at least one mutation in the TCR alpha and/or beta chain has been introduced into the motif VxP, wherein x is an undefined amino acid.

The motif VxP is highly conserved in the TCR alpha and beta chain of TCRs. It is encoded by the J genes (TRAJ and TRBJ) and is thus comprised in the variable domain of the TCR alpha and beta chain. Despite the high degree of sequence conservation, it is to be understood that in certain naturally occurring J genes, one or more amino acid residues in the VxP motif may deviate, in particular in the beta chain. For example, the beta chain of DMF5 comprises the sequence VVE, the beta chain of IGT comprises the sequence VLE and the beta chain of A3A comprises the sequence VTE. Thus, it is to be understood that “the motif VxP” also encompasses variants that may deviate from the consensus sequence, such as the sequence VVE in the beta chain of the T cell receptor DMF5. Furthermore, it is to be understood that depending on the VJ-gene usage and/or the length of the CDRs, the IMGT numbering of the motif VxP may slightly deviate. However, the skilled person is capable of identifying the motif VxP in any given TCR alpha and/or beta chain based on the information provided herein.

According to the IMGT numbering scheme, the sequence motif VxP (or variations thereof) can be found at position 126-128 (IMGT) of the TCR alpha and beta chain. Thus, in certain embodiments, the engineered TCR-CD3 complex according to the invention may comprise one or more mutations in any one of positions 126, 127 and/or 128 of the TCR alpha and/or beta chain (according to IMGT numbering). In certain embodiments, the engineered TCR-CD3 complex according to the invention comprises a mutation at position 126 (IMGT) of the TCR alpha and/or beta chain, in particular at position V126 (IMGT). In certain embodiments, the engineered TCR-CD3 complex according to the invention comprises a mutation at position 128 (IMGT) of the TCR alpha and/or beta chain, in particular at position P128 or E128 (IMGT). In certain embodiments, the engineered TCR-CD3 complex according to the invention comprises mutations at position 126 (IMGT) and 128 (IMGT) of the TCR alpha and/or beta chain, in particular at position V126 (IMGT) and P128 (IMGT). In certain embodiments, the engineered TCR-CD3 complex according to the invention comprises mutations at position 126 (IMGT) and 128 (IMGT) of the TCR alpha and/or beta chain, in particular at position V126 (IMGT) and E128 (IMGT). However, it is to be understood that any combination of two or more mutations in the amino acid residues 126, 127 and 128 (IMGT) in a TCR alpha and/or beta chain may result in an engineered TCR-CD3 complex according to the invention.

In certain embodiments, the invention relates to an engineered TCR-CD3 complex comprising the MART-1 specific TCR DMF5 (SEQ ID NO:1 and 2), wherein the MART-1 specific TCR DMF5 comprises at least one mutation in the sequence motif VKP of the TCR alpha chain (residues 107-109 of SEQ ID NO:1) and/or in the sequence motif VVE of the TCR beta chain (residues 111-113 of SEQ ID NO:2). In certain embodiments, the invention relates to an engineered TCR-CD3 complex comprising the NY-ESO-1 specific TCR IG4 (SEQ ID NO:3 and 4), wherein the NY-ESO-1 specific TCR IG4 comprises at least one mutation in the sequence motif VHP of the TCR alpha chain (residues 111-113 of SEQ ID NO:3) and/or in the sequence motif VLE of the TCR beta chain (residues 110-112 of SEQ ID NO:4). In certain embodiments, the invention relates to an engineered TCR-CD3 complex comprising the MAGE-A3 specific TCR a3a (SEQ ID NO:5 and 6), wherein the MAGE-A3 specific TCR a3a comprises at least one mutation in the sequence motif VIP of the TCR alpha chain (residues 112-114 of SEQ ID NO:5) and/or in the sequence motif VTE of the TCR beta chain (residues 110-112 of SEQ ID NO:6). In certain embodiments, the invention relates to an engineered TCR-CD3 complex comprising the HCV₁₄₀₆₋₁₄₁₅ specific TCR 1406 (SEQ ID NO:7 and 8), wherein the HCV₁₄₀₆₋₁₄₁₅ specific TCR 1406 comprises at least one mutation in the sequence motif ILP of the TCR alpha chain (residues 114-116 of SEQ ID NO:7) and/or in the sequence motif TVT of the TCR beta chain (residues 111-113 of SEQ ID NO:8).

The conserved sequence motifs discussed above (FGxGT, WYRQ and VxP) are located in the variable chain of the TCR. Thus, in a particular embodiment, the invention relates to the engineered TCR-CD3 complex according to the invention, wherein the one or more mutation is located in the variable domain of the TCR alpha and/or beta chain. In a preferred embodiment, the invention relates to the engineered TCR-CD3 complex according to the invention, wherein the one or more mutation is located at position W41, Y/F42, R/Q43, Q44, F118, G119, G121, T/S122, V126 and/or P128 (all IMGT) of the TCR alpha chain and/or at position W41, Y/F42, R/Q43, Q44, F118, G119, G121, T/S122, T125 and/or V/L/T127 (all IMGT) of the TCR beta chain, wherein the positions in the TCR alpha and/or beta chain are according to the IMGT numbering scheme.

Thus, in a particular embodiment, the invention relates to a T cell comprising an engineered TCR-CD3 complex, wherein the engineered TCR-CD3 complex comprises one or more mutations at positions W41, Y/F42, R/Q43, Q44, F118, G119, G121, T/S122, V126 and/or P128 (all IMGT) of the TCR alpha chain and/or at position W41, Y/F42, R/Q43, Q44, F118, G119, G121, T/S122, T125 and/or V/L/T127 (all IMGT) of the TCR beta chain, wherein the positions in the TCR alpha and/or beta chain are according to the IMGT numbering scheme, and wherein the one or more mutations result in decoupling of TCR-antigen binding from generating a CD3 activation signal.

In addition, the engineered TCR-CD3 complex according to the invention may comprise one or more mutation(s) in the alpha chain constant region, in particular one or more of the mutations disclosed below.

The inventors further identified sequence motifs in the constant domain of a TCR which result in decoupling of TCR-antigen binding from the generation of a CD3 activation signal.

### The Sequence Motif PDP

The sequence motif PDP is encoded by the TRAC gene and is thus comprised in the constant domain of the TCR alpha chain. In a particular embodiment, the invention relates to the T cell according to the invention, wherein the at least one mutation in the TCR alpha chain has been introduced into the motif PDP.

In particular, the sequence motif PDP is located at position 4-6 of the constant region of the TCR alpha chain (SEQ ID NO:25. Thus, in certain embodiments, the engineered TCR-CD3 complex according to the invention may comprise one or more mutations in any one of positions 4, 5 and/or 6 of the constant region of the TCR alpha chain (SEQ ID NO:25). In certain embodiments, the engineered TCR-CD3 complex according to the invention comprises a mutation at position P4 (SEQ ID NO:25) of the constant region of the TCR alpha chain. In certain embodiments, the engineered TCR-CD3 complex according to the invention comprises a mutation at position D5 (SEQ ID NO:25) of the constant region of the TCR alpha chain. In certain embodiments, the engineered TCR-CD3 complex according to the invention comprises a mutation at position P6 (SEQ ID NO:25) of the constant region of the TCR alpha chain. In certain embodiments, the engineered TCR-CD3 complex according to the invention comprises mutations at the positions D5 and P6 (SEQ ID NO: 25) of the constant region of the TCR alpha chain. However, it is to be understood that any combination of two or more mutations in the amino acid residues P4, D5 and P6 (SEQ ID NO: 25) of the constant region of the TCR alpha chain may result in an engineered TCR-CD3 complex according to the invention.

In certain embodiments, the invention relates to an engineered TCR-CD3 complex comprising the MART-1 specific TCR DMF5 (SEQ ID NO:51 and 52), wherein the MART-1 specific TCR DMF5 comprises at least one mutation in the sequence motif PDP of the TCR alpha chain (position 114-115 of SEQ ID NO:51). In certain embodiments, the invention relates to an engineered TCR-CD3 complex comprising the NY-ESO-1 specific TCR IG4 (SEQ ID NO:53 and 54), wherein the NY-ESO-1 specific TCR IG4 comprises at least one mutation in the sequence motif PDP of the TCR alpha chain (position 118-120 of SEQ ID NO:53). In certain embodiments, the invention relates to an engineered TCR-CD3 complex comprising the MAGE-A3 specific TCR a3a (SEQ ID NO:55 and 56), wherein the MAGE-A3 specific TCR a3a comprises at least one mutation in the sequence motif PDP of the TCR alpha chain (position 119-121 of SEQ ID NO: 55). In certain embodiments, the invention relates to an engineered TCR-CD3 complex comprising the HCV₁₄₀₆₋₁₄₁₅ specific TCR 1406 (SEQ ID NO:82 and 83), wherein the HCV₁₄₀₆₋₁₄₁₅ specific TCR 1406 comprises at least one mutation in the sequence motif PDP of the TCR alpha chain (position 120-121 of SEQ ID NO:82).

### The Sequence Motif FTDFDS

The sequence motif FTDFDS (SEQ ID NO:90) is encoded by the TRAC gene and is thus comprised in the constant domain of the TCR alpha chain. In a particular embodiment, the invention relates to the T cell according to the invention, wherein the at least one mutation in the TCR alpha chain has been introduced into the motif FTDFDS. In particular, the sequence motif FTDFDS is located at position 24-29 of the constant region of the TCR alpha chain (SEQ ID NO: 25). Thus, in certain embodiments, the engineered TCR-CD3 complex according to the invention may comprise one or more mutations in any one of positions 24, 25, 26, 27, 28 and/or 29 of the constant region of the TCR alpha chain (SEQ ID NO: 25). In certain embodiments, the engineered TCR-CD3 complex according to the invention comprises a mutation at position F24 (SEQ ID NO: 25) of the constant region of the TCR alpha chain. In certain embodiments, the engineered TCR-CD3 complex according to the invention comprises a mutation at position T25 (SEQ ID NO: 25) of the constant region of the TCR alpha chain. In certain embodiments, the engineered TCR-CD3 complex according to the invention comprises a mutation at position D26 (SEQ ID NO: 25) of the constant region of the TCR alpha chain. In certain embodiments, the engineered TCR-CD3 complex according to the invention comprises a mutation at position F27 (SEQ ID NO: 25) of the constant region of the TCR alpha chain. In certain embodiments, the engineered TCR-CD3 complex according to the invention comprises a mutation at position D28 (SEQ ID NO: 25) of the constant region of the TCR alpha chain. In certain embodiments, the engineered TCR-CD3 complex according to the invention comprises a mutation at position S29 (SEQ ID NO: 25) of the constant region of the TCR alpha chain. In certain embodiments, the engineered TCR-CD3 complex according to the invention comprises mutations at the positions F24 and F27 (SEQ ID NO: 25) of the constant region of the TCR alpha chain. However, it is to be understood that any combination of two or more mutations in the amino acid residues F24, T25, D26, F27, D28 and S29 (SEQ ID NO: 25) of the constant region of the TCR alpha chain may result in an engineered TCR-CD3 complex according to the invention.

In certain embodiments, the invention relates to an engineered TCR-CD3 complex comprising the MART-1 specific TCR DMF5 (SEQ ID NO:51 and 52), wherein the MART-1 specific TCR DMF5 comprises at least one mutation in the sequence motif FTDFDS of the TCR alpha chain (position 134-139 of SEQ ID NO:51). In certain embodiments, the invention relates to an engineered TCR-CD3 complex comprising the NY-ESO-1 specific TCR IG4 (SEQ ID NO:53 and 54), wherein the NY-ESO-1 specific TCR IG4 comprises at least one mutation in the sequence motif FTDFDS of the TCR alpha chain (position 138-143 of SEQ ID NO:53). In certain embodiments, the invention relates to an engineered TCR-CD3 complex comprising the MAGE-A3 specific TCR a3a (SEQ ID NO:55 and 56), wherein the MAGE-A3 specific TCR a3a comprises at least one mutation in the sequence motif FTDFDS of the TCR alpha chain (position 139-144 of SEQ ID NO: 55). In certain embodiments, the invention relates to an engineered TCR-CD3 complex comprising the HCV₁₄₀₆₋₁₄₁₅ specific TCR 1406 (SEQ ID NO:82 and 83), wherein the HCV₁₄₀₆₋₁₄₁₅ specific TCR 1406 comprises at least one mutation in the sequence motif FTDFDS of the TCR alpha chain (position 140-145 of SEQ ID NO:82).

### The Sequence Motif FETDxNLN

The sequence motif FETDxNLN (SEQ ID NO:89) is encoded by the TRAC gene and is thus comprised in the constant domain of the TCR alpha chain. Thus, in a particular embodiment, the invention relates to the T cell according to the invention, wherein the at least one mutation in the TCR alpha chain has been introduced into the motif FETDxNLN, wherein x is an undefined amino acid. In particular, the sequence motif FETDxNLN is located at position 103-110 of the constant region of the TCR alpha chain (SEQ ID NO: 25). Thus, in certain embodiments, the engineered TCR-CD3 complex according to the invention may comprise one or more mutations in any one of positions 103, 104, 105, 106, 107, 108, 109 and/or 110 of the constant region of the TCR alpha chain (SEQ ID NO: 25). In certain embodiments, the engineered TCR-CD3 complex according to the invention comprises a mutation at position F103 (SEQ ID NO: 25) of the constant region of the TCR alpha chain. In certain embodiments, the engineered TCR-CD3 complex according to the invention comprises a mutation at position E104 (SEQ ID NO: 25) of the constant region of the TCR alpha chain. In certain embodiments, the engineered TCR-CD3 complex according to the invention comprises a mutation at position T105 (SEQ ID NO: 25) of the constant region of the TCR alpha chain. In certain embodiments, the engineered TCR-CD3 complex according to the invention comprises a mutation at position D106 (SEQ ID NO: 25) of the constant region of the TCR alpha chain. In certain embodiments, the engineered TCR-CD3 complex according to the invention comprises a mutation at position N108 (SEQ ID NO: 25) of the constant region of the TCR alpha chain. In certain embodiments, the engineered TCR-CD3 complex according to the invention comprises a mutation at position L109 (SEQ ID NO: 25) of the constant region of the TCR alpha chain. In certain embodiments, the engineered TCR-CD3 complex according to the invention comprises a mutation at position N110 (SEQ ID NO: 25) of the constant region of the TCR alpha chain. In certain embodiments, the engineered TCR-CD3 complex according to the invention comprises mutations at the positions F103 and E104 (SEQ ID NO: 25) of the constant region of the TCR alpha chain. However, it is to be understood that any combination of two or more mutations in the amino acid residues F103, E104, T105, D106, N108, L109 and N110 (SEQ ID NO: 25) of the constant region of the TCR alpha chain may result in an engineered TCR-CD3 complex according to the invention.

In certain embodiments, the invention relates to an engineered TCR-CD3 complex comprising the MART-1 specific TCR DMF5 (SEQ ID NO:51 and 52), wherein the MART-1 specific TCR DMF5 comprises at least one mutation in the sequence motif FETDxNLN of the TCR alpha chain (position 213-220 of SEQ ID NO:51). In certain embodiments, the invention relates to an engineered TCR-CD3 complex comprising the NY-ESO-1 specific TCR IG4 (SEQ ID NO:53 and 54), wherein the NY-ESO-1 specific TCR IG4 comprises at least one mutation in the sequence motif FETDxNLN of the TCR alpha chain (position 217-224 of SEQ ID NO:53). In certain embodiments, the invention relates to an engineered TCR-CD3 complex comprising the MAGE-A3 specific TCR a3a (SEQ ID NO:55 and 56), wherein the MAGE-A3 specific TCR a3a comprises at least one mutation in the sequence motif FETDxNLN of the TCR alpha chain (position 218-225 of SEQ ID NO:51). In certain embodiments, the invention relates to an engineered TCR-CD3 complex comprising the HCV₁₄₀₆₋₁₄₁₅ specific TCR 1406 (SEQ ID NO:82 and 83), wherein the HCV₁₄₀₆₋₁₄₁₅ specific TCR 1406 comprises at least one mutation in the sequence motif FETDxNLN of the TCR alpha chain (position 219-226 of SEQ ID NO:82).

In certain embodiments, the invention relates to the engineered TCR-CD3 complex according to the invention, wherein the one or more mutation is located at position P4, D5, P6, F24, T25, D26, F27, D28, S29, F103, E104, T105, D106, N108, L109, N110 in the TCR alpha constant region (SEQ ID NO: 25).

Thus, in a particular embodiment, the invention relates to a T cell comprising an engineered TCR-CD3 complex, wherein the engineered TCR-CD3 complex comprises one or more mutations at positions P4, D5, P6, F24, T25, D26, F27, D28, S29, F103, E104, T105, D106, N108, L109 and/or N110 in the TCR alpha constant region (SEQ ID NO: 25), wherein the one or more mutations result in decoupling of TCR-antigen binding from generating a CD3 activation signal.

In a particular embodiment, the invention relates to a T cell comprising an engineered TCR-CD3 complex, wherein the engineered TCR-CD3 complex comprises one or more mutations:
a) at positions W41, Y/F42, R/Q43, Q44, F118, G119, G121, T/S122, V126 and/or P128 (all IMGT) of the TCR alpha chain; and/or
b) at position W41, Y/F42, R/Q43, Q44, F118, G119, G121, T/S122, T125 and/or V/L/T127 (all IMGT) of the TCR beta chain, and/or
c) at positions P4, D5, P6, F24, T25, D26, F27, D28, S29, F103, E104, T105, D106, N108, L109 and/or N110 in the TCR alpha constant region (SEQ ID NO: 25);
wherein the one or more mutations result in decoupling of TCR-antigen binding from generating a CD3 activation signal.

The term “mutation,” as used herein, means or may refer to one or more changes to the sequence of a DNA sequence or a protein amino acid sequence relative to a reference sequence, usually a wild-type sequence. A mutation in a DNA sequence may or may not result in a corresponding change to the amino acid sequence of the encoded protein. A mutation may be a point mutation, i.e. an exchange of a single nucleotide and/or amino acid for another. Point mutations that occur within the protein-coding region of a gene's DNA sequence may be classified as a silent mutation (coding for the same amino acid), a missense mutation (coding for a different amino acid), and a nonsense mutation (coding for a stop which can truncate the protein). A mutation may also be an insertion, i.e. an addition of one or more extra nucleotides and/or amino acids into the sequence. Insertions in the coding region of a gene may alter splicing of the mRNA (splice site mutation), or cause a shift in the reading frame (frameshift), both of which can significantly alter the gene product. A mutation may also be a deletion, i.e. removal of one or more nucleotides and/or amino acids from the sequence. Deletions in the coding region of a gene may alter the splicing and/or reading frame of the gene. A mutation may be spontaneous, induced, naturally occurring, or genetically engineered. Within the present invention, it is preferred that the mutation is a mutation that results in an amino acid exchange.

It is to be understood that the mutations that are required to achieve decoupling of TCR-antigen binding from the generation of a CD3 activation signal may differ between TCRs. In particular, it is to be understood that different mutations in the above-disclosed sequence motifs may be required to achieve decoupling of TCR-antigen binding from the generation of a CD3 activation signal. However, the present invention enables the skilled person to identify whether an engineered TCR-CD3 complex falls within the scope of the present invention.

That is, methods for introducing one or mutations into nucleic acids encoding T cell receptors in a directed fashion are well known in the art. In a first screening step, engineered TCR-CD3 complexes that are efficiently displayed on the cell surface and recognize cognate peptide-MHC complexes may be isolated by flow cytometry. For that, anti-CD3 antibodies, anti-TCR antibodies and/or cognate peptide-HLA-dextramers may be used and are well known in the art. In a second functional screening step, engineered TCR-CD3 complexes falling within the scope of the invention may be identified and isolated. For that, T cells expressing the engineered TCR-CD3 complexes from the first screening round may be contacted with an antigen, such as an antigen-presenting cell, and/or a CD3 agonist and the cells showing the desired levels of T cell activation may be identified and isolated. The criteria for discriminating an engineered TCR-CD3 complex falling within the scope of the invention from a TCR-CD3 complex not falling within the scope of the invention are disclosed elsewhere herein. In view of the teaching provided herein, identifying engineered TCR-CD3 complexes falling within the scope of the invention does not pose an undue burden on the skilled person.

It is to be understood that the present invention does not exclusively relate to T cell comprising an engineered TCR-CD3 complex. In addition, the invention encompasses any of the engineered TCR-CD3 complexes and any of the engineered TCR variants disclosed herein.

### Further Modifications of the Engineered TCR-CD3 Complex

In certain embodiments, the invention relates to the T cell according to the invention, wherein the TCR alpha and/or beta chain further comprise an affinity tag. To increase the safety of pharmaceutical products comprising the T cell according to the invention, the engineered TCR may further comprise an affinity tag which facilitates the recognition and isolation of T cells comprising the engineered TCR-CD3 complex.

The affinity tag may be integrated at any position in the TCR, provided that is does not interfere with correct surface expression of the TCR-CD3 complex. Preferably, the affinity tag is integrated at a surface-accessible position of the TCR, such that it is accessible to antibodies when expressed on the surface of a T cell.

In certain embodiments, the affinity tag may be integrated between the signal peptide and the N-terminus of the TCR alpha and/or beta chain. That is, in a particular embodiment, the invention relates to the T cell according to the invention, wherein the affinity tag is inserted between the signal peptide and the coding sequence of the TCR alpha and/or beta chain.

The term “affinity tag”, as used herein, refers to an amino acid sequence that is used to facilitate purification of a protein or polypeptide. In one embodiment, the affinity tag includes a streptavidin tag, a c-myc tag, an HA-tag, a T7 tag, a FLAG-tag, a polyhistidine tag (such as (His) 6), a polyarginine tag, a polyphenylalanine tag, a polycysteine tag, or a polyaspartic acid tag. Methods of integrating affinity tag into a polypeptide sequence are well established in the art.

### Further Modifications of the T Cell

In a particular embodiment, the invention relates to the T cell according to the invention, wherein the cell further comprises an alloimmune defense receptor (ADR).

ADRs are chimeric antigen receptors that have been disclosed in the art to prevent elimination of allogenic T cells by alloreactive lymphocytes. ADRs that are suitable for use in the T cell of the invention have been disclosed in WO 2019/210081, which is fully incorporated herein by reference.

Further, the T cell may be modified such that it produces cytokines that immunomodulate the tumor environment upon recognition of a tumor antigen. For example, the T cell may be modified such that it produces IL-12 upon recognition of a tumor antigen. IL-12 is known in the art to allow optimal T cell response and to protect T cells from exhaustion.

For example, IL-12 cytokine response elements may be genetically attached to the end domains of the TRBC/TRAC, the CD3 molecules or to the ADR signaling domains. Like this, the secretion of IL-12 from the T cell of the invention may be induced when said T cell interacts with a CD3 agonist, a cognate antigen or a target of the ADR. Preferably, the secretion of IL-12 from the T cell of the invention may be induced when said T cell interacts with a CD3 agonist or a target of the ADR. Constructs for controlling IL-12 expression have been disclosed by Zhang et al. (Molecular Therapy, 2011, 19 (4), p.751-759) and in WO 2010/126766, which is fully incorporated herein by reference.

### Pharmaceutical Compositions

In a particular embodiment, the invention relates to a T cell population comprising a plurality of T cells according to the invention. The T cell population may comprise any number of cells, such as approximately 10³, 10⁴, 10⁵, 10⁶, 10⁷, 10⁸, 10⁹, 10¹⁰, 10¹¹ or more cells. In certain embodiments, substantially all cells in the T cell population comprise the engineered TCR-CD3 complex according to the invention. In certain embodiments, substantially all cells in the T cell population will comprise an identical engineered TCR-CD3 complex. That is, substantially all TCR-CD3 complexes comprised in the cells of the T cell population can interact with the same peptide-MHC complex.

In a particular embodiment, the invention relates to a pharmaceutical composition comprising the T cell according to the invention or a T cell population according to the invention.

To facilitate administration, the T cells according to the invention can be made into a pharmaceutical composition for administration in vivo, with appropriate carriers or diluents, which further can be pharmaceutically acceptable. The means of making such a composition have been described in the art (see, for instance, Remington's Pharmaceutical Sciences, 16th Ed., Mack, ed. (1980)). Where appropriate, the T cells can be formulated into a preparation in semisolid or liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. Desirably, however, a pharmaceutically acceptable form is employed that does not in effectuate the cells expressing the engineered TCR-CD3 complex. Thus, desirably the T cells can be made into a pharmaceutical composition containing a balanced salt solution, preferably Hanks' balanced salt solution, or normal saline.

In a particular embodiment, the invention relates to the pharmaceutical composition according to the invention further comprising a CD3 agonist. That is, the pharmaceutical composition may further comprise any one of the CD3 agonists disclosed herein. Preferably, the CD3 agonist is a CD3-bispecific antibody. Preferably, the CD3-bispecific antibody can bring the T cell of the invention in close proximity with a tumor cell or a virus-infected cell.

In a particular embodiment, the invention relates to a kit-of-parts comprising the T cell according to the invention and a CD3 agonist. Preferably, the CD3 agonist is any of the CD3 agonists disclosed herein, preferably any of the multispecific CD3 agonists disclosed herein.

The pharmaceutical composition according to the invention may comprise further therapeutic agents, such as additional anticancer drugs or antiviral agents. Further, the pharmaceutical composition may comprise additional agents that augment an immune response (for treatment of cancer or viral infections) or suppress an immune response (for treatment of autoimmune diseases).

### Therapeutic Uses

In a particular embodiment, the invention relates to the T cell according to the invention, the T cell population according to the invention or the pharmaceutical composition according to the invention for use as a medicament.

That is, the T cell according to the invention, or compositions comprising the T cell according to the invention, may be used in the treatment of various medical conditions.

In certain embodiments, the T cell according to the invention, or compositions comprising the T cell according to the invention, may be used in the treatment of cancer. The cancer may be any type of cancer. T cells play an important role in the elimination of cancerous cells. It is thus plausible that the T cells according to the invention, or compositions comprising the T cells according to the invention, can be used in the treatment of any type of cancer.

In certain embodiments, the T cells according to the invention may be used in the treatment of hematological malignancies. As used herein, the term “hematological malignancies” relates to myeloid hematological malignancies and lymphoid hematological malignancies. As used herein, myeloid hematological malignancies and lymphoid hematological malignancies also include pre-malignant myeloid or lymphoid hematological disorders and non-neoplastic or non-malignant myeloproliferative or lymphoproliferative disorders.

In particular, the T cells of the present invention may be used for the treatment or prevention of:

Myeloid hematological malignancies, such as acute myeloid leukemia (AML) (e.g. Erythroleukemia, acute megakaryoblastic leukemia, Acute eosinophilic leukemia, Acute basophilic leukemia, Acute myelomonocytic leukemia, acute myeloblastic leukemia); Chronic myelogenous leukemia; Myelodysplasic syndrome; Chronic myelomonocytic leukemia; and Myeloproliferative diseases (e.g. myelofibrosis, acute biphenotypic leukemia, Polycythemia vera, Chronic eosinophilic leukemia/Hypereosinophilic syndrome, Essential thrombocytosis, and Chronic eosinophilic leukemia/Hypereosinophilic syndrome).

Lymphoid Hematological malignancies, such as acute lymphoblastic leukemia (ALL), T-cell lymphoblastic leukemia/lymphoma.

In a particularly preferred embodiment, the T cells of the invention are used in the treatment of B cell malignancies. The term “B-cell malignancy” or “B-cell neoplasm” in its broadest sense refers to a malignancy or neoplasm of B cells, i.e. derived from any stage of a B cell. The term encompasses B-cell lymphomas, B-cell leukemias, and myelomas. A B-cell malignancy in accordance with the present invention is characterized by the presence of malignant B-cells, preferably clonal malignant B-cells expressing a BCR. “Malignant” cells are generally not self-limited in their growth, are capable of invading into adjacent tissues, and may be capable of spreading to distant tissues (metastasizing). “Malignant” when used herein is synonymous with “cancerous”. When used herein, the term “malignant B-cell” is in particular envisaged to refer to a B-cell that can evade apoptosis, displays self-sufficiency of growth signals, and/or exhibits insensitivity to antigrowth signals, as ascertainable using routine methods known in the art.

In certain embodiments, the T cells of the invention may be used in the treatment of solid tumors. “Solid Tumor” as used herein includes cancerous and noncancerous solid tumors. Cancerous solid tumors include, without limitation, biliary tract cancer; brain cancer, including glioblastomas and medulloblastomas; breast cancer; cervical cancer; choriocarcinoma; colon cancer; endometrial cancer; esophageal cancer; gastric cancer; intraepithelial neoplasms, including Bowen's disease and Paget's disease; liver cancer; lung cancer; lymphomas, including Hodgkin's disease and lymphocytic lymphomas; neuroblastomas; oral cancer, including squamous cell carcinoma; ovarian cancer, including those arising from epithelial cells, stromal cells, germ cells and mesenchymal cells; pancreas cancer; prostate cancer; colorectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma [teratomas, choriocarcinomas]), stromal tumors and germ cell tumors; thyroid cancer, including thyroid adenocarcinoma and medullar carcinoma; and renal cancer including adenocarcinoma and Wilms tumor, but excludes tumors of non-solid tissues such as leukemias and other hematological neoplasms, including acute lymphocytic and myelogenous leukemia; multiple myeloma; AIDS associated leukemias and adult T-cell leukemia lymphoma.

In a particular embodiment, the invention relates to the T cell according to the invention, the T cell population according to the invention or the pharmaceutical composition according to the invention for use in the treatment of a viral infection. T cells play an important function in the elimination of virus infected cells. It is thus plausible that the T cells according to the invention, or compositions comprising the T cells according to the invention, can be used in the treatment of any type of viral infection.

The term “viral infection” as used herein, is broadly defined as an infection that is caused by a virus, either an RNA virus or a DNA virus. In some embodiments the viral infection is by a virus from the following families of viruses: Orthomyxoviridae (e.g. influenza A virus), Paramyxoviridae (e.g. measle virus, Sendai virus, Newcastle disease virus), Flaviviridae (e.g. hepatitis C virus, Japanese encephalitis virus, West Nile virus, Dengue virus), Reoviridae (e.g. reovirus), Rhabdoviridae (e.g. rabies virus, vesicular stomatitis virus), and adenoviruses. In some embodiments the virus is selected among influenza A virus, Sendai virus, hepatitis C virus (HCV), rabies virus, Japanese encephalitis viruses, and herpes simplex virus. In some embodiments the viral infection is by a virus that causes domestic animals diseases, including but not limited to the Newcastle disease virus (NDV) and Sendai virus which infect all known (wild and domestic) bird species. In certain embodiments, the viral infection is caused by SARS-CoV-2.

In a particular embodiment, the invention relates to the T cell according to the invention, the T cell population according to the invention or the pharmaceutical composition according to the invention for use in the treatment of an autoimmune disease. The T cells according to the invention may be used for attacking autoreactive immune cells. It is thus plausible that the T cells according to the invention can be used in the treatment of autoimmune diseases.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia greata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

The T cells of the invention may be used as a single therapeutic agent or as part of a combination therapy. Administering the T cell of the invention with a CD3 agonist offers the potential to control the activity of the T cell according to the invention. Thus, it is preferred that the T cell according to the invention is administered together with a CD3 agonist.

In a particular embodiment, the invention relates to the T cell, the T cell population or the pharmaceutical composition for use according to the invention, wherein the human T cell is administered before, concomitantly or after a CD3 agonist.

That is, in certain embodiments, the T cell according to the invention may be administered prior to the CD3 agonist. For example, the T cell according to the invention may be administered 1 hour, 2 hours, 4 hours, 8 hours, 12 hours, 1 day, 2 days, 3 days, 4 days, 5 days, 6 days or 7 days prior to the CD3 agonist.

In certain embodiments, the CD3 agonist may be administered prior to the T cell according to the invention. For example, the CD3 agonist may be administered 1 hour, 2 hours, 4 hours, 8 hours, 12 hours, 1 day, 2 days, 3 days, 4 days, 5 days, 6 days or 7 days prior to the T cell according to the invention.

In certain embodiments, the T cell according to the invention may be administered at the same time as the CD3 agonist. In certain embodiments, the T cell according to the invention may be administered once to a subject in need and the CD3 agonist is administered multiple times. For example, the CD3 agonist may be administered 1, 2, 3, 4 or 5 times after the administration of the T cell according to the invention.

In certain embodiments, both the T cell according to the invention and the CD3 agonist may be administered multiple times to a subject in need.

For any of the administration schemes disclosed above, it is to be understood that the exact administration scheme depends on the various factors, including the medical indication to be treated, the response of the patient to the treatment, the medical history of the patient, and so forth. Further, dose finding studies are commonly performed in the art to identify a therapeutically effective amount for a given patient or a given medical indication.

The term “therapeutically effective amount” as used herein means that the number of T cells of the present invention, and optionally the amount of a CD3 agonist, contained in the composition administered is of sufficient quantity to achieve the intended purpose, such as, in this case, to treat cancer, a viral infection or an autoimmune disease. The skilled person is aware of methods to determine whether a treatment has the desired therapeutic effect.

To allow a targeted treatment of any of the diseases or disorders disclosed above, it is preferred herein that the T cells of the invention are administered together with a targeting agent. Preferably, the targeting agent is a bi- or multispecific binding agent, such as a bi- or multispecific antibody or an ImmTAC.

In a preferred embodiment, the T cell according to the invention is administered together with a CD3-bispecific antibody or ImmTAC. CD3-bispecific antibodies or ImmTACs have the ability to activate the T cell of the invention and thereby induce an immune response. In addition, CD3-bispecific antibodies or ImmTACs can bind to a specific target antigen and thereby bring the T cell according to the invention into close proximity with a target cell. The target antigen that is bound by the CD3-bispecific antibody or ImmTAC may preferably by an antigen that is present on cancerous cells, virus-infected cells or autoreactive immune cells. As such, combining the T cell according to the invention with a bi- or multispecific binding agent offers the potential to improve the treatment of cancer, viral infections and autoimmune diseases.

In a particular embodiment, the invention relates to the T cell, the T cell population or the pharmaceutical composition for use according to the invention, wherein the T cell has been obtained from a patient to be treated.

That is, the T cell used in the treatment of cancer, viral infections or autoimmune diseases may be an autologous T cell. For that, the T cell may be isolated from a patient, genetically engineered ex vivo, and reintroduced back into the same patient, preferably together with a CD3 agonist.

In a particular embodiment, the invention relates to the T cell, the T cell population or the pharmaceutical composition for use according to the invention, wherein the T cell has been obtained from a donor.

The T cells of the invention have the advantage that binding of to a cognate peptide-MHC complex alone is not sufficient to induce an immune response. Due to this characteristic, the T cells of the present invention are particularly well suited for use in allogenic T cell therapy, as they have a significantly reduced risk of attacking the recipient's body's cells. Thus, using the T cell of the invention significantly reduces the risk of graft-versus-host disease in allogenic T cell therapy. T cells of the invention may thus be used as off-the shelf T cells that can be prepared in advance at a centralized facility, thereby significantly reducing the costs of cell therapy.

The donor of the T cell is preferably a healthy donor. Methods for obtaining T cells from a donor are known in the art and include apheresis. T cells may be isolated from a donor's blood and subsequently genetically engineered to obtain the T cell according to the invention. However, the T cells of the invention may also be obtained by isolating hematopoietic stem cells (HSC) or hematopoietic stem and progenitor cells (HSPC) from a donor which are then differentiated into T cells ex vivo. Alternatively, the T cells of the invention may be obtained by isolating somatic cells from a donor which are first converted into induced pluripotent stem cells (IPSC) and then differentiated into T cells.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermaliy, intratumorally, intranodally, intramedullary, intramuscularly, by intravenous or intralymphatic injection, or intraperitoneally. In one embodiment, the cell compositions of the present invention are preferably administered by intravenous injection. The administration of the cells or population of cells may consist of the administration of 10⁴-10⁹ cells per kg body weight. The cells or population of cells may be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, said effective amount of cells or composition comprising those cells are administrated parenterally. Said administration can be an intravenous administration. Said administration can be directly done by injection within a tumor.

The term “treat” as used herein refers to any type of treatment that imparts a benefit to a patient afflicted with a disease, including improvement in the condition of the patient (e.g., in one or more symptoms), delay in the progression of the condition, etc. The term “patient” as used herein refers to human or animal subjects. As used herein, the term “subject” refers to an animal, particularly a mammal, particularly a human.

### Methods for Producing the T Cell of the Invention

The invention further encompasses methods for producing the T cell according to the invention.

Thus, in a particular embodiment, the invention relates to a method for generating a T cell according to the invention, the method comprising the steps of:
(i) introducing at least one mutation into a TCR alpha and/or beta chain of a T cell that has been obtained from a donor; and
(ii) obtaining a T cell comprising and engineered TCR-CD3 complex.

In a particular embodiment, the invention relates to a method according to the invention, wherein the at least one mutation in the TCR alpha and/or beta chain is introduced outside of a complementary determining region (CDR).

In a particular embodiment, the invention relates to a method according to the invention, wherein the at least one mutation in the TCR alpha and/or beta chain is introduced at the interface between the TCR alpha and beta chain.

In a particular embodiment, the invention relates to a method according to the invention, wherein the at least one mutation in the TCR alpha and/or beta chain results in reduced TCR alpha and beta association.

In a particular embodiment, the invention relates to a method according to the invention, wherein the at least one mutation in the TCR alpha and/or beta chain is introduced in the sequence motif WYRQ (IMGT position 41-44), FG₁xG₂T (IMGT position 118-122), VxP (IMGT position 126-128), PDP (position 4-6 of TCR alpha constant region (SEQ ID NO:25)), TDFDS (position 24-29 of TCR alpha constant region (SEQ ID NO:25)) and/or FETDxNLN (position 103-110 of TCR alpha constant region (SEQ ID NO:25)), wherein x is an undefined amino acid.

In a particular embodiment, the invention relates to a method according to the invention, wherein at least one of the amino acid residues G₁ and/or G₂ in the motif FG₁xG₂T (IMGT position 118-122) is replaced with another amino acid.

In a particular embodiment, the invention relates to a method according to the invention, wherein the motif FG₁xG₂T in the TCR alpha and/or beta chain is replaced with the sequence FEQWT.

In a particular embodiment, the invention relates to a method according to the invention, wherein the at least one mutation in the TCR alpha and/or beta chain is introduced:
at position W41, Y/F42, R/Q43, Q44, F118, G119, G121, T/S122, V126 and/or P128 of the variable domain of the TCR alpha chain (according to IMGT numbering); and/or
at position W41, Y/F42, R/Q43, Q44, F118, G119, G121, T/S122, T125 and/or V/L/T127 of the variable domain of the TCR beta chain (according to IMGT numbering); and/or
at position P4, D5, P6, F24, T25, D26, F27, D28, S29, F103, E104, T105, D106, N108, L109, N110 of the TCR alpha constant region (SEQ ID NO:25).

In a particular embodiment, the invention relates to a method for generating a decoupled T cell receptor, the method comprising the steps of:
i) introducing at least one mutation into a TCR alpha and/or beta chain of a T cell receptor,
   wherein the at least one mutation in the TCR alpha and/or beta chain is introduced in the sequence motif WYRQ (IMGT position 41-44), FG₁xG₂T (IMGT position 118-122), VxP (IMGT position 126-128), PDP (position 4-6 of TCR alpha constant region (SEQ ID NO:25)), TDFDS (position 24-29 of TCR alpha constant region (SEQ ID NO:25)) and/or FETDxNLN (position 103-110 of TCR alpha constant region (SEQ ID NO:25)), wherein x is an undefined amino; and/or
   wherein the at least one mutation in the TCR alpha and/or beta chain is introduced:
   a) at position W41, Y/F42, R/Q43, Q44, F118, G119, G121, T/S122, V126 and/or P128 of the variable domain of the TCR alpha chain (according to IMGT numbering); and/or
   b) at position W41, Y/F42, R/Q43, Q44, F118, G119, G121, T/S122, T125 and/or V/L/T127 of the variable domain of the TCR beta chain (according to IMGT numbering); and/or
   c) at position P4, D5, P6, F24, T25, D26, F27, D28, S29, F103, E104, T105, D106, N108, L109, N110 of the TCR alpha constant region (SEQ ID NO:25); and
ii) obtaining a decoupled T cell receptor.

In a particular embodiment, the invention relates to a method according to the invention, wherein the T cell is a human T cell.

In a first step, one or more of the mutations disclosed herein are introduced into the TCR alpha and/or beta chain of a T cell that has been obtained from a donor. As mentioned above, a T cell may be obtained from a donor by isolating the desired type of T cell from the blood of a donor. Alternatively, cells may be differentiated ex vivo from stem cells (HSC, HSPC, IPSC) that have been obtained from a donor (HSC, HSPC) or generated from cells that have been obtained from a donor (IPSC). The skilled person is aware of methods to obtain T cells from a donor for any of the procedures mentioned above.

Introducing one or more mutations in a TCR alpha or beta chain is preferably achieved by introducing one or more mutations into a nucleic acid molecule encoding said TCR alpha or beta chain. Preferably, the mutations in the TCR nucleic acid molecule result in an amino acid exchange in the TCR alpha or beta chain. The skilled person is aware of methods for introducing mutations into a nucleic acid molecule encoding a TCR alpha or beta chain. Preferably, mutations are introduced into a nucleic acid by genome editing.

Thus, in a particular embodiment, the invention relates to the method according to the invention, wherein the at least one mutation is introduced by genome edition.

The term “genome editing”, as used herein refers to altering one or more nucleotides within the genome of a cell. The cell may be in vivo. The cell may be ex vivo or in vitro. Non-limiting examples of genome editing methods include CRISPR-mediated genetic modification polypeptides such as Cas9, Cas12a (Cpf1), or other CRISPR endonucleases, Argonaute endonucleases, transcription activator-like (TAL) effector and nucleases (TALEN), zinc finger nucleases (ZFN), expression vectors, transposon systems (e.g., PiggyBac transposase), or any combination thereof. Designer zinc fingers, transcription activator-like effectors (TALEs), or homing meganucleases are available for producing targeted genome perturbations.

In a particular embodiment, the invention relates to the method according to the invention, wherein the genome editing step involves the use of a CRISPR-Cas system. That is, the mutation in the TCR alpha and/or beta chain are preferably introduced by one of the CRISPR/CAS systems disclosed above.

Alternatively to genome editing, one or more mutations may be introduced into a TCR alpha and/or beta chain of a T cell by viral transduction. For example, a construct encoding a mutated TCR alpha and/or beta chain may be introduced into a T cell by viral transduction. The skilled person is aware of viral vectors that are suitable for the transduction of T cells, such as lentiviral vectors or AAV vectors. In such embodiments, the endogenous TCR of the T cell may be inactivated by genome editing. For example, a viral vector may be used to introduce an endonuclease, such as Cas9, into the T cell. Subsequently, the T cell may be transfected with a suitable RNA to knock out one or more elements of the endogenous TCR. Like this, T cells can be obtained that only express the engineered TCR-CD3 complex according to the invention.

In a particular embodiment, the invention relates to the method according to the invention further comprising a step of introducing an affinity tag into the TCR alpha and/or beta chain of the T cell.

Introducing an affinity tag into a TCR alpha and/or beta chain is preferably achieved by integrating a nucleic acid encoding said affinity tag into a nucleic acid encoding a TCR alpha or beta chain. Integration of the nucleic acid encoding the affinity tag into a nucleic acid encoding a TCR alpha or beta chain may be achieved by methods commonly known in the art. Preferably, introducing an affinity tag into a TCR alpha and/or beta chain is achieved by genome editing, in particular by any one of the genome editing methods disclosed herein.

Generating the T cell according to the invention may comprise one or more functional assays to verify that the T cell is a T cell falling within the scope of the invention. That is, the method of generating a T cell according to the invention may comprise a first functional assay in which the correct surface expression of the engineered TCR-CD3 complex and the antigen-binding capacity of the TCR are confirmed. As disclosed above, surface expression may be analyzed by flow cytometry with an anti-CD3 and/or an anti-TCR antibody. Antigen binding may be analyzed by flow cytometry with a suitable MHC dextramer.

In a second functional assay, it may be confirmed whether the engineered TCR is a decoupled TCR as defined herein. For this, the level of T cell activation in response to an antigen and/or a CD3 agonist may be determined. The antigen may be presented by an antigen-presenting cell and the CD3 agonist may be any one of the CD3 agonists disclosed herein. The level of T cell activation may be determined by quantifying the secretion of IL-2 and/or IFNγ or by quantifying the proliferation rate of the T cells.

### BRIEF DESCRIPTION OF THE DRAWINGS

FIG. 1. Mutations in the aCPM of TCRs abrogate TCR-antigen binding and CD3-signaling a. The TCR-CD3 complex is a functionally dependent octamer; genomic knock-out (KO) of any of the components disrupts a complete assembly in the endoplasmic reticulum (ER) and Golgi apparatus. Individual chains and incomplete TCR complexes are retained or degraded in the ER and the end result is loss of surface expression of the entire complex; for example, CRISPR/Cas9 KO of the TCR alpha chain constant region (TRAC) leads to a functional KO of CD3 molecules. b. The alpha chain connecting peptide motif (aCPM) has been identified as a key motif for TCR-antigen (peptide-MHC) binding and CD3 signal transduction in mouse T cells. The motif is spanning the junction of exon 2 and 3 of the TRAC locus and its residues (FETDxNLN) are highly conserved across mammalian species. Two Jurkat cell lines expressing TCR variants with mutations in aCPM (Jkt-DMF5_{FATADALN} (SEQ ID NO:27) and Jkt-DMF5_{GGGSGSG} (SEQ ID NO:28) are shown. c. Schematic representation of the co-culture assays. Antigen presenting cells (T2) are pulsed with a different concentration of peptide (ELA) and cultured overnight with Jkt-DMF5 cells in a 1:2 ratio; Raji cells express CD19 antigen and are cultured with blinatumomab and Jkt-DMF5 overnight in 1:2 ratio. d. Left panel, overnight co-cultures with the T2 cells pulsed with MART-1 peptide antigen [ELAGIGILTV (ELA; SEQ ID NO:62)]. Right panel, Jkt-T cell NFAT-GFP dose response to blinatumomab (ng/ml) in co-culture with Raji (CD19+) tumor cells. Data is normalized to the Jkt-DMF5 WT. e. The panel shows a comparison of the highest responses of all TCR variants to peptide (left) and blinatumomab (right) with statistical analysis. f. Representative flow cytometry plots of T cell binding to MART-1 dextramer and CD3 expression reveals a reduction of TCR/CD3 surface expression in Jkt-DMF5_{FATADALN} and Jkt-DMF5_{GGGSGSG} variants. g. Assembly of the extracellular domains (ECD) of the TCR/CD3 complex is mediated by the connecting peptides (CP) of the TCR αβ chains and their molecular interactions with CD3γε and CD3εδ. Multiple bonds between TCR-α cPM residues (F236-N243) and CD3 molecules are displayed (PDB: 6JXR). Asterisks indicate statistical significance between Jkt-DMF5 variants and Jkt-DMF5 WT TCR as determined by one-way ANOVA with Tukey's post hoc test for multiple comparisons. Data are displayed as mean±SD. *P<0.05, **P<0.01, ***P<0.001, ****P<0.0001, ns=not significant. h. The CDR3 region of the Jurkat T-cell β chain is targeted with a specific sgRNA. LHA and RHA (Left and Right homology arms), SA (splice acceptor), complete a-DMF5 chain, P2A coding sequence, VID-B-DMF5 chain containing the splice donor (SD) which splices with the endogenous TRBC. The entire homology-directed repair (HDR) template is 3111 bp long and is PCR-amplified with the F1 (GCATGGATCCCAATGC, SEQ ID NO:84) and R1 (TTTTATCTGTCATGGCCGTGACCG, SEQ ID NO:85) primers.

FIG. 2. TCR sequence analysis and functional decoupling of TCR-antigen binding from CD3-signaling. a. Key criteria defined for identifying motifs to enable decoupling of TCR-antigen binding from CD3-signaling. b. Multiple sequence alignment of TCRa J-gene germlines (TRAJ) from human (left; SEQ ID NO:98-118) and selected mammals (right) shows a highly conserved motif (FGxGT). c. DMF5 TCR (PDB: 3QDJ) structure is represented with alpha-beta chain spatial conformation and CDRs. The magnified square depicts inter-chain molecular contacts between the α-chain FGxGT motif and β-chain residues in proximity. Complete amino acid sequence of the DMF5 TCR chains (SEQ ID NO:51 and 52) with highlighted CDRs is located in the top-right corner. d. Representative flow cytometry plots of NFAT-GFP and CD3 expression in Jkt-DMF5 and Jkt-AED_{DMF5 01} cells. T cells were co-cultured overnight with peptide pulsed T2 cells (ELA). In grey, Jkt-CD3⁻ cell-line basal activation and in culture with the highest concentration of ELA peptide (10 μg/mL). e. Left panel, NFAT-GFP response curves for Jkt-DMF5 and Jkt-AED_{DMF5 01} cells to three known cognate peptide antigens (ELA, AAG, EAA). Right panel, CD3 activation curves with blinatumomab. Data normalized to the Jkt-DMF5. f. Representative flow plots of MART-1-HLA A2 dextramer binding. g. Left panel, bar plots of two highest peptide concentrations 4 log 10 (nM) (10 μg/mL) and 3 log 10 (nM) (1 μg/mL) measured for Jkt-DMF5 and Jkt-AED_{DMf5 01} and their statistical analysis. Right panel, selected two highest concentrations for blinatumomab. Data is displayed as mean±SD and each dot is an individual data point. P-values were determined using two-tailed, unpaired Student's t-test. *P<0.05, **P<0.01, ***P<0.001, ****P<0.0001, ns=not significant. h. 1G4 TCR (SEQ ID NO:53 and 54) is specific for HLA A2*01 MHC and SLLMWITQC peptide (SEQ ID NO:63). On the right, a close-up of the FGxGT motif and its interaction with the corresponding amino acids in the β-chain is shown. F104 (α-chain) forms multiple bonds with the adjacent amino acids in the β-chain. i. a3a TCR (SEQ ID NO:55 and 56) is specific for HLA A01*01 and EVDPIGHLY peptide (SEQ ID NO:64). The panel consists of the same elements as the panel a and the close-up highlights the F105 conserved interactions with the a3a β-chain

FIG. 3. AED T cell discovery with library mutagenesis and functional screening. a. Top: Shown are three TCRs with specificity to tumor associated antigens and their HLA-restriction, TCRa V-gene germline (TRAV) and TRAJ usage and FGxGT sequence motif (SEQ ID NO:57-59). Middle: Table depicting the TCRs used in the library screening, the α- and β-chain composition and HLA-peptide specificity. Bottom: Representative flow cytometry plots of library selection rounds. Peptide negative fraction was selected as a starting Jkt-population for the following round. Each TCR library was enriched 3 times. Peptide concentration of 100 ng/ml was used as a threshold to separate high- and low-response T cell populations. Blinatumomab was used at the concentration of 12 ng/ml. b. The DMF5 motif (FGQGT) was used for all the libraries. The glycine (G) residues were mutated with NNK mutagenic oligonucleotides yielding a library size of 400 variants for each TCR. First T cells with maintained CD3 surface expression and pMHC binding were sorted by FACS. Next, co-culture assays were performed with either T2 cells pulsed with cognate peptide (DMF5 and 1G4) or with EJM cells (a3a); both high- and low-NFAT-GFP expressing variants isolated by FACS. The same selection strategy was applied to cells co-cultured with CD19+ cells and blinatumomab. The fraction with a low response to the peptide (orange square) was pursued in the next round of selection (three rounds total). Genomic DNA from each group was extracted and submitted for deep sequencing. Sequencing analysis of variants deselected in the peptide bracket (P2) and with high-performance in the blinatumomab bracket (P3) were chosen for further analysis. c. A heat map representation of selected FGxGT motif variants (SEQ ID NO:9-21 and 70-81) and a number of TCR variants that are not fulfilling one or more criteria (groups separated with a dashed line). An appropriate AED candidate must be present in the CD3/pHLA group, underrepresented in peptide+ (PEP+) group and overrepresented in the blinatumomab+ group (BLINA+). d. Library selected variants were introduced into Jurkat T cells and individually tested for peptide response. Several variants were introduced into the 1406 TCR as well. e. Response to blinatumomab stimulation (12 ng/ml) of each AED candidate and their designated WT TCRs. Data was normalized to the highest observed value in the TCR group and is displayed as the mean±SD. P values were determined using a one-way ANOVA with Tukey's correction for multiple comparison. *P<0.05, **P<0.01, ***P<0.001, ****P<0.0001, ns=not significant.

FIG. 4. In vitro functional assays of primary human AED T cells. a. Primary human T cells (AED_{DMF5 01} and WT_{DMF5}) were transfected with the AAV-CRISPR-Cas9 and isolated by FACS based on binding to MART-1 dextramer CD3 expression b. T cells were co-cultured with T2 cells pulsed with a range of cognate peptides (ELA, AAG, EAA). Flow cytometry plots represent T cell proliferation to varying ELA peptide concentration. Percentages of CD19+ (T2-peptide pulsed) and CD3+ cells (T cells) are given in the Figures. The starting ratio on day 0 for each condition was 10:1 (T2: T-cells). c. The proliferation curves for AED_{DMF5 01} and WT_{DMF5} are plotted for each MART-1 peptide and three different HLA donors. d. Co-culture supernatants were harvested 24 h and 120 h after the start of the experiment. Three different donors (UDN001, UDN002, UDN003) were used. Values of IL-2 concentration in the supernatant after 24 h were measured by human IL-2 ELISA and dose response curves are displayed. e. IL-2 and IFN-γ measured values for the highest peptide concentration (log 104 (nM)) are plotted. TCR pairs (AED_{DMF5 01} and WT_{DMF5}) of three peptide antigens (ELA, AAG, EAA) for each donor are stacked together with individual data points shown on each graph f. The IFN-γ concentration fold changes from supernatants collected on day one and five are shown. g. T-cell proliferation, IL-2 and IFN-γ secretion are shown for WT UDN, WT_{DMF5} and AED_{DMF5 01} T cells across three donors following co-culture with CD19+ tumor cells (Raji) and blinatumomab. h. Raji cells were labeled with a red fluorescent dye and were added to the wells containing T cells at a 1:5 ratio. Microscopy images depict the Raji cell cluster size difference between −/+blinatumomab samples and a formation of T cell rings is observed around Raji cells when blinatumomab is added. In all the bar plots, data is displayed as the mean±SD. For e and f P-values were calculated using a two-tailed unpaired Student's t test. g. P values were determined with one-way ANOVA with Tukey's correction for multiple comparisons *P<0.05, **P<0.01, ***P<0.001, ****P<0.0001, ns=not significant. h. Raji cells were labeled with a red fluorescent dye and were added to the wells containing T cells at a 1:5 ratio. Microscopy images depict the Raji cell cluster size difference between −/+ blinatumomab samples and a formation of T cell rings is observed around Raji cells when blinatumomab is added. i. Cas9-mediated integration was performed with the sgRNA targeting the exon1 of the TRAC locus. The complete DMF5 TCR was introduced in frame with the endogenous TRAC locus by using the AAV6-plasmid (p77-DMF5). j. FACS plots representing CD3+ and CD3/dex+ fractions in WT DMF5 and AED_{DMF5 01} T-cells. k. Representative flow cytometry (or FACS) plots display the uneven distribution of CD4+ T cells between the UDN011 and genetically engineered DMF5 T cells (03-DMF5 and 03-AED_{DMF5 01}). Bar plot shows the distribution of CD4+ and CD8+ T cells across donors and their engineered variants. Numbers plotted represent the CD8+ fraction.

FIG. 5. AED T-cells with blinatumomab drive potent anti-tumor response and do not display alloreactivity. a. Schematic of the human tumor xenograft mouse model. All cell injections were performed subcutaneously with a 1:15 Raji/T-cell mix. WT T-cells were a 1:1 mix of 2 donors (UDN001 and UDN002). Blinatumomab (0.1 μg) was administered intravenously once a day for five consecutive days. Mice were sacrificed on day 42 when the control group (Raji only) reached the highest allowed level of luciferase (5×10⁹ photons/s). b. Bioluminescence activity in each mouse group measured on day 0 by IVIS imaging. c. Tumor progression was followed with weekly IVIS imaging at specified time points. Four mice in each group with WT T cells were sacrificed on day 28 to determine the complete loss of bioluminescence. d Tumor bioluminescence measured at the end of experiment (day 42) e. Tumor bioluminescence signals measured at specified time points. Individual lines denote data obtained from each animal. f. Kaplan-Meier curves showing overall survival of mice in the selected experimental groups. P-Values were determined using a two-sided log rank-test. g. Immunohistochemistry (CD19) and chromogenic stainings (CD3) of representative mice in the mouse groups with tumor cell overgrowth. Data are displayed as mean±SD. b, P values were calculated using one-way ANOVA with Sidak's correction for multiple comparison *P<0.05, **P<0.01, ***P<0.001, ****P<0.0001, ns=not significant.

FIG. 6. AED-modified Jurkat T cells are no longer activated by a melanoma cell line.

FIG. 7. AED TCR-reconstituted primary T cells express reduced levels of Granzyme B in response to a cognate antigen.

FIG. 8. AED TCR-reconstituted primary T cells express normal levels of Granzyme B in response to a CD3 agonist.

FIG. 9. AED TCR-reconstituted primary T cells express reduced levels of LAMP-1 in response to a cognate antigen.

FIG. 10. AED TCR-reconstituted primary T cells express normal levels of LAMP-1 in response to a CD3 agonist.

FIG. 11. AED-modified Jurkat T cells are activated by various bispecific anti-CD antibodies.

FIG. 12. Each dataset represents selected variants from a WYRQ motif library that are present in the bulk (CD3+ fraction) and in GFP+ fraction upon selection with blinatumomab (SEQ ID NO:149-162). The variants are also deselected in the GFP+ fraction upon peptide stimulation. Each Library selected variants are represented in the Fold Enrichment figure (up) and total count figure (down) and color code represents number of mutations in the variants compared to the WT sequence.

FIG. 13. Each dataset represents selected variants from a VxP motif library that are present in the bulk (CD3+ fraction) and in GFP+ fraction upon selection with blinatumomab. The variants are also deselected in the GFP+ fraction upon peptide stimulation. Each Library selected variants are represented in the Fold Enrichment figure (up) and total count figure (down) and color code represents number of mutations in the variants compared to the WT sequence.

FIG. 14. Each dataset represents selected variants from a FTDFDS motif library that are present in the bulk (CD3+ fraction) and in GFP+ fraction upon selection with blinatumomab (SEQ ID NO:163-167). The variants are also deselected in the GFP+ fraction upon peptide stimulation. Each Library selected variants are represented in the Fold Enrichment figure (up) and total count figure (down) and color code represents number of mutations in the variants compared to the WT sequence.

FIG. 15. Each dataset represents selected variants from a PDP motif library that are present in the bulk (CD3+ fraction) and in GFP+ fraction upon selection with blinatumomab. The variants are also deselected in the GFP+ fraction upon peptide stimulation. Each Library selected variants are represented in the Fold Enrichment figure (up) and total count figure (down) and color code represents number of mutations in the variants compared to the WT sequence.

FIG. 16. Each dataset represents selected variants from a FETDxNLN motif library that are present in the bulk (CD3+ fraction) and in GFP+ fraction upon selection with blinatumomab (SEQ ID NO:168-186). The variants are also deselected in the GFP+ fraction upon peptide stimulation. Each Library selected variants are represented in the Fold Enrichment figure (up) and total count figure (down) and color code represents number of mutations in the variants compared to the WT sequence.

### EXAMPLES

### Example 1

Herein, the inventors report the engineering of Allogeneic-Engineered-Decoupled (AED) T cells: allogeneic T cells with TCR-antigen binding decoupled from CD3 signaling, all while maintaining a functional TCR-CD3 cell surface expression. Through TCR germline sequence and structural analyses, the inventors identified highly conserved sequence motifs across human and other mammalian species. By performing targeted genomic mutagenesis, functional screening and deep sequencing in the newly discovered motifs, the inventors engineered novel TCRs that can bind their cognate peptide-MHC and critically, do not transform TCR-antigen binding into a CD3 activation signal. In vitro and in vivo studies confirmed that AED T cells are able to recognize and clear CD19+ tumor cells when co-administered with blinatumomab and yet, in the presence of cognate peptide antigen remain unresponsive, thus lowering the risk of alloreactive responses (e.g., GvHD). These findings may open a new direction for improving the clinical efficacy of biAbs through a combinatorial immunotherapy with allogeneic T cell transfer.

TCRs with a Mutation in the Alpha Connecting Peptide Motif (aCPM) Lose the Ability to Respond to Antigen and Blinatumomab

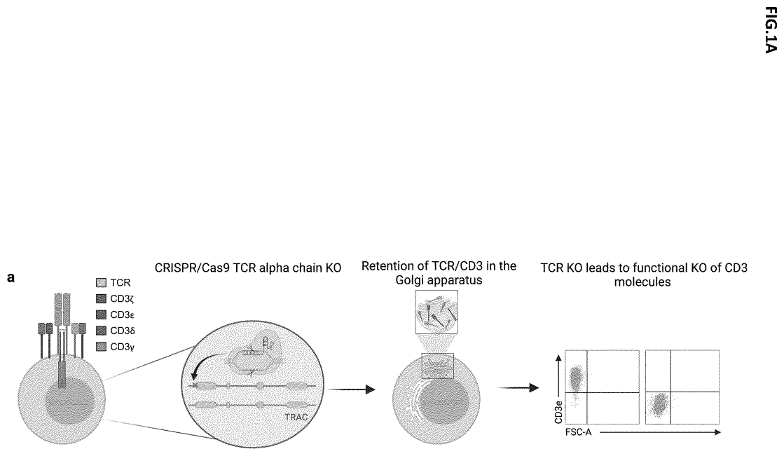

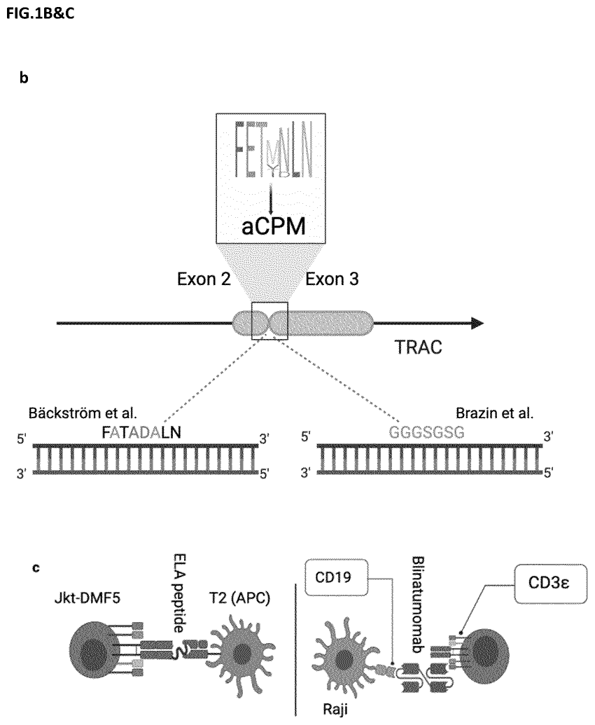

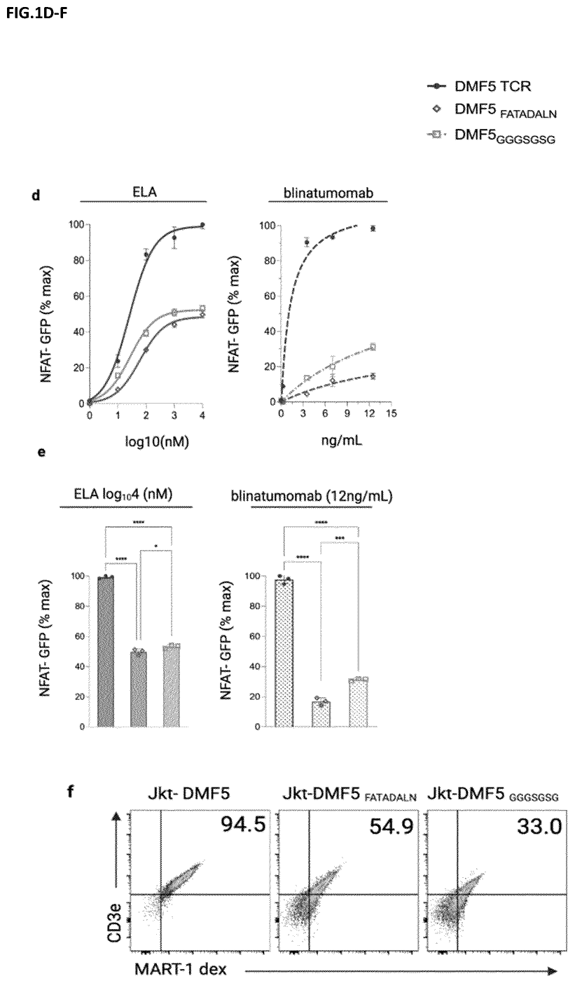

The αβ TCR heterodimer determines T-cell specificity to peptide-MHC (pMHC) complexes. TCR a and B chains consist of recombined variable regions [variable (V), diversity (D) (β chain only) and joining (J) genes], and a constant region made of 3 distinctive segments-a membrane proximal connecting peptide region (CP), a single transmembrane spanning (TM) region and a short cytoplasmic tail lacking signaling domains. CD3 molecules (γε, δε, ζζ), responsible for intracellular signaling and T cell activation are associated to TCRs through charged interactions in the transmembrane regions. These interactions secure accurate assembly of the TCR-CD3 complex within endoplasmic reticulum (EM) and Golgi apparatus ensuring only a functional TCR-CD3 unit is present on the plasma membrane. Disrupting expression of any of the TCR chains (e.g., CRISPR-Cas9-mediated knockout of TCRa) also results in a complete knockout of all CD3 co-receptor subunits and their signaling domains, thus rendering T cells unresponsive to both TCR- and CD3-mediated stimulation (FIG. 1a).

Previous research using mouse T cells and the structural components of TCR signaling revealed that mutations in the sequence motif (FETDxNLN) of the TCRa connecting peptide domain (aCP), drastically reduce (>100-fold) T cell responsiveness to cognate antigen (peptide-MHC), while not disrupting CD3-mediated activation (FIG. 1b) (Brazin et al.; Immunity; 2018; 49; p.829-841.e6 and Bäckström et al.; Immunity; 1996; 5; p.437-447).

The inventors first set out to investigate if these mutations in the aCP of human TCRs could result in a molecular decoupling of TCR and CD3 signaling. As a model cell line, the inventors used a previously engineered human Jurkat T cell line, which has no endogenous TCR and CD3 expression (via Cas9-mediated knockout of TCRa chain) and possesses a nuclear factor of T cell activation (NFAT)-GFP reporter, where GFP is expressed following TCR-CD3 mediated activation. The inventors used CRISPR-Cas9 and homology-directed repair (HDR) to genomically integrate the complete DMF5 TCR gene into the Jurkat cell line and restore TCR-CD3 surface expression (Jkt-DMF5) (FIG. 1h). DMF5 is a well-characterised TCR targeting a tumor-associated melanoma antigen (MART-1) and has been used in clinical trials as anti-melanoma immunotherapy. DMF5 is specific to three MART-1 peptides, including a synthetic derivative peptide [ELAGIGILTV (ELA)] that induces the strongest DMF5 TCR activation. In addition, the inventors also generated Jkt-DMF5 cell lines expressing the previously described mutations in the aCPM (Jkt-DMF5_{FATADALN} and Jkt-DMF5_{GGGSGSG}, SEQ ID NO:27 and 28, respectively) (FIG. 1b), which have shown the potential for decoupling TCR and CD3 signaling in mouse experiments. The inventors performed overnight co-culture experiments with T2 cells pulsed with a range of ELA peptide concentrations (10⁰ to 10⁴ nM). Furthermore, the inventors co-cultured the Jkt-DMF5_{FATADALN} and Jkt-DMF5_{GGGSGSG} cells with the target-expressing Raji (CD19+) cells and a range of blinatumomab concentrations (0-12 ng/ml) to investigate their response upon CD3 stimulation. T-cell activation was quantified by GFP expression via flow cytometry (FIG. 1c,d)

However, the inventors' findings with the human-derived Jurkat T-cells were inconsistent with the data reported in mouse T-cells. Jkt-DMF5_{FATADALN} and Jkt-DMF5_{GGGSGSG} showed only 50% reduction to peptide response (FIG. 1c-e) compared to WT Jkt-DMF5 and a drastic reduction (up to 80%) in response to blinatumomab (FIG. 1c-e). The inventors also observed a significant drop in TCR-CD3 surface expression and in MART1-dextramer binding (FIG. 1f). This is not surprising considering the multiple and complex interactions aCPM has with CD3 subunits as depicted in a complete TCR-CD3 complex assembly. Further modifications or engineering of this motif, which is located at the membrane-proximal nexus of all TCR-CD3 components, would likely render TCRs unresponsive to both peptide and bispecific antibodies (e.g. blinatumomab) activation (FIG. 1g).

Structural and Sequence Analysis Reveals a Novel Conserved TCR Motif with the Potential to Decouple TCR-Antigen Binding from CD3-Signaling

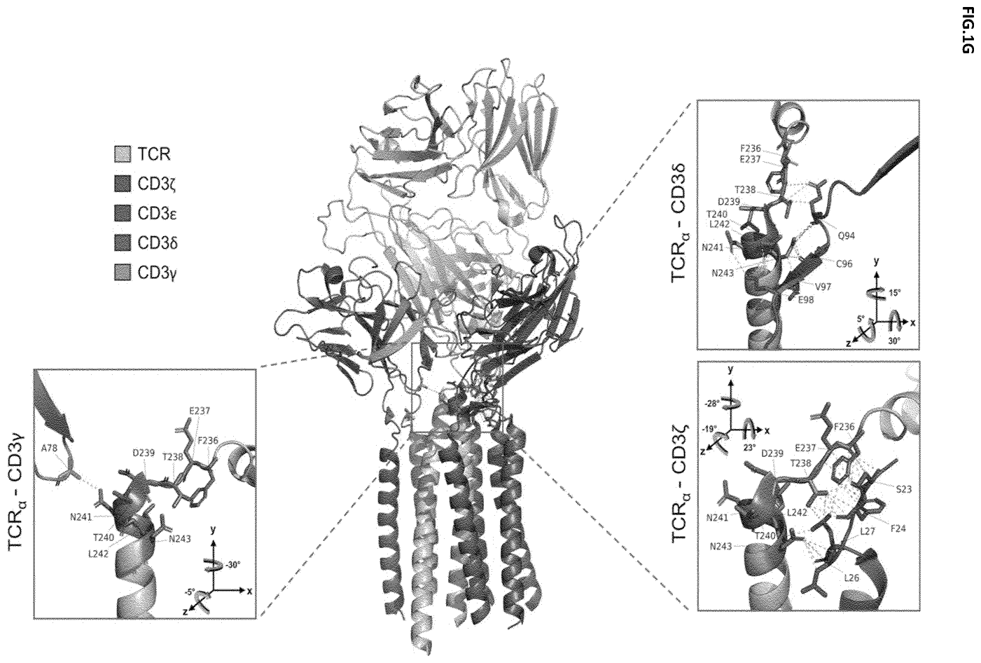

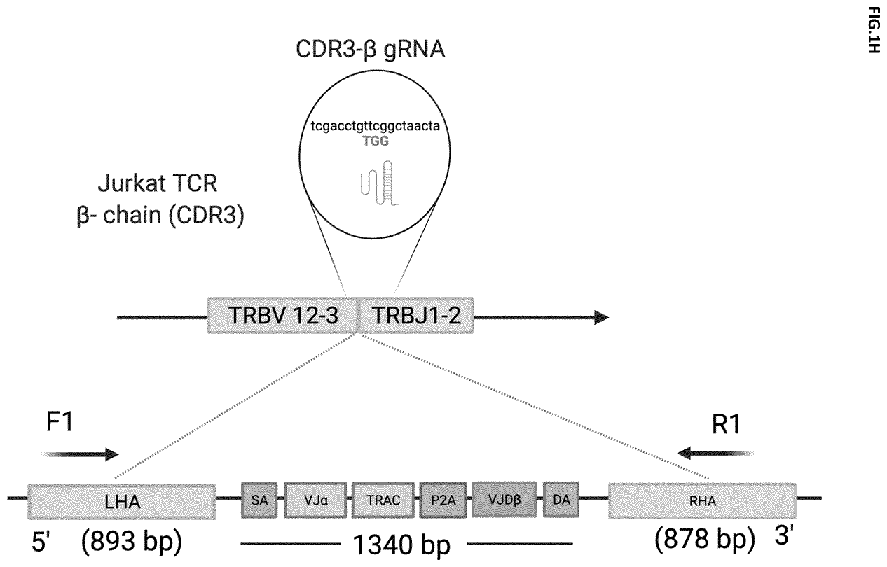

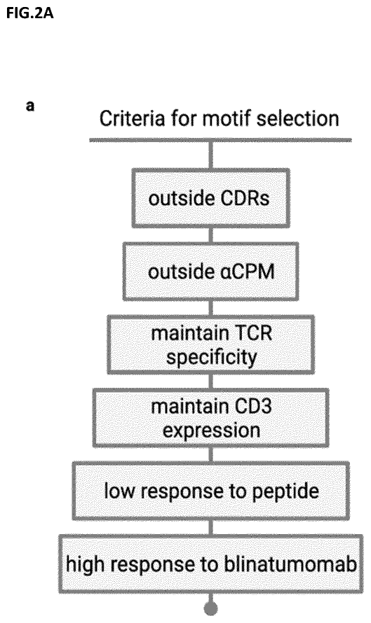

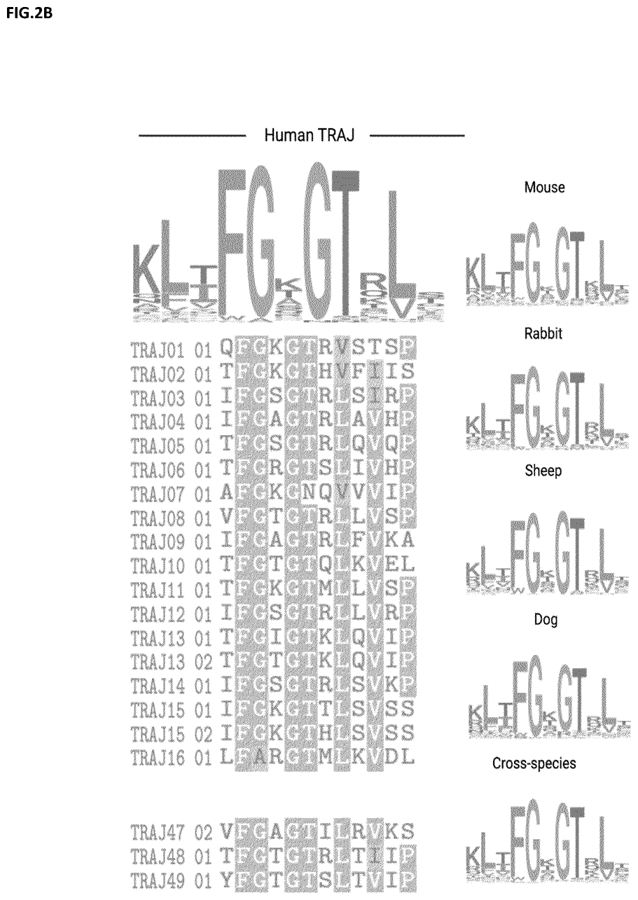

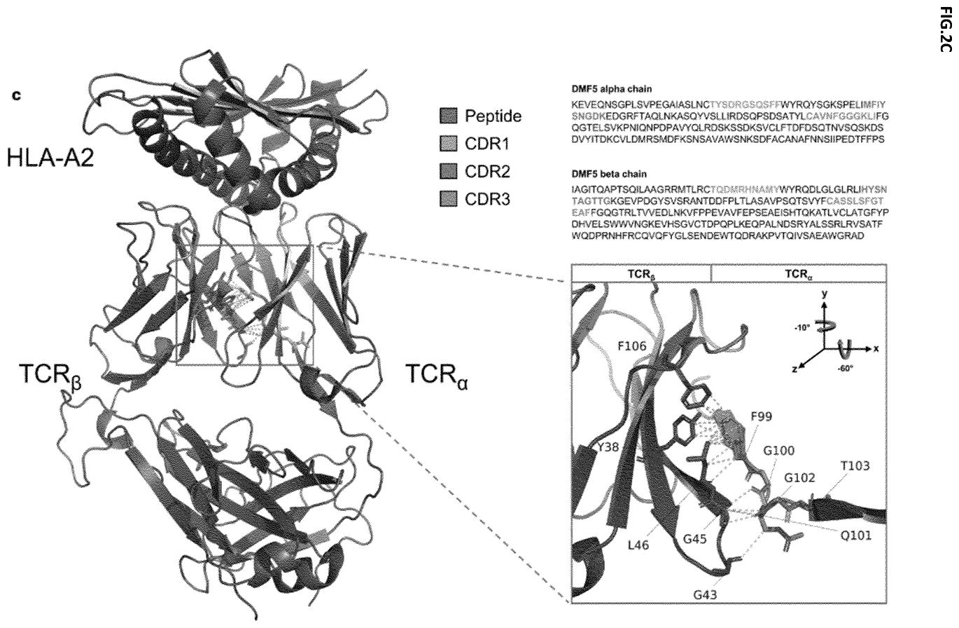

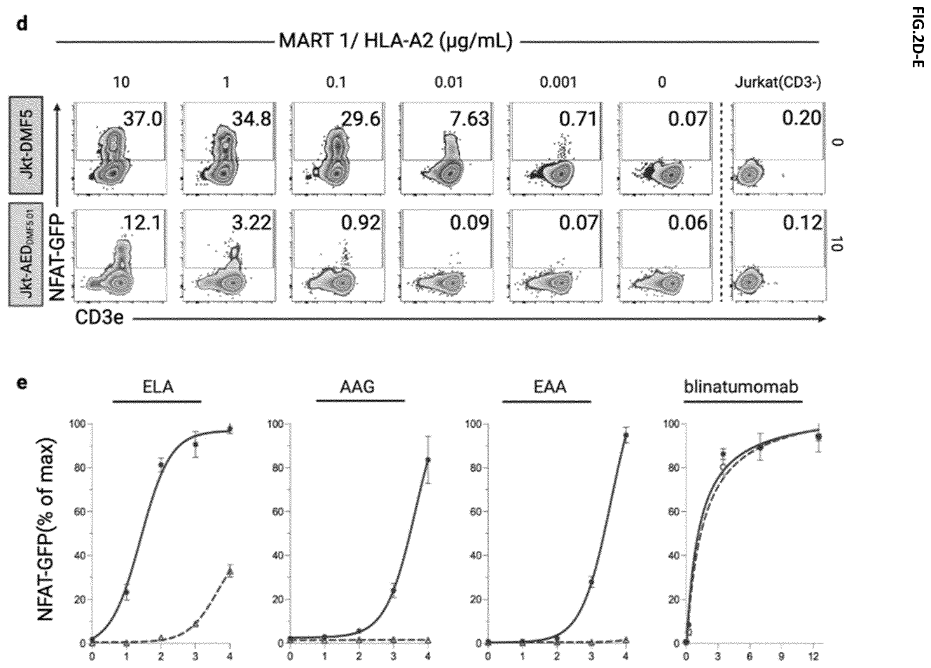

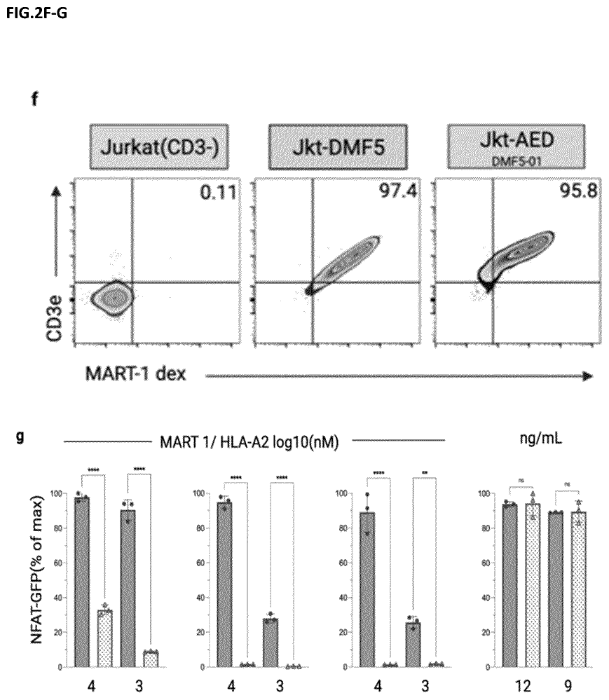

To engineer AED T cells, the inventors first set out to identify potential sequence motifs in TCRs that could be targeted to decouple TCR-antigen binding from CD3 signaling. However, the multi-factorial composition of TCRs renders them susceptible to mutations that can lead to destabilization of the entire TCR-CD3 complex and loss of surface expression. Therefore, the inventors devised a number of criteria based on sequence and functional properties (FIG. 2a). First, such a TCR motif should be highly conserved across TCR germline genes. Second, it should be outside of complementarity determining regions (CDR) since they play a key role in determining molecular specificity to cognate pMHC complexes, and thus, loss of this specificity would not reveal if signaling to CD3 was truly decoupled or if a new and unknown antigen specificity had been introduced. Additionally, the motif should be outside of the aCPM, as the inventors determined that mutations in this region result in abrogation of both TCR and CD3 signaling in human cells (FIG. 1). Another essential criterion is that TCR and CD3 surface expression needs to be maintained. Finally, mutations in this motif must drive a loss of TCR signaling in response to cognate peptide-HLA antigen while maintaining CD3 signaling in response to agonist ligands (e.g., blinatumomab).

To identify candidate motifs meeting such criteria, the inventors performed a multiple sequence alignment of TCR V and J-gene germline sequences within and across species. This led to the identification of the FGxGT motif present in the TCRa J-gene (TRAJ region); this motif is highly conserved in most human germline J-genes and across mammalian species (FIG. 2b). The FGxGT motif is situated just outside the CDR3 region and notably, it occurs not only in the TCRa, but also in the TCRβ chain and in antibody heavy and light chains. Structural analysis revealed that the FGxGT motif forms a number of non-covalent bonds with the TCRβ chain (FIG. 2c). Therefore, the inventors hypothesized that disrupting these contact points might weaken the TCR-αβ association and inhibit signal transduction to CD3 subunits. The inventors used a structure-guided approach to design a candidate AED T cell variant: the mutated motif (FEQWT, SEQ ID NO:9) was incorporated into the backbone of the DMF5 TCR and integrated via Cas9-mediated HDR into the genome of Jurkat cells (Jkt-AED_{DMF5-01}) (FIG. 1h).

One of the important functional criteria required for AED T cells is to maintain TCR binding specificity to cognate peptide-HLA. Labeling of Jkt-AEDDMF5-01 with peptide-HLA dextramers (MART-1-HLA2) revealed a nearly identical binding profile compared to the wild-type Jkt-TCR_{DMF5} (with unmutated FGQGT sequence) (FIG. 2f). Next, the inventors investigated the response of the Jkt-DMF5 and Jkt-AED_{DMF5-01} T cells to MART-1 peptides. In these assays the inventors included naturally occurring peptides [AAGIGILTV (AAG, SEQ ID NO:60) and EAAGIGILTV (EAA, SEQ ID NO:61)] as well as the previously used synthetic peptide (ELA). T cells were co-cultured with T2-peptide-pulsed cells and flow cytometry analysis was performed to examine the GFP response (FIG. 2d). At the highest concentration of ELA peptide (10UM), the Jkt-AED_{DMF5 01} cells showed more than 3-fold reduction in GFP expression. Moreover, it required a peptide concentration of >500-fold higher for Jkt-AEDDMF5-01 to reach the same level of activation as the Jkt-DMF5, and furthermore Jkt-AED_{DMF5 01} cells produced a maximum activation that was 70% lower than Jkt-DMF5 cells (FIG. 2e). A similar pattern was observed across all three MART-1 peptides, with Jkt-AEDDMF5-01 showing virtually no activation when binding the naturally occurring peptides. Importantly, AED_{DMF5 01} T cells co-cultured with Raji tumor cells (CD19+) and blinatumomab were equally responsive as the WT DMF5 TCR across all concentrations tested (FIG. 2 e.g). These results provide evidence that FGxGT motif can be engineered to decouple TCR-antigen binding from CD3-signaling.

### Identification of AED T Cells Across Different TCRs by Mutagenesis and Functional Screening

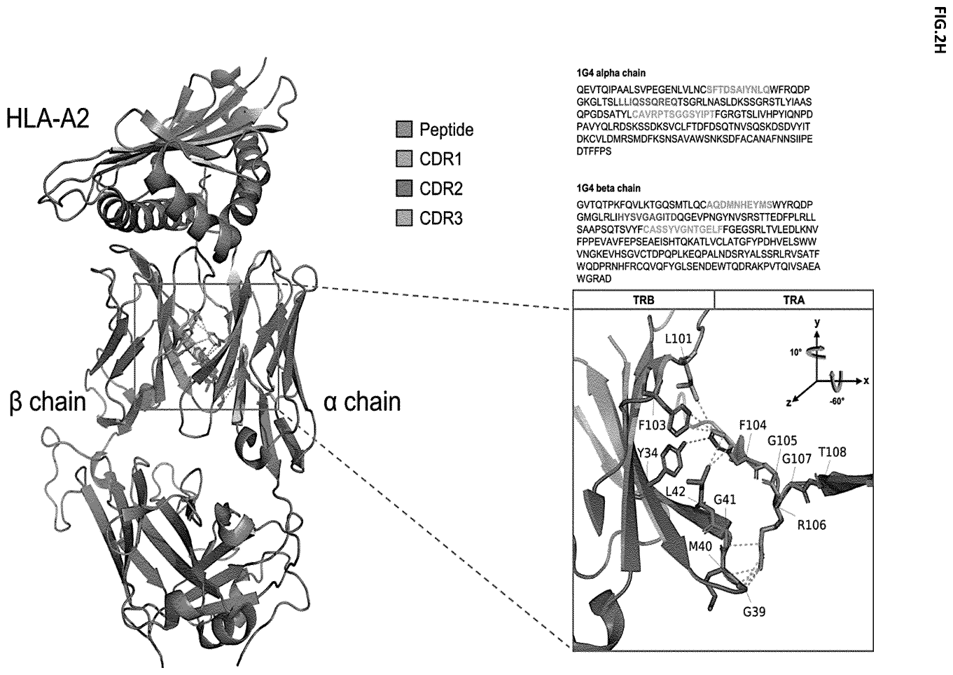

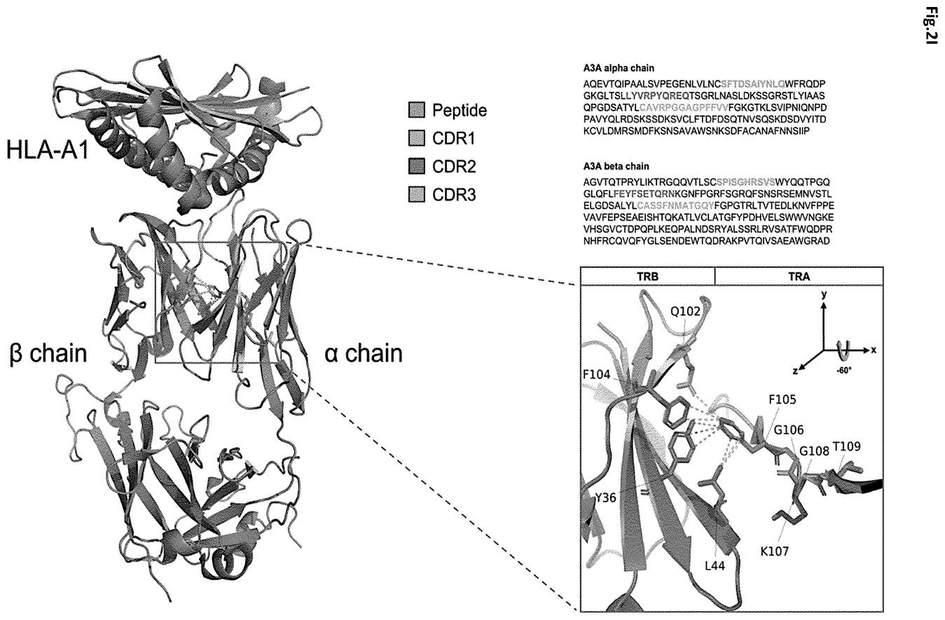

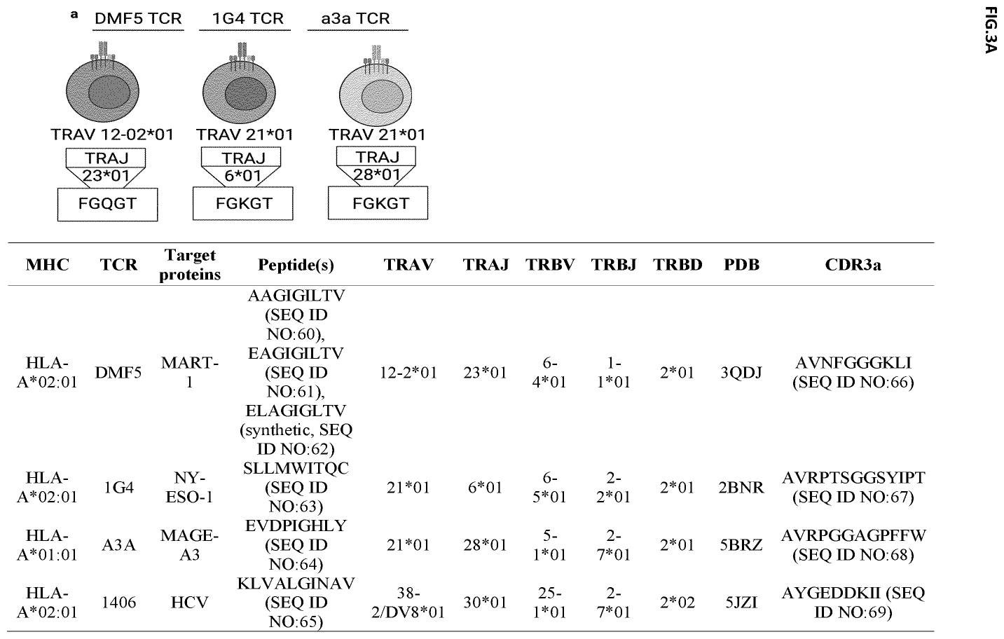

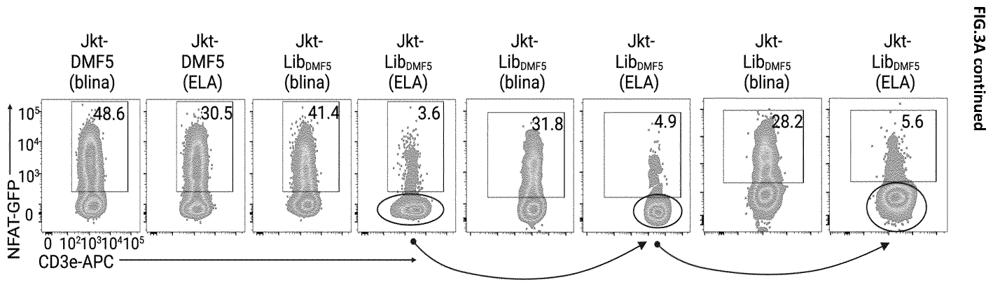

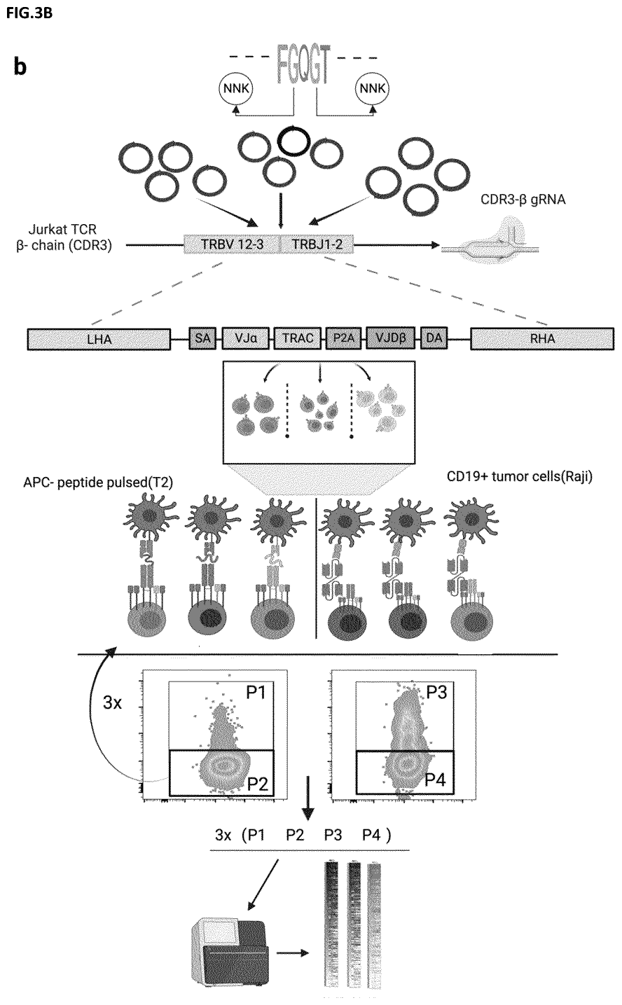

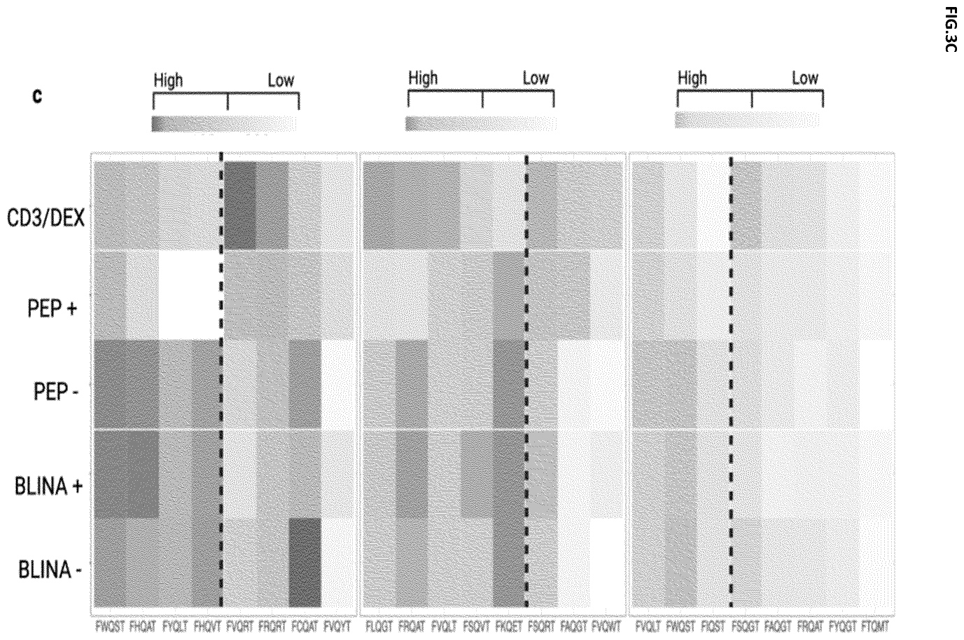

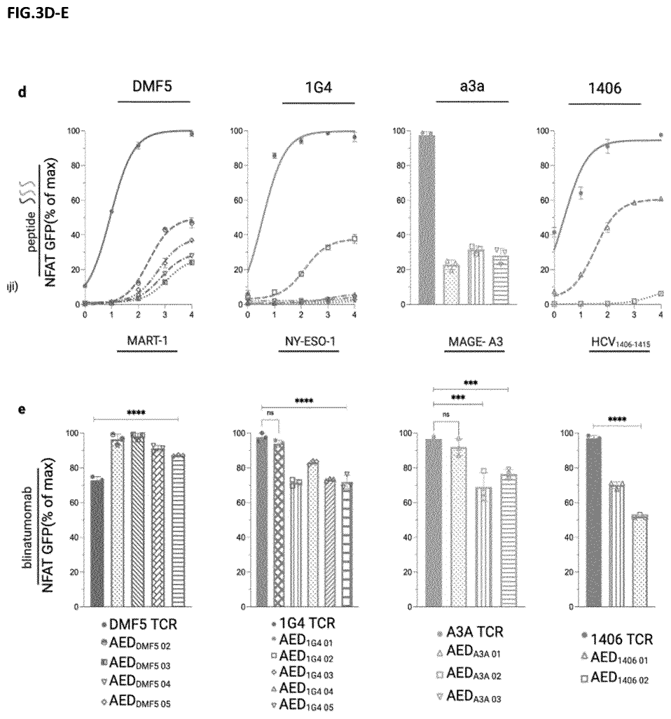

Structural studies of TCRs have shown substantial similarities in the spatial confirmation of TCR α and β-chains. Hence, the inventors initially introduced the same mutations from the AED_{DMF5 01} T cells into two additional TCR clones: TCR 1G4, with specificity to tumor-associated antigen NY-ESO-1 [peptide: SLLMWITQC (SLL, SEQ ID NO:63)]) and a3a TCR, an engineered TCR with a high affinity to melanoma-associated MAGE-A3 antigen [peptide: EVDPIGHLY (EVD, SEQ ID NO: 64)] (FIGS. 2h and i). A3a TCR has caused a lethal cross-reactivity reaction to myocardial titin protein and the inventors wanted to see if it is possible to reverse engineer it to be unresponsive to the EVD peptide in spite of the 4 substitutions in the CDR2 of the α-chain. The three TCRs use different V-gene and J-gene germlines in both a and β-chains. In addition, within the FGxGT motif, the “x” position varies: DMF5 uses glutamine (Q), while 1G4 and a3a use lysine (K), a most common amino acids in this position across human J-genes (FIG. 3a). However, the same mutations in the FGxGT motif in the 1G₄ and a3a TCR either failed to express entirely or show a substantial loss in surface expression and blinatumomab activation.

To further investigate the sequence landscape, the inventors generated mutagenesis libraries of the FGxGT motif on the backbone of clones DMF5, 1G4 and a3a, whereby each TCR had a starting motif of FGQGT. Libraries were designed with both G amino acids being replaced with degenerate codons (NNK), resulting in a theoretical diversity of 400 variants per TCR. The TCR libraries were integrated into Jurkat cells via CRISPR-Cas9 HDR, as previously described (FIG. 1h). Variants were selected based on both CD3 surface expression and cognate peptide-HLA dextramer binding, and in a second step functionally screened by either co-culture with peptide pulsed antigen presenting (T2) cells or co-culture with Raji cells and blinatumomab (FIG. 3b). For DMF5 and 1G4 TCR libraries in Jkt cells, T2 cells were pulsed with ELA and SLL peptides respectively, which are presented on HLA A*20:01. For the a3a library, EJM cells were used since they naturally express the MAGE-A3 antigen and the corresponding EVD peptide on HLA A*01:01 (FIG. 3a). Enrichment and selections were performed by fluorescence-activated cell sorting (FACS) based on the expression of NFAT-GFP (GFP-high and GFP-low populations) (FIG. 3a). Genomic DNA was extracted and targeted amplification of the FGxGT motif from TCR alpha regions by PCR was performed, followed by deep sequencing (Illumina MiSeq). Analysis of deep sequencing data (FIG. 3c) revealed no functional mutational variants conserved across all TCRs, however for each TCR clone the inventors identified several variants with decoupled specificity to peptide-HLA and CD3 activation from blinatumomab (FIG. 3c). To further validate these findings, several of the AED-T cell candidates were challenged with antigen (FIG. 3d) or tumor cells and blinatumomab (FIG. 3e). In addition, the inventors introduced two variants into an alloreactive 1406 TCR. Alloreactive TCRs are responsible for GvHD in allo-transplants as they can express multiple TCRs with different peptide-HLA-specificity. 1406 TCR was discovered when a hepatitis C virus (HCV)-infected HLA-A2-individual received an HLA-A2+ liver allograft. It was subsequently shown to recognize the HCV nonstructural protein 3 (NS3): 1406-1415 epitope with high specificity when presented by HLA-A2. For each TCR clone, the inventors identified mutational variants in the FGxGT motif that displayed a substantial reduction in response to cognate peptide antigen presentation and a varying performance when challenged with blinatumomab that is independent from their peptide response. Notably, several mutational variants of the high affinity a3a TCR could be reduced to 20% of its activity, while maintaining a similar activation profile in response to blinatumomab and CD19+ tumor cells.

Functional Assays Confirm that TCR-Antigen Binding does not Drive Proliferation or Cytokine Secretion from Primary Human AED T Cells

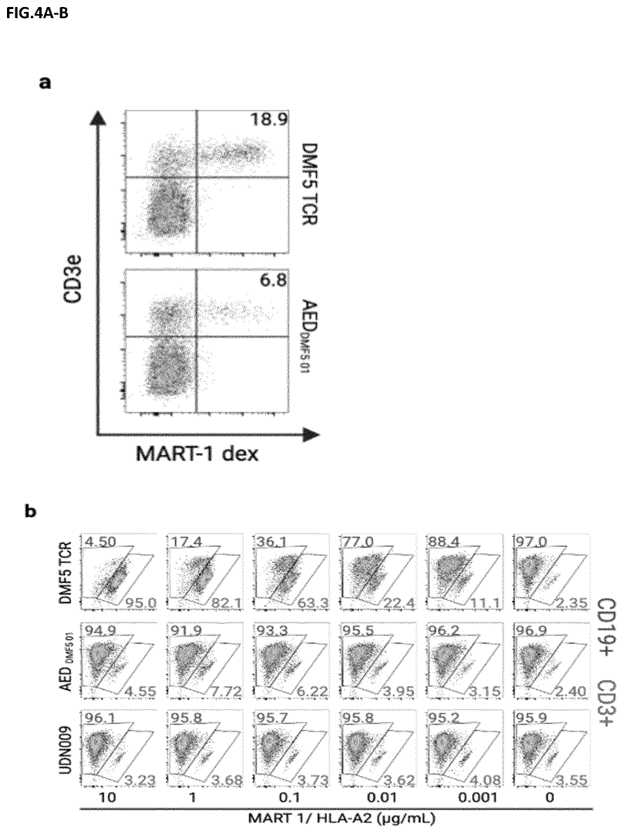

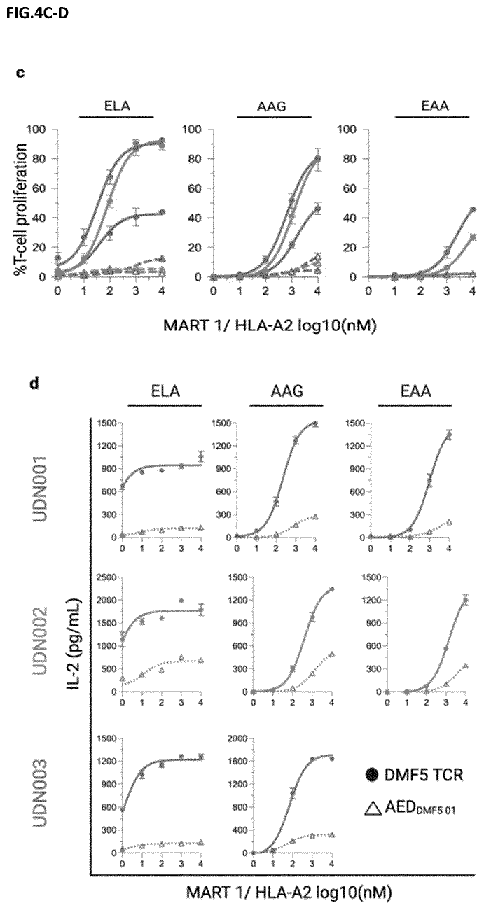

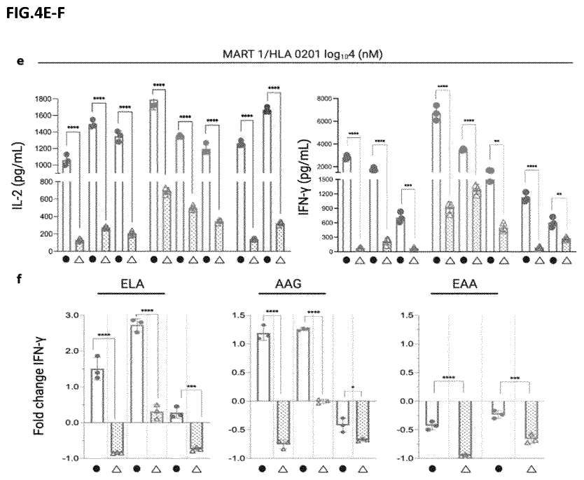

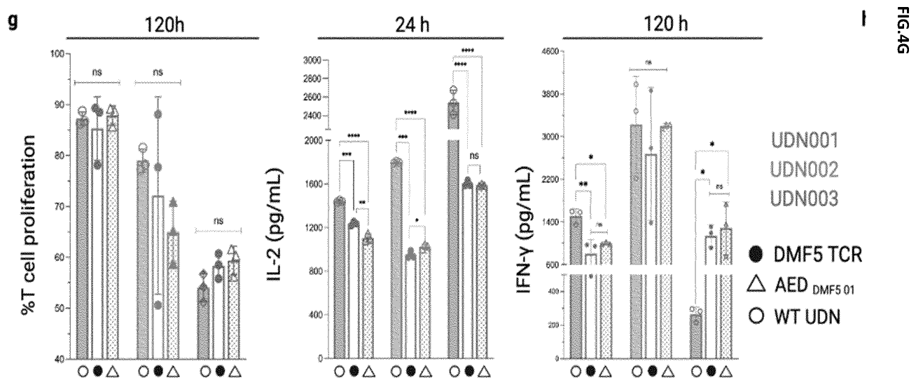

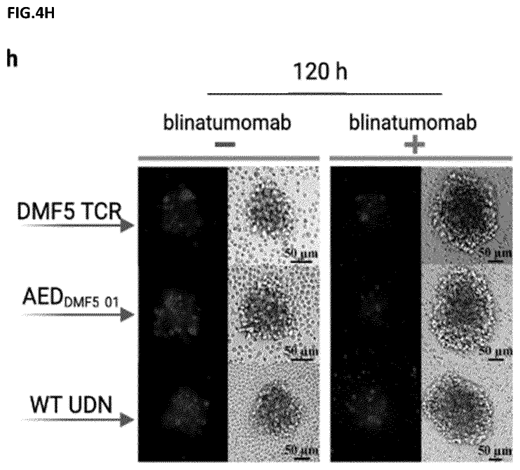

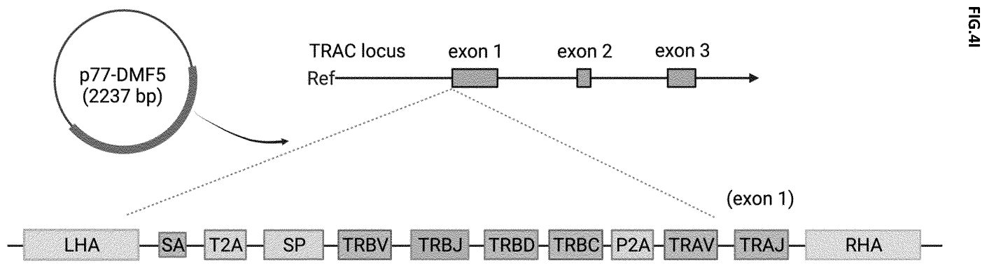

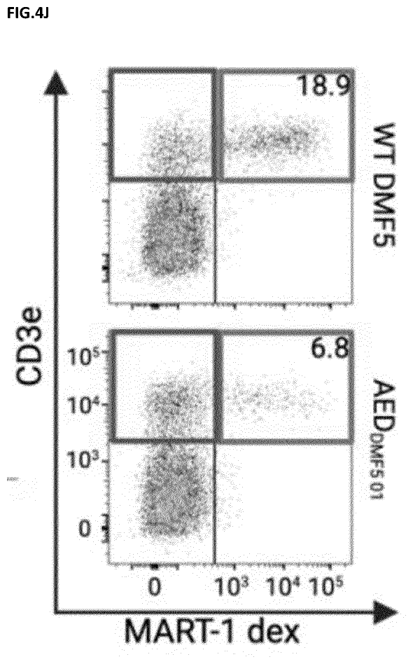

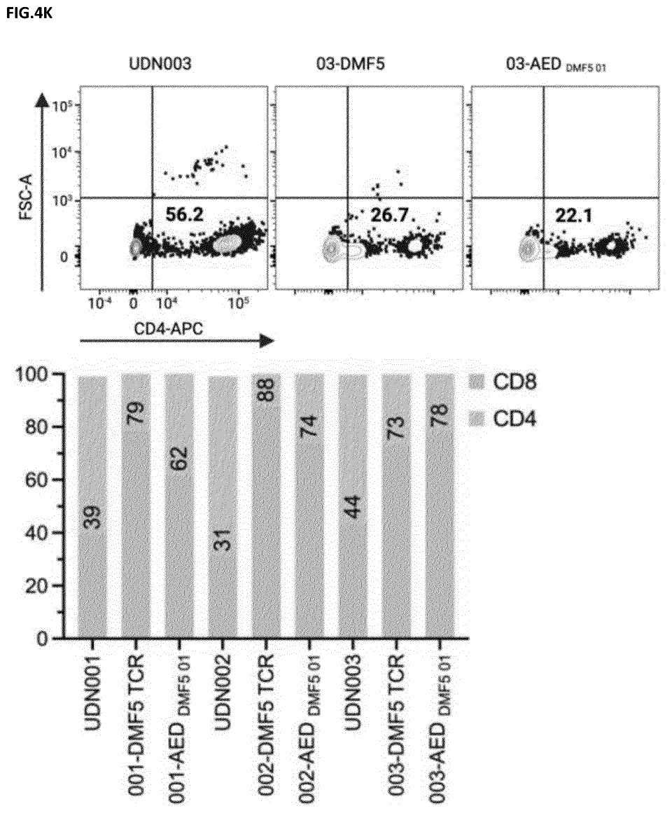

To validate the results of AED T cells in the Jurkat cell line model, the inventors next investigated if similar specificity and functional profiles could be observed in primary human T cells. To this end, the inventors used Cas9-mediated HDR to genomically integrate the AED_{DMF5-01} TCR gene cassette upstream (5′) of the TCR α-chain constant region (TRAC) (FIG. 4i). Following the electroporation of CRISPR/Cas9-ribonucleoproteins (RNP) and dsDNA HDR templates, AED_{DMF5 01} T cells and WT DMF5 T cells were isolated by FACS based on binding to MART-1 dextramer and CD3 surface expression (FIG. 4a). The inventors observed a smaller fraction of MART-1 dextramer and CD3 double positive cells in AED_{DMF5 01} T cells compared to WT DMF5 (FIG. 4j). Sequencing results revealed that the reduction is most likely caused by the mutated alpha chain in AED_{DMF5 01}. The FGxGT motif is at the TCR αβ-chain interface, and amino acid substitutions required for decoupling of the TCR-antigen binding and CD3-signaling also leads to less preferential pairing with the cognate DMF5 β-chain. Similar results were seen in multiple T cell donors independent of HLA configuration. Next, the inventors examined the CD4:CD8 ratio in the T cell populations across three donors and discovered that AED_{DMF5 01} T cells showed a higher ratio of CD8 (1:2) compared to WT donor cells (2:1) (FIG. 4k). A similar ratio was observed in cells engineered with the WT DMF5 TCR. This is unsurprising as DMF5 TCR is naturally associated with the CD8 molecule, and thus both engineered TCRs (DMF5 WT and AED_{DMF5-01}) were more favorably expressed in CD8 T cells.

Next, the inventors evaluated in vitro primary T cell proliferation of AED_{DMF5 01} T cells and WT DMF5 T cells by performing co-cultures with antigen presenting (T2) cells pulsed with MART-1 peptide antigens. In addition to multiple peptide concentrations, T cells were co-cultured with T2-peptide pulsed cells at a 10:1 ratio respectively to better mimic the abundance (avidity) of antigen in healthy tissue. The experiments were conducted over five days and T cell to T2 ratio was analyzed via flow cytometry (FIGS. 4b and c). AED_{DMF5-01} T cells demonstrated minimal proliferation at the highest concentration of peptide (10 μg/mL). In contrast, at the same peptide concentration, DMF5 T-cells completely overtook the T2 cell population, reaching more than 95% of the entire cell population, while in the samples with the non-pulsed T2 cells, WT DMF5 T cells remained at ˜ 2.5%, similar to the other non-peptide pulsed T2 controls.

To examine the dynamics of cytokine production from AED T cells, the inventors evaluated the secretion of interleukin-2 (IL-2), a key regulator of T cell function and proliferation, as well as interferon-gamma (IFN-γ), an essential molecule for cytotoxic activity of CD8 T cells. Assays were performed with three cognate peptides (ELA, AAG and EAA) and T cells derived from three different healthy donors. To differentiate between a truly disabled and only postponed response to peptide, the inventors collected supernatants at 24 h and 120h time points and performed enzyme-linked immunosorbent assays (ELISA). Analyses revealed very low levels of IL-2 produced by AED_{DMF5-01} T cells across all peptides and their various concentrations. Even at the highest peptide concentration (10 μg/mL) AED_{DMF5-01} T cells produce only a minor fraction (˜20%) of IL-2 relative to WT DMF5 T-cells (FIG. 4d,e).

At 120 h, IL-2 was undetectable in AED_{DMF5 01} and WT DMF5 T cells. This is most likely due to its consumption by T cells to drive their proliferation. For IFN-γ at 120 h, secretion levels from AED T cells were significantly lower than WT DMF5 T cells, and notably the strongly activating peptides (ELA and AAG) continued to drive increased IFN-γ production from WT DMF5 T cells through the entire duration of the experiment (FIGS. 4e and f). In contrast, IFN-γ levels decrease significantly over time from AED_{DMF5 01} T cells (FIG. 4f)

Having established that AED T cells derived from primary human T cells were not responsive to TCR activation from cognate peptide antigens, the inventors next aimed to assess their capacity of being activated through the CD3 receptor. Thus, the inventors evaluated the proliferation and cytokine production from AED T cells when co-cultured with the CD19+ tumor cell line (Raji B cells) and in the presence of blinatumomab (FIG. 4b). Across all donors, the inventors observed no significant difference in T cell proliferation following co-cultures (120h) between WT donor, WT DMF5 and AED_{DMF5 01} T cells. However, likely due to the different CD4: CD8 T cell ratio, WT donor T cells produced more IL-2 than both AED_{DMF5-01} and WT DMF5 T cells, while levels of IFN-γ were more similar, or as observed in donor 3 (UDN003), elevated in WT DMF5 and AED_{DMF5-01} T cells. Lastly, the inventors used fluorescence microscopy to capture the interactions between T cells and tumor cells with and without the addition of blinatumomab (FIG. 4h). In all the groups without blinatumomab, T cells were dispersed around tumor cells and did not inhibit tumor growth (red cluster). Similarly, across all groups, the inventors observe that the treatment with blinatumomab activates T cells leading to their proliferation and enables them to infiltrate and encircle the tumor cell cluster, thus limiting their growth. These results show that AED T cells remain fully functional and capable of CD3-activation upon biAb (blinatumomab) engagement.

Primary Human AED T Cells Combined with Blinatumomab in Xenograft Mouse Models Show Potent Anti-Tumor Immunity and Absence of Alloreactivity

Next, the inventors aimed to determine the activity of AED T cells in vivo. The inventors hypothesized that in the presence of blinatumomab AED T cells would be able to clear tumor cells as effectively as conventional T cells (e.g., WT donor T cells). To this end, the inventors used an established human tumor xenograft mouse model, where immunodeficient nod-scid-gamma (NSG) mice were engrafted subcutaneously with luciferase-expressing Raji (CD19+) tumor cells (Raji-RFP-LUC) and two different groups of primary human T cells: WT donor and AED_{DMF5-01} T cells (a control group with Raji only cells was also used). Each T cell population was administered as a 1:1 mix of two different donors. Mice then received intravenous injections (tail vein) of blinatumomab for five consecutive days; control groups with no blinatumomab administration were also included (FIG. 5a). On day 0, each group of mice had a comparable luciferase activity (FIG. 5b) and the experiment was terminated when all the mice in the control group (tumor cells only) reached a terminal bioluminescence signal (>5×10⁹ photons/s).

After seven days post-engraftment and two days after the final blinatumomab dose, all mice receiving blinatumomab treatment showed no sign of tumor progression and no detectable luciferase activity (FIG. 5b,e).

However, unexpectedly, mice receiving the WT donor T cells had similar results as groups receiving blinatumomab (FIG. 5c,e). On the 28th day, four mice from each WT donor group (with and without blinatumomab) were sacrificed to uncover the reasons for this development. However, at this time point, there was no detectable presence of tumor cells or T cells. Given the fact that Raji cells were not engineered to downregulate HLA-I expression, the most likely explanation of the observed tumor rejection in mice with WT donor T cells and no blinatumomab is due to an alloreactive T-cell response, which was previously undetected with in vitro experiments. On the contrary, AED_{DMF5 01} T cells without co-administration of blinatumomab did not induce anti-tumor cell responses (sustained growth of Raji cells in 4 of 5 mice), suggesting that AED_{DMF5 01} T cells do not drive alloreactive responses (FIG. 5d,e). Over the course of the experiment (42 days), mice that received AED_{DMF5 01} T cells and blinatumomab showed undetectable levels of tumor growth, similar to the control WT donor T cells and blinatumomab, and all the mice survived (FIG. 5d-f). In contrast, mice that did not receive blinatumomab gradually reached the endpoint of tumor growth (with the exception of the group receiving WT donor T cells). In order to determine the cellular compositions in mice that did not clear tumors (no blinatumomab treatment), immunofluorescence and chromogenic staining were performed on the tumor only and tumor with AED_{DMF5 01} T cell groups (FIG. 5g). This analysis revealed sparse presence of AED_{DMF5 01} T cells within tumors without characteristic T cell clusters suggesting that no proliferation or non-specific activation against tumor cells occurred. Together, these findings support the safety of allogeneic AED-T cells in adoptive T cell therapy.

### Methods

### Constructs

The sequence of all wild-type TCR clones (DMF5, IG4 and a3a) were ordered as gene fragments (Twist Bioscience). Briefly, each homology-directed repair (HDR) template consisted of homology arms, a P2A sequence, signal peptide and a complete αβTCR separated with a T2A sequence and was cloned into a pUC19 backbone plasmid (Addgene, #50005) via Gibson assembly (NEB, #E2611). Individual AED-constructs from libraries were generated with site directed mutagenesis. These plasmids were used for HDR template amplification (Kapa Hotstart polymerase). dsDNA HDR templates for transfection were column-purified with DNA clean and concentration kit (Zymo Research, #D4013) and concentration was determined by NanoDrop™ 2000c spectrophotometer (Thermo Fisher, #ND-2000) and concentrated to ˜1 μg/μL by Vacuum concentrator (Eppendorf, #5305000703).

### Cell Lines

The Jurkat leukemia E6-1 T cell line was obtained from the American Type Culture Collection (ATCC) (#TIB152). Jurkats were genomically modified into a TnT TCR display platform (Cas9+, CD8+, NFAT-GFP+, FAS-L−, CD3− and CD4−) prior to AED experiments); the T2 hybrid cell line (#ACC598) and the EJM multiple myeloma cell line (#ACC560) were obtained from the German Collection of Cell Culture and microorganisms (DSMZ) and Raji human Burkitt's Lymphoma cell line was obtained from ATCC (#CCL-86); TnT-Jurtkat T cells, T2-cells and Raji cells were cultured in ATCC-modified RPMI-1640 (Thermo Fisher, #A1049101), and EJM cells were cultured in IMDM (Thermo Fisher, #12440053). All media were supplemented with 10% FBS, 50 U/mL penicillin and 50 μg/mL streptomycin. All cell lines were cultured at 37° C., 5% CO₂ in a humidified atmosphere and routinely tested for Mycoplasma contamination. Cells were passaged every 3 days at a ratio 1:5 to keep the cell concentration under 1E6 cells/mL. Detachment of EJM adherent cell lines for passaging was performed using the TrypLE reagent (Thermo Fisher, #12605010).

### CRISPR-Cas9 Genome Editing of Cell Lines

Transfection of Jurkat-derived cell lines (TnT-Cas9+) was performed by electroporation using the 4D-Nucleofector device (Lonza, #AAF-1003X) and the SE cell line kit (Lonza, #V4XC-1024). Prior to transfection, sgRNA complexes were generated by 1:1 mix of 2.5 μL of custom Alt-R crRNA targeting Jkt-TRB CDR3 sequence (TCGACCTGTTCGGCTAACTA, SEQ ID NO:29) (200 UM, IDT) and 2.5 μL of Alt-R tracrRNA (200 μM, IDT, #1072534) following IDT instructions. For the transfection, cells were maintained at a density between 5×10⁵ and 1×10⁶ cells/mL. 1×10⁶ cells were washed twice with room temperature PBS and resuspended in 100 μl of SE buffer together with 1 μg of the HDR template and 5 μl of sgRNA complex. The cell suspension was mixed gently and transferred into a Lonza electroporation cuvette. Cells were electroporated using program CK116 and were immediately topped with 0.5 ml of prewarmed complete media and rested for 10 min before transferring into a 12-well plate with a Lonza transfer pipette. For Jurkat T cells Alt-R HDR enhancer (IDT, #1081073) was added at 30 μM final concentration and removed after 14h by centrifugation. HDR efficiency was assessed by flow cytometry 4 days post transfection.

### Primary Human T Cells Culture and Genome Editing

Peripheral blood mononuclear cells (PBMCs) were isolated from whole blood of healthy human donors (Blutspendezenturm SRK beider Basel, Universitätspital Basel) via Lymphoprep (Stemcell Technologies, #07861), a standard Ficoll based density gradient centrifugation. Human CD4+ and CD8+ T cells were extracted by magnetic negative selection using an EasySep Human Pan T Cell Isolation kit (STEMCELL Technologies, #17951). Primary T cells were cultured in XVivo-15 medium (Lonza, ##: BE02-060F) with 5% fetal bovine serum (FBS) and 50 μM 2-mercaptoethanol with freshly added 200 IU of recombinant human IL-2 (Peprotech, ##200-02), 100 μg/mL Normocin (Invivogen, #ant-nr-1). Throughout the culture period T cells were maintained at 1×10⁶ cells/ml of media. Every 2-3 days, additional media and IL-2 were added, and cells were transferred to larger culture vessels as necessary. On the day of thawing and magnetic selection, T cells were activated with anti-CD3/anti-CD28 Dynabeads (Thermo Fisher, #11456D). Prior to transfection (day 3) beads were magnetically removed. 5 μl of assembled sgRNA (targeting TRAC locus (AGAGTTTGATCCTGGCTCAG, SEQ ID NO:86) molecules were mixed with 1 μL of recombinant SpCas9 (61 μM, IDT, #1081059) and incubated for ˜10 min at RT. Cas9 RNP complex (6 μL) targeting TRAC locus were added to cells (2×10⁶) resuspended in 100 μL of P3 Primary Cell transfection buffer (Lonza, V4XP-3032) and were transfected using the EO115 electroporation program. 600 μL of FBS-free XVivo-15 media was added to the Lonza cuvettes and cells were incubated at 37° C. (30 min). Cells and media were transferred to a 12-well plate and supplemented with IL-2. 2 h later, AAV6-TCRs (Vigene Biosciences) at the MOI of (2×10⁵) were added to the wells and incubated overnight at 37° C. The following day, wells were supplemented with 2.3 mL of complete media (+FBS).

### Flow Cytometry

Samples were acquired on either LSRFortessa (BD Biosciences) or a CytoFLEX (Beckman-Coulter) cytometers and data was analyzed using FlowJo v.10 software. The following antibodies were used in this study. From Biolegend: APC-CD3e (clone UCHT1 #300458) PE-Cy7-CD3e (clone UCHT1, #300420), APC-CD4 (clone RPA-T4, #300552), PE-CD8a (clone HIT8a, #300908), PE-Cy7-CD19 (clone HIB19, #302216), PE-conjugated anti-human TCR α/β (clone IP26, #306707). DAPI viability dye (Thermo Fisher, #62248) was added to antibody cocktails at a final concentration of 1 μg/mL. Cells were washed once in flow cytometry buffer (PBS, 2% FBS, 2 mM EDTA) prior to staining, stained for 20 min on ice and washed twice in flow cytometry buffer before analysis. In co-culture experiments before additional staining reagents, Fc receptors on T2 cells were blocked with TruStain FcX reagent (BioLegend, #422301). Staining with peptide-MHC dextramers was performed for 10 min at room temperature (RT), followed by addition of surface staining antibodies and incubation for 20 min on ice. The following peptide-MHC dextramers were commercially obtained from Immudex: NY-ESO-1₁₅₇₋₁₆₅ (SLLMWITQC (SEQ ID NO:63), HLA-A*0201, #WB2696-PE); MART-1_{26-35(27L)} (ELAGIGILTV (SEQ ID NO:62), HLA-A*0201, #WB2162-PE); MAGE-A3₁₆₈₋₁₇₆ (EVDPIGHLY (SEQ ID NO:64), HLA-A*0101, #WA3249-PE). Peptide-MHC dextramers were used at a 3.2 nM final concentration (e.g. 1:10 dilution) for staining. Cell sorting (FACS) was performed using BD FACSAria III or BD FACSAria Fusion instruments. All the samples were sorted in bulk and used as such to avoid variations in signal and cell behavior arising from single cell variability.

### Peptides and Peptide Pulse

Peptides were generated by custom peptide synthesis (Genscript), re-suspended at 10 mg/ml in DMSO and placed at −80° C. for prolonged storage. For peptide pulsing, T2 cells were harvested and washed twice in serum-free RPMI 1640 (SF-RPMI). Peptides were diluted to 10 μg/mL in SF-RPMI (or to concentrations indicated in figure legends) and the solution was used to make 10-fold dilutions in which cells were resuspended at a concentration 1×10⁶ cells/mL. Cells were incubated for 120 min at 37° C., 5% CO2, washed twice with SF-RPMI, resuspended in complete media and added to co-culture wells.

### APC- and Jurkat T Cell Co-Culture Assays

T2 cells or EJM were used as antigen presenting cells (APCs) in co-culture experiments with either Jurkat WT TCRs or AED-modified TCRs. T cells were at ˜1×10⁶ cells/mL when harvested, pelleted by centrifugation and re-suspended in fresh complete media at 1×10⁶ cells/mL and counted. If not stated otherwise, 1×10⁵ T cells (100 μL) were seeded in 96-well plate (V bottom) wells. Antigen-expressing cells (EJM) or peptide-pulsed cells (T2) were adjusted to 1×10⁶ cells/mL in complete media and 5×10⁴ cells (50 μL) added to each well with an APC: T cell ratio (1:2). Anti-human CD28 antibody (clone CD28.2, #302933; BioLegend) was added as a co-stimulatory signal at a final concentration of 1 μg/mL to all samples (including negative controls). Plates were incubated overnight at 37° C., 5% CO₂. The next day, expression of NFAT-GFP in modified Jurkat T cells was assessed by flow cytometry.

### Co-Culture of Primary T Cells and ELISAs

WT and AED TCR-reconstituted primary T cells were FACS sorted, supplemented with IL-2 and rested for 3 days before the co-culture experiment. After resting, T cells were washed, counted and resuspended in complete primary T cell media. T cells and T2 cells were mixed at a 1:10 ratio (5×10³ and 5×10⁴ cells) in a total of 150 μL of media and incubated overnight at 37° C., 5% CO2. Next day, cells were spun down and supernatant was collected for ELISA experiments. Cells were resuspended in fresh media and cultured for an additional 4 days. Afterwards, supernatant was collected again and cells were assessed by flow cytometry. Concentration of human IL-2 and IFN-γ cytokines were quantified using standard kits (Thermo Fisher, #88-7025-88 and #88-7316-88). Supernatants were diluted in media to fall within the standard curve for the assay. Negative control values were subtracted from each sample point and the concentration was calculated from the standard curves. Measured concentrations of cytokine were plotted versus the peptide concentration and fitted to a 4-parameter logistic model.

### TCR Library Design, Selection and Sequencing

Deep mutational scanning (DMS) combinatorial libraries of the TRAJ motif (FGxGT) libraries for TCR_{DMF5}, TCR_{IG4} and TCRₐ₃ₐ were generated by plasmid nicking mutagenesis as previously described. Briefly, the protocol relies on the presence of a single BbvCI restriction site for sequential targeting with Nt.BbvCI and Nb.BbvCI nickases, digestion of wild-type plasmid and plasmid re-synthesis using mutagenic oligonucleotides. Mutagenic oligonucleotides were designed using the QuikChange Primer Design online tool (Agilent). After nicking mutagenesis, mutated plasmids were transformed into 100 μL of chemically-competent E. coli DH5α cells (NEB, #C2987H) and plated on ampicillin (100 μg/mL) LB agar in Nunc BioAssay dishes (Sigma-Aldrich, #D4803). Serial dilutions of transformed cells were plated separately to quantify bacterial transformants. Plasmid libraries were purified from bacterial transformants using the ZymoPURE Plasmid miniprep Kit (Zymo Research, #D412). HDR templates were amplified from plasmid libraries by PCR and column-purified prior to transfection.

DMS library HDR templates and CDR3B gRNA were used to transfect 1×10⁶ lab modified Jurkat T cells. Firstly, cells were stained with (dextramer) and FACS sorted for the CD3 surface expression. In the second round, cells were challenged separately with their cognate peptide (0.1 μg/mL)/EJM cells and blinatumomab (12 ng/mL) and both GFP− and GFP+ fractions were sorted (SEL1). GFP− peptide fraction was used as the starting population for following selections (SEL 2 and 3). Genomic DNA from all sorted populations was extracted via PureLink Genomic DNA Mini Kit (ThermoFisher, #K182002). Regions of interest were PCR amplified with added TruSeq adapters for 300-PE v3 (600 cycles). MiSeq sequencing was performed in the Genomics Facility Basel.

### In Vitro Microscopy

Raji (5×10⁴) and T (1×10⁴) cells were plated in a 96 half-area well plate (Corning, #CLS3690) with a transparent glass bottom for higher sensitivity. Cells were plated in the X-Vivo media without phenol red (Lonza, #04-744Q) and supplemented with FBS. The well plate was placed in an environmental chamber, which provided a 5% CO2 atmosphere and a humidity of at least 70%. The imaging of the cells was conducted on a fully automated Nikon Ti2 microscope with a 10× magnification (Plan Apo λ10×). Every well containing cells was imaged fully by stitching 3×3 images together with an overlap of 15%. The cells were imaged at 24 h and 96 h. Raji cells were stained prior to the start of the experiment with the CellTracker™ Deep Red (ThermoFisher, #C34565) following manufacturer's protocol. Stained Raji cells were visualized using the mCherry filter cube from Nikon and an exposure time of 50 ms with a light power of 33%.

### Data Analysis and Visualizations

Data analysis was performed using R (version 4.0.1.). Visualizations were generated using the R packages ggplot2 (version 3.3.3) and ggseqlogo (version 0.1, Sequence logo plots). TCR structures were prepared using PyMOL and complete figures and graphics were generated using BioRender software.

### Multiple Sequence Alignment (MSA)

Germline gene sequences for TRAJ, TRAV, TRAC, TRBJ, TRBV and TRBC were obtained for various species from IMGT. MSA was performed for each of these regions within and across species using R-package msa (version 1.20.1, method=“ClustalW”) in R (version 4.0.1).

### Sequencing Analysis

Raw sequencing data from screening libraries were preprocessed and aligned using the MiXCR software package. Data was cleaned to only contain sequences that showed variation in the positions targeted for mutation. Frequency and rank of unique variants was calculated from clone count. Sequences of interest were identified by a decrease in rank and de-enrichment (based on frequency) in the peptide positive fraction, and maintenance of rank and absence of de-enrichment in the blinatumomab positive population.

### Mouse Strains and Study Approval

NOD/SCID/IL-2Rγ-null (NSG) mice were purchased from Charles River Laboratories. Mice were maintained and bred in the EPFL animal facilities in a pathogen-free environment. All animal experimentations were performed in accordance with the Swiss Federal Veterinary Office guidelines and as authorized by the Cantonal Veterinary Office (animal license). Both female and male littermates (aged 5 weeks) were used in the experiments.

### In Vivo Mouse CD19+ Xenograft Tumor Model

Mice were inoculated in the flank with a mixture of 1×10⁵ Raji-RFP-LUC cells and 1.5×10⁶ WT or AED_{DMF5 01} T-cells. Control group (without T cells) received only 1×10⁵ Raji-RFP-LUC cells. Prior the inoculation, cells were washed and resuspended in 100 μl PBS. Before blinatumomab treatment, mice were divided into groups of 5 mice each (Control group had 3 mice), with equal tumour size distribution based on bioluminescent imaging. Blinatumomab (0.1 mg/mouse/injection) was administered through tail-vein injection every day over the course of five days following tumor engraftment. Mice's health and weight were monitored three times per week using body and health performance score sheets.

### Bioluminescence Imaging

Tumor growth was monitored by Bioluminescent imaging (BLI). BLI was performed using the Xenogen IVIS Lumina II imaging system. Briefly, mice were injected i.p. with D-luciferin (150 mg/kg stock, 100 μL of D-luciferin per 10 g of mouse body weight) resuspended in PBS and imaged under isoflurane anesthesia after 5-10 min. A pseudocolor image representing light intensity was generated using Living Image v.4.5 software (Caliper Life Sciences). Mice were sacrificed when bioluminescence intensity exceeded 5×10⁹ photons/second.

### Lentiviral Transduction of Raji_RFP_LUC Cells

For lentiviral transduction of Luciferase (LUC)-RFP vectors, 293T cells were seeded at 30% confluence in 10 cm dishes in DMEM 10% FBS, and transfected the next day with the backbone of interest and the packaging plasmids pMD2.G and d8.9 using FuGENE HD (Promega). Media was changed 16 hours after transfection. The viral supernatant was collected 24 and 48 hours post-transfection and incubated at 4° C. overnight with PEG800. It was then centrifuged at 3500 rpm for 1 hour at 4° C. The pellet was used to infect 200 000 Raji cells in presence of 8 μg/μl polybrene. Transduced Raji-RFP-LUC cells were sorted and maintained in RPMI 1640 with 10% FBS, and 1% penicillin/streptomycin. Cells were then characterized in vitro for Luciferase expression levels and CD19 expression by flow cytometry.

### Immunofluorescence and Immunohistochemistry Stainings

Immunohistochemical detection of CD19 was performed manually. After dewaxing and rehydration, sections were incubated for 10 min in 3% H2O2 in PBS to inhibit endogenous peroxidase. They were pretreated with 10 mM Tri Na citrate pH6 for 20 min at 95° C. using PT module (Thermo Fisher Scientific). Slides were then blocked in 1% BSA in PBS for 30 min. Rabbit anti-human CD19 (rat anti-CD19, clone 60MP31, eBioscience, cat #14-0194-82) diluted 1:500 in 1% BSA was incubated overnight at 4° C. with agitation. After 3 washes in cold PBS, the secondary antibody rabbit (Thermo Fisher Scientific, cat #A-1107) diluted 1:1000 in 1% BSA was applied for 30 min at room temperature. Sections were counterstained with DAPI and permanently mounted.

Analysis of CD3 (rabbit anti-CD3e, clone Sp7, Thermo Fisher, cat #MA5-14524, diluted 1:100) was performed using the fully automated Ventana Discovery ULTRA (Roche Diagnostics, Rotkreuz, Switzerland). All steps were performed on the machine with Ventana solutions. Briefly, dewaxed and rehydrated paraffin sections were pretreated with heat using standard condition (40 minutes) CC1 solution. The samples were incubated with the primary antibody for 1 hour at 37° C. After incubation with rabbit Immpress HRP (Ready to use, Vector laboratories Laboratories), chromogenic revelation was performed with ChromoMap DAB kit (Roche Diagnostics, Rotkreuz, Switzerland). Sections were counterstained with Harris hematoxylin and permanently mounted. Slides were acquired with Leica DM5500 Upright Microscope and analyzed using QuPath (Protocol designed and performed by the EPFL Histology Core Facility).

### Statistical Analysis

Statistical significance involving two groups were determined by two-tailed, unpaired Student's t-test. For comparison among 3 groups or more, analysis of variance (ANOVA) with multiple comparisons was used, and the P value was adjusted with Tukey's or Sidak's correction. Statistical significance in the Kaplan Meier curve was determined using the Mantel-Cox log rank test. All P values were calculated using the GraphPad Prism software (v.9.1.2). In all graphs, error bars represent s.d.

### Example 2: Activation of AED-Modified Jurkat T Cells by Melanoma Cell Lines

WT DMF5 or AED-modified Jurkat T-cells were at ˜1×10⁶ cells/mL when harvested, pelleted by centrifugation and re-suspended in fresh complete media. 1×10⁵ T cells (100 μL) were seeded in 96-well plate (V bottom) wells. 5×10⁴ melanoma cells was added in each well. Anti-human CD28 antibody (clone CD28.2, 566 #302933; BioLegend) was added as a co-stimulatory signal at a final concentration of 1 μg/mL to all samples (including negative controls). Plates were incubated overnight at 37° C., 5% CO₂. The next day, surface expression of CD69 in modified Jurkat T cells was assessed by flow cytometry.

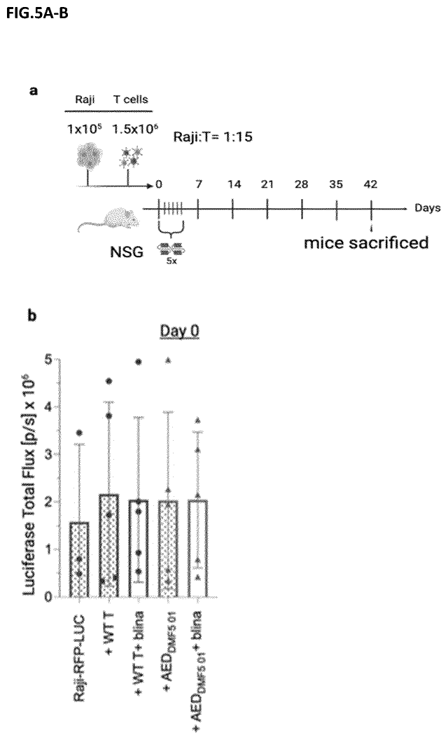

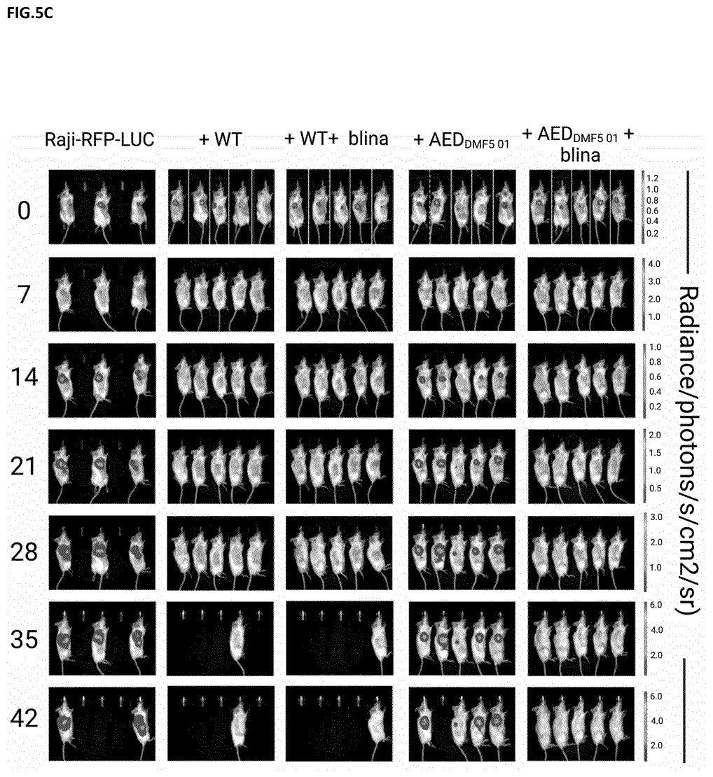

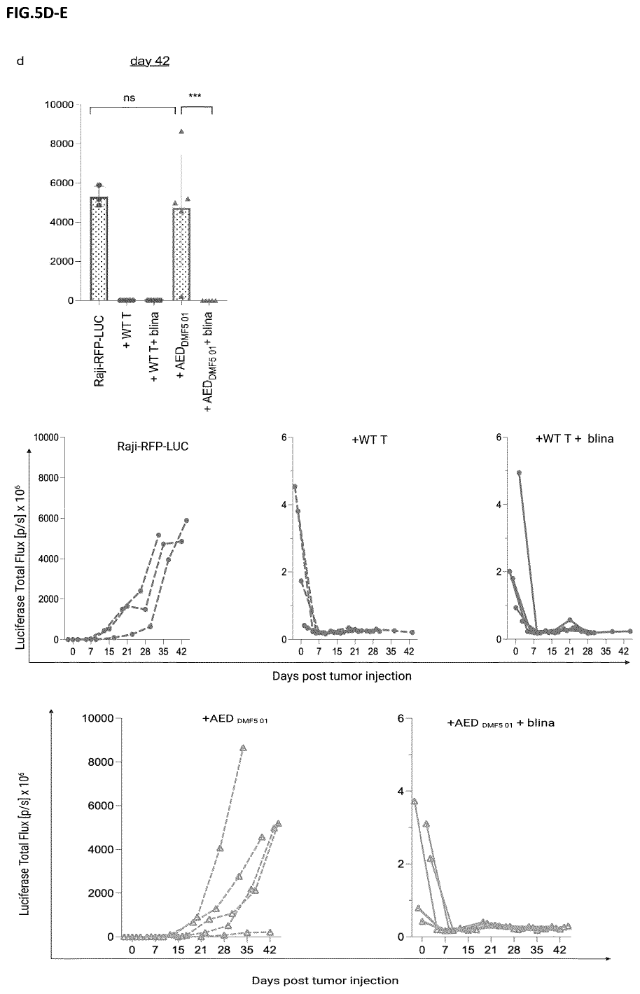

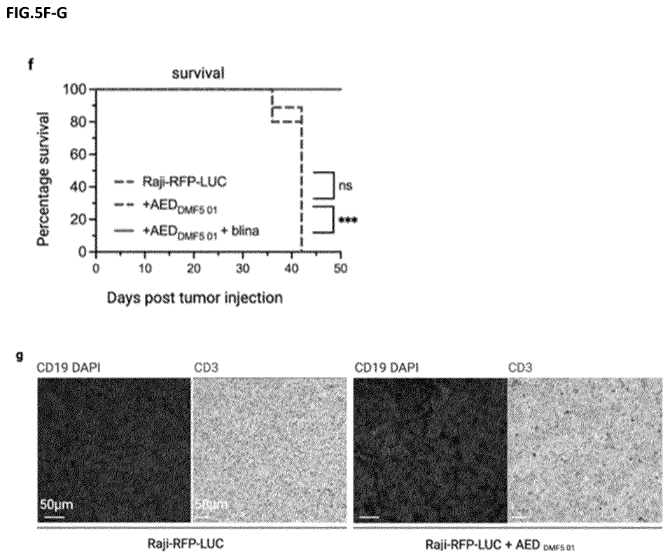

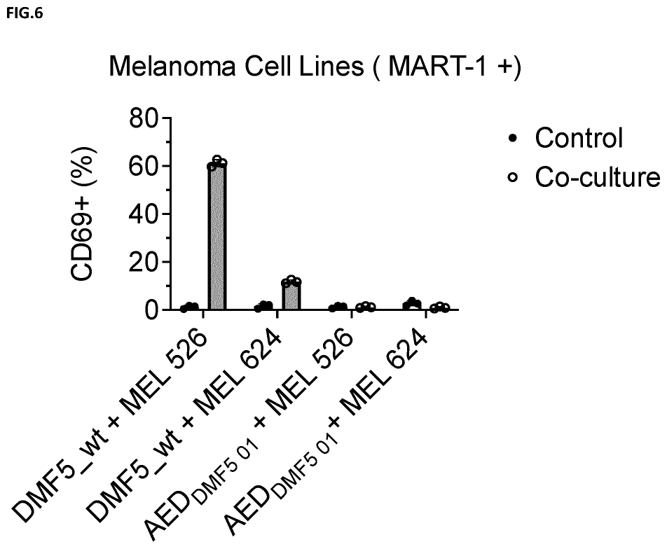

AED_{DMF5 01} Jurkat-T cells were not activated by Melanoma cell lines (Mel526 and Mel624) that naturally express high levels of MART-1 antigen (FIG. 6).

### Example 3: Granzyme B Expression in TCR-Reconstituted Primary T Cells

WT and AED TCR-reconstituted primary T cells were FACS sorted, supplemented with IL-2 and rested for 3 days before the co-culture experiment. After resting, T cells were washed, counted and resuspended in complete primary T cell media. T cells and T2 cells were mixed at a 1:10 ratio (5×10³ and 5×10⁴ cells) in a total of 150 μL of media and incubated overnight at 37° C., 5% CO₂. 4 days after, supernatant was collected, and cells were assessed by flow cytometry. Concentration of human Granzyme B was quantified using standard kits (Invitrogen, #BMS2027-2). Supernatants were diluted in media to fall within the standard curve for the assay. Negative control values were subtracted from each sample point and the concentration was calculated from the standard curves. Measured concentrations of cytokine were plotted versus the peptide concentration and fitted to a 4-parameter logistic model.

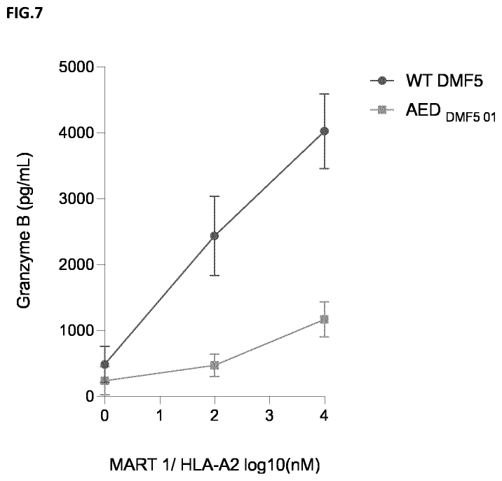

Primary AED_{DMF5 01} T cells produced significantly less Granzyme B than WT DMF5 T cells across extensive peptide concentration and different donors (n=4) (FIG. 7).

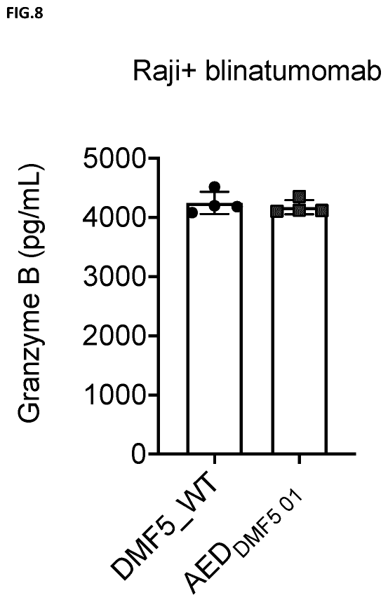

Primary AED_{DMF5 01} T cells were equally responsive in secreting Granzyme-B to blinatumomab stimulation as WT DMF5 T cells. Data represents different unrelated donors (n=4) (FIG. 8).

### Example 4: LAMP-1 Expression in TCR-Reconstituted Primary T Cells

WT DMF5 and AED TCR-reconstituted primary T cells were FACS sorted, supplemented with IL-2 and rested for 3 days before the co-culture experiment. After resting, T cells were washed, counted and resuspended in complete primary T cell media. T cells and T2 cells (pulsed with 10 μg/mL of ELAGIGLTV peptide) were mixed at a 1:1 ratio (1×10⁵ and 1×10⁵ cells) in a total of 150 μL of media and incubated for 4 h with anti-LAMP-1 antibody (#328626) at 37° C., 5% CO2. Cells were washed, stained and analyzed via flow cytometry (Cytoflex).

LAMP-1 expression on T cell surface was significantly lower in Primary AED_{DMF5 01} T cells than WT DMF5 across extensive peptide concentrations and multiple donors (n=2) (FIG. 9).

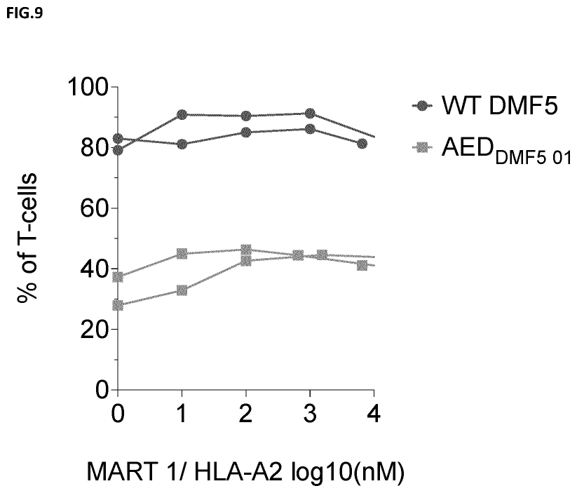

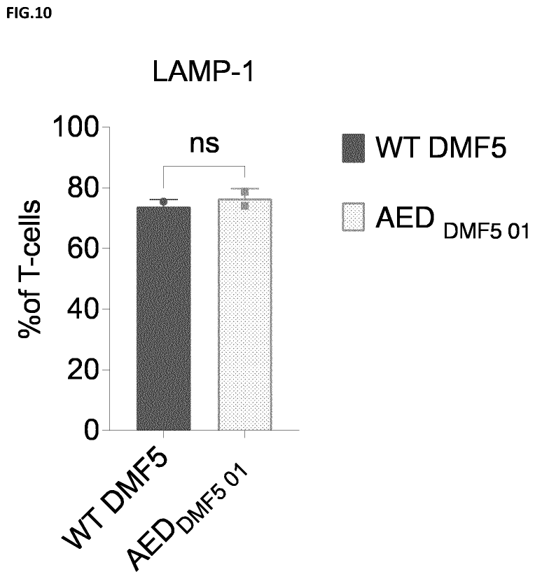

LAMP-1 expression with blinatumomab stimulation: No significant difference in LAMP-1 expression between AED_{DMF5 01} and WT DMF5 across multiple donors (n=2) (FIG. 10).

### Example 5: Stimulation of AED-Modified Jurkat T Cells with Other Bispecific Antibodies

Karpas-422 and WSU-DLBCL-2 cell cells were used as CD20 antigen presenting cells (APCs) in co-culture experiments with either Jurkat WT DMF5 TCRs or AED-modified TCRs. Cells were tested with clinically relevant anti-CD20 antibodies (mosunetuzumab, epocirtamab and glofitamab, recombinantly expressed by SinoBiological Inc). T cells were at ˜1×10⁶ cells/mL when harvested, pelleted by centrifugation and re-suspended in fresh complete media at 1×10⁶ cells/mL and counted. If not stated otherwise, 1×10⁵ T cells (100 μL) were seeded in 96-well plate (V bottom) wells. were adjusted to 1×10⁶ cells/mL in complete media and 5×10⁴ cells (50 μL) added to each well with an APC:T cell ratio (1:2). Anti-human CD28 antibody (clone CD28.2, #302933; BioLegend) was added as a co-stimulatory signal at a final concentration of 1 μg/mL to all samples (including negative controls). Plates were incubated overnight at 37° C., 5% CO₂. The next day, expression of CD69 (Biolegend, #310912) in modified Jurkat T cells was assessed by flow cytometry.

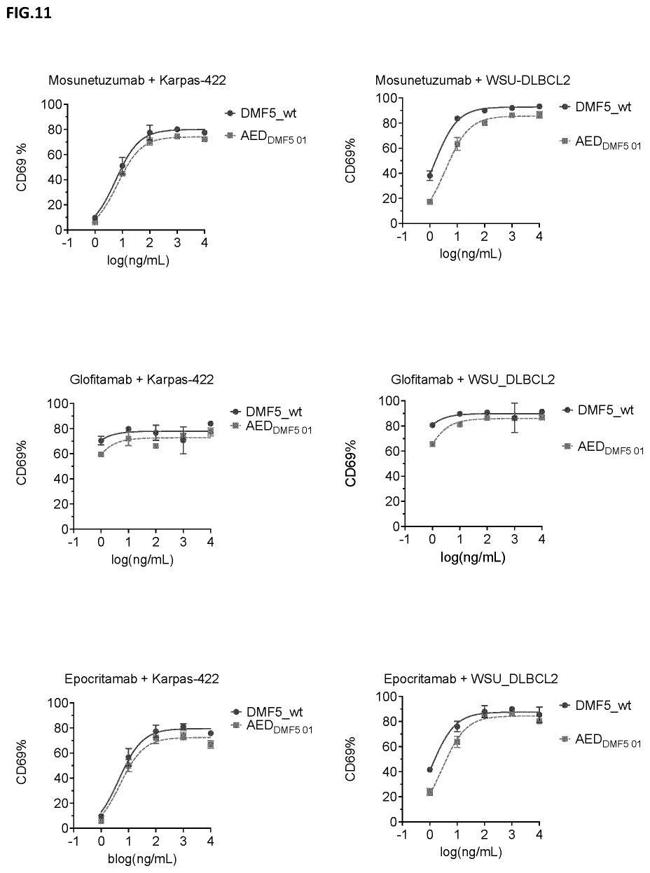

AED_{DMF5 01} and WT DMF5 Jurkat T cell stimulation with multiple biAb and two CD20 expressing cell lines. AED_{DMF5 01} and WT DMF5 T cells showed similar pattern of activation across an extensive range of biAb concentration. Technical replicates (n=3) (FIG. 11).

### Example 6: Motif Libraries Design, Generation and Sequencing

Deep mutational scanning (DMS) of DMF5 TCR combinatorial libraries (400 variants) were designed for each motif. Motif Libraries were generated by plasmid nicking mutagenesis. Briefly, the protocol relies on the presence of a single BbvCI restriction site for sequential targeting with Nt.BbvCl and Nb.BbvCI nickases, digestion of wild-type plasmid and plasmid re-synthesis using mutagenic oligonucleotides. Mutagenic oligonucleotides were designed using the QuikChange Primer Design online tool (Agilent). After nicking mutagenesis, mutated plasmids were transformed into 100 μl of chemically-competent E. coli DH5α cells (NEB, #C2987H) and plated on ampicillin (100 μg/mL) LB agar in Nunc BioAssay dishes (Sigma-Aldrich, #D4803). Serial dilutions of transformed cells were plated separately to quantify bacterial transformants. Plasmid libraries were purified from bacterial transformants using the ZymoPURE Plasmid miniprep Kit (Zymo Research, #D412). HDR templates were amplified from plasmid libraries by PCR and column-purified prior to transfection.

DMS library HDR templates and CDR3B gRNA were used to transfect 1×10⁶ lab modified Jurkat T cells. Firstly, cells FACS sorted for the CD3 surface expression. In the second round, cells were challenged separately with their cognate peptide (0.1 μg/mL) or blinatumomab (12 ng/ml) and both GFP− and GFP+ fractions were sorted (SEL1). Genomic DNA from all sorted populations was extracted via PureLink Genomic DNA Mini Kit (ThermoFisher, #K182002). Regions of interest were PCR amplified with added TruSeq adapters for 300-PE v3 (600 cycles). MiSeq sequencing was performed in the Genomics Facility Basel.

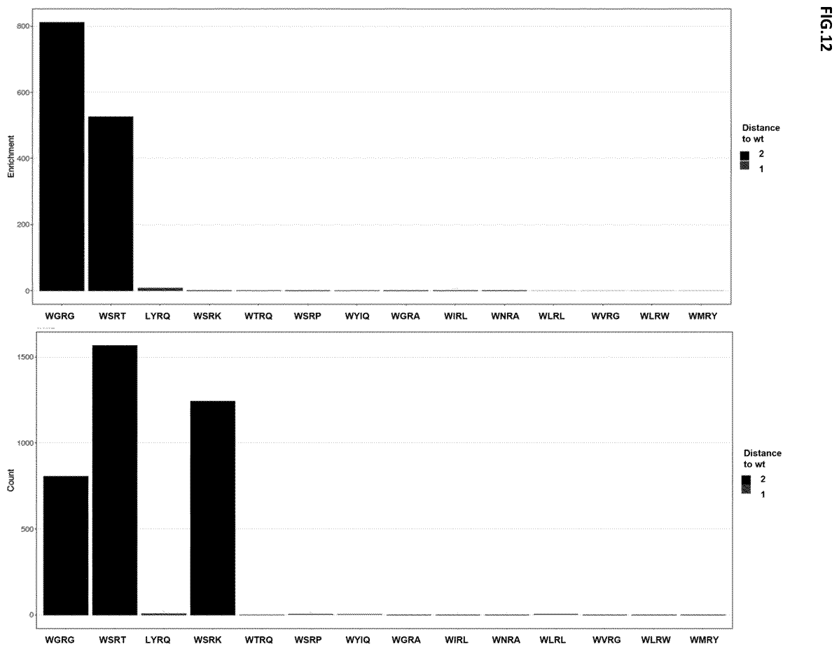

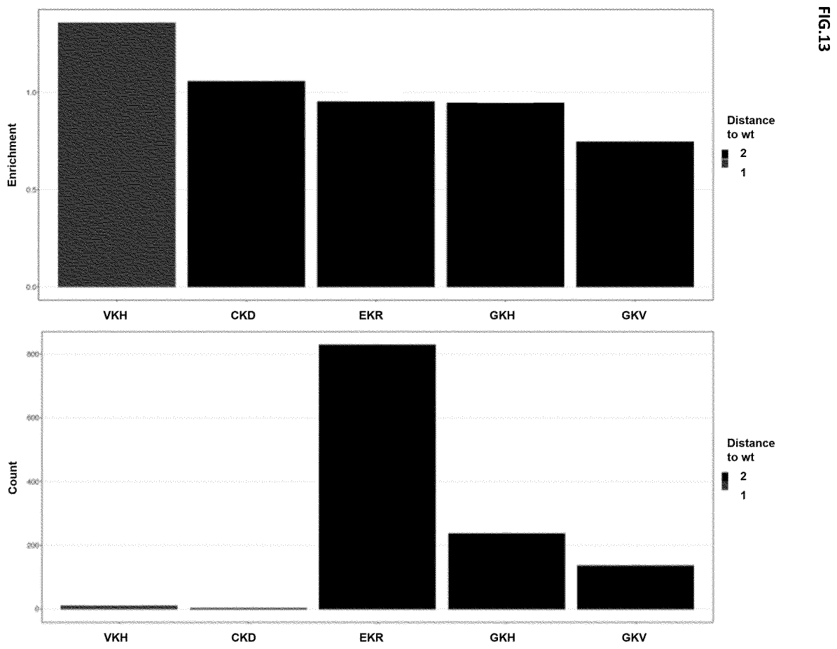

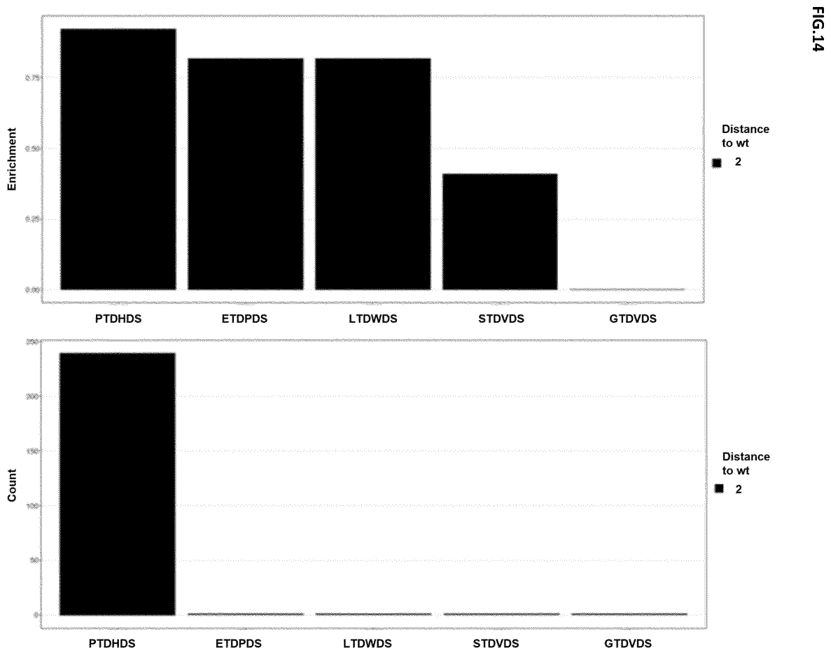

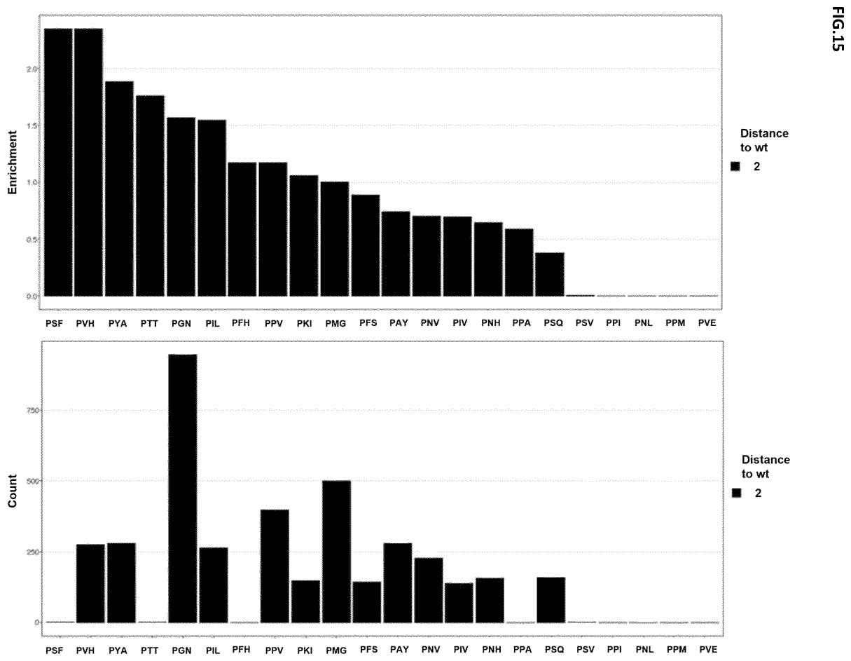

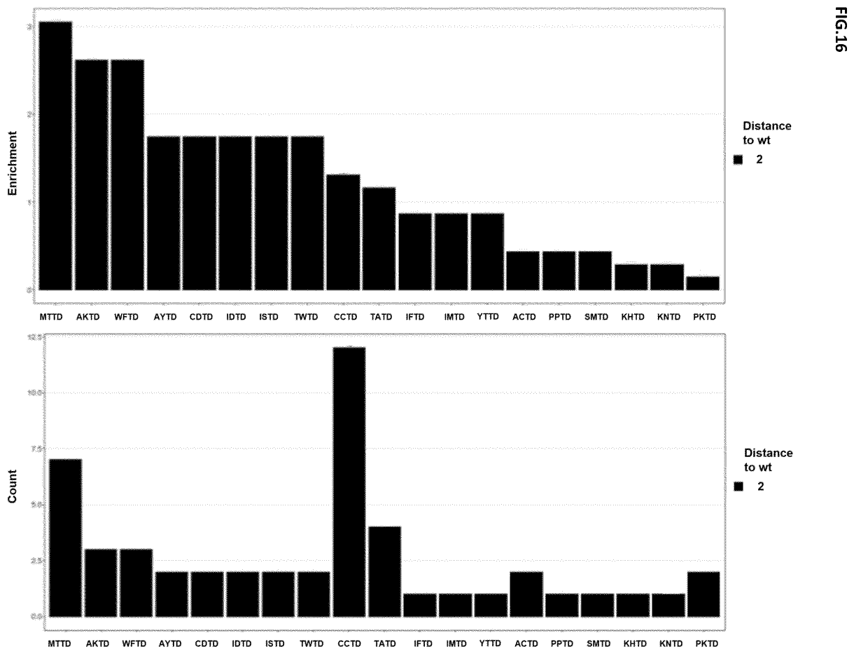

Fold enrichment and total count of motifs that were exclusively found in the bulk fraction (CD3⁺ fraction) and the blinatumomab fraction, but not in the peptide fraction are shown for different motifs in FIGS. 12-16.

1. A T cell comprising an engineered TCR-CD3 complex, wherein
c) binding of the engineered TCR-CD3 complex by a CD3 agonist results in a similar level of T cell activation compared to a T cell comprising a non-engineered TCR-CD3 complex; and
d) binding of the engineered TCR-CD3 complex by a cognate peptide-MHC complex results in a reduced level of T cell activation compared to a T cell comprising a non-engineered TCR-CD3 complex.

2. The T cell according to claim 1, wherein the engineered TCR-CD3 complex comprises at least one mutation in a TCR alpha and/or beta chain.

3. The T cell according to claim 2, wherein the at least one mutation in the TCR alpha and/or beta chain is located outside of a complementary determining region (CDR).

4. The T cell according to claim 2 or 3, wherein at least one mutation in the TCR alpha and/or beta chain is located at the interface between the TCR alpha and beta chain.

5. The T cell according to any one of claims 2-4, wherein the at least one mutation in the TCR alpha and/or beta chain results in reduced TCR alpha and beta association.

6. The T cell according to any one of claims 2-5, wherein the at least one mutation in the TCR alpha and/or beta chain has been introduced in the sequence motif WYRQ (IMGT position 41-44), FG₁xG₂T (IMGT position 118-122), VxP (IMGT position 126-128), PDP (position 4-6 of TCR alpha constant region (SEQ ID NO:25)), TDFDS (position 24-29 of TCR alpha constant region (SEQ ID NO:25)) and/or FETDxNLN (position 103-110 of TCR alpha constant region (SEQ ID NO:25)), wherein x is an undefined amino acid.

7. The T cell according to claim 6, wherein at least one of the amino acid residues G₁ and/or G₂ in the motif FG₁xG₂T (IMGT position 118-122) has been replaced with another amino acid.

8. The T cell according to claim 6 or 7, wherein the motif FG₁xG₂T in the TCR alpha and/or beta chain has been replaced with the sequence FEQWT.

9. The T cell according to any one of claims 2-8, wherein the at least one mutation in the engineered TCR-CD3 complex has been introduced:
a) at position W41, Y/F42, R/Q43, Q44, F118, G119, G121, T/S122, V126 and/or P128 in the variable domain of the TCR alpha chain (according to IMGT numbering); and/or
b) at position W41, Y/F42, R/Q43, Q44, F118, G119, G121, T/S122, T125 and/or V/L/T127 in the variable domain of the TCR beta chain (according to IMGT numbering); and/or
c) at position P4, D5, P6, F24, T25, D26, F27, D28, S29, F103, E104, T105, D106, N108, L109, N110 in the TCR alpha constant region (SEQ ID NO:25).

10. The T cell according to any one of claims 2-9, wherein the T cell is a human T cell.

11. The T cell according to any one of claims 2-10, wherein the TCR alpha and/or beta chain further comprise an affinity tag.

12. The T cell according to claim 11, wherein the affinity tag is inserted between the signal peptide and the coding sequence of the TCR alpha and/or beta chain.

13. The T cell according to any one of claims 1-12, wherein the CD3 agonist is an anti-CD3 antibody or a fragment thereof.

14. The T cell according to claim 13, wherein the anti-CD3 antibody is a bispecific antibody.

15. The T cell according to claim 14, wherein the bispecific antibody is blinatumomab.

16. The T cell according to claim 13, wherein the anti-CD3 antibody fragment is comprised in a fusion protein.

17. The T cell according to claim 16, wherein the fusion protein further comprises a fragment of a T cell receptor.

18. The T cell according to any one of claims 1-17, wherein the level of T cell activation is characterized by:
a) the secretion of interleukin-2 (IL-2); and/or
b) the secretion of interferon gamma (IFN-γ); and/or
c) the rate of T cell proliferation.

19. A T cell population comprising a plurality of T cells according to any one of claims 1-18.

20. A pharmaceutical composition comprising the T cell according to any one of claims 1-18 or a T cell population according to claim 19.

21. The pharmaceutical composition according to claim 20 further comprising a CD3 agonist.

22. The T cell according to any one of claims 1-18, the T cell population according to claim 19 or the pharmaceutical composition according to claim 20 or 21 for use as a medicament.

23. The T cell according to any one of claims 1-18, the T cell population according to claim 19 or the pharmaceutical composition according to claim 20 or 21 for use in the treatment of cancer.

24. The T cell according to any one of claims 1-18, the T cell population according to claim 19 or the pharmaceutical composition according to claim 20 or 21 for use in the treatment of a viral infection.

25. The T cell according to any one of claims 1-18, the T cell population according to claim 19 or the pharmaceutical composition according to claim 20 or 21 for use in the treatment of an autoimmune disease.

26. The T cell, the T cell population or the pharmaceutical composition for use according to any one of claims 22-25, wherein the human T cell has been obtained from a patient to be treated.

27. The T cell, the T cell population or the pharmaceutical composition for use according to any one of claims 22-25, wherein the human T cell has been obtained from a donor.

28. The T cell, the T cell population or the pharmaceutical composition for use according to any one of claims 22-27, wherein the human T cell is administered before, concomitantly or after a CD3 agonist.

29. A method for generating a T cell according to any one of claims 1-18, the method comprising the steps of:
i) introducing at least one mutation into a TCR alpha and/or beta chain of a T cell that has been obtained from a donor;
ii) obtaining a T cell comprising and engineered TCR-CD3 complex.

30. The method according to claim 29, wherein the at least one mutation in the TCR alpha and/or beta chain is introduced outside of a complementary determining region (CDR).

31. The method according to claim 29 or 30, wherein the at least one mutation in the TCR alpha and/or beta chain is introduced at the interface between the TCR alpha and beta chain.

32. The method according to any one of claims 29-31, wherein the at least one mutation in the TCR alpha and/or beta chain results in reduced TCR alpha and beta association.

33. The method according to any one of claims 29-32, wherein the at least one mutation in the TCR alpha and/or beta chain is introduced in the sequence motif WYRQ (IMGT position 41-44), FG₁xG₂T (IMGT position 118-122), VxP (IMGT position 126-128), PDP (position 4-6 of TCR alpha constant region (SEQ ID NO:25)), TDFDS (position 24-29 of TCR alpha constant region (SEQ ID NO:25)) and/or FETDxNLN (position 103-110 of TCR alpha constant region (SEQ ID NO:25)), wherein x is an undefined amino acid.

34. The method according to claim 33, wherein at least one of the amino acid residues G₁ and/or G₂ in the motif FG₁xG₂T (IMGT position 118-122) is replaced with another amino acid.

35. The method according to claim 33 or 34, wherein the motif FG₁xG₂T in the TCR alpha and/or beta chain is replaced with the sequence FEQWT.

36. The method according to any one of claims 29-35, wherein the at least one mutation in the TCR alpha and/or beta chain is introduced:
a) at position W41, Y/F42, R/Q43, Q44, F118, G119, G121, T/S122, V126 and/or P128 of the variable domain of the TCR alpha chain (according to IMGT numbering); and/or
b) at position W41, Y/F42, R/Q43, Q44, F118, G119, G121, T/S122, T125 and/or V/L/T127 of the variable domain of the TCR beta chain (according to IMGT numbering); and/or
c) at position P4, D5, P6, F24, T25, D26, F27, D28, S29, F103, E104, T105, D106, N108, L109, N110 of the TCR alpha constant region (SEQ ID NO:25).

37. The method according to any one of claims 29-36, wherein the T cell is a human T cell.

38. A method for generating a decoupled T cell receptor, the method comprising the steps of:
i) introducing at least one mutation into a TCR alpha and/or beta chain of a T cell receptor,
wherein the at least one mutation in the TCR alpha and/or beta chain is introduced in the sequence motif WYRQ (IMGT position 41-44), FG₁xG₂T (IMGT position 118-122), VxP (IMGT position 126-128), PDP (position 4-6 of TCR alpha constant region (SEQ ID NO:25)), TDFDS (position 24-29 of TCR alpha constant region (SEQ ID NO:25)) and/or FETDxNLN (position 103-110 of TCR alpha constant region (SEQ ID NO:25)), wherein x is an undefined amino; and/or
wherein the at least one mutation in the TCR alpha and/or beta chain is introduced:
a) at position W41, Y/F42, R/Q43, Q44, F118, G119, G121, T/S122, V126 and/or P128 of the variable domain of the TCR alpha chain (according to IMGT numbering); and/or
b) at position W41, Y/F42, R/Q43, Q44, F118, G119, G121, T/S122, T125 and/or V/L/T127 of the variable domain of the TCR beta chain (according to IMGT numbering); and/or
c) at position P4, D5, P6, F24, T25, D26, F27, D28, S29, F103, E104, T105, D106, N108, L109, N110 of the TCR alpha constant region (SEQ ID NO:25); and
ii) obtaining a decoupled T cell receptor.

39. The method according to any one of claims 29-38, wherein the at least one mutation is introduced into the TCR alpha and/or beta chain by genome editing.

40. The method according to claim 39, wherein the genome editing step involves the use of a CRISPR-Cas system.

41. The method according to any one of claims 29-40 further comprising a step of introducing a nucleic acid encoding an affinity tag into the TCR alpha and/or beta chain.


SEQUENCE LISTING (186 sequences)

SEQ ID NO: 1 - protein, 110 aa, Homo sapiens
  REGION 1..110: note = DMF5 alpha chain variable region
KEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPN

SEQ ID NO: 2 - protein, 113 aa, Homo sapiens
  REGION 1..113: note = DMF5 beta chain variable region
IAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVE

SEQ ID NO: 3 - protein, 114 aa, Homo sapiens
  REGION 1..114: note = IG4 alpha chain variable region
QEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPY

SEQ ID NO: 4 - protein, 112 aa, Homo sapiens
  REGION 1..112: note = IG4 beta chain variable region
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLE

SEQ ID NO: 5 - protein, 115 aa, synthetic construct
  REGION 1..115: note = a3a alpha chain variable region
AQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPN

SEQ ID NO: 6 - protein, 112 aa, synthetic construct
  REGION 1..112: note = a3a beta chain variable region
AGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTE

SEQ ID NO: 7 - protein, 116 aa, Homo sapiens
  REGION 1..116: note = 1406 alpha chain variable region
EFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYGEDDKIIFGKGTRLHILP

SEQ ID NO: 8 - protein, 113 aa, Homo sapiens
  REGION 1..113: note = 1406 beta chain variable region
LMEADIYQTPRYLVIGTGKKITLECSQTMGHDKMYWYQQDPGMELHLIHYSYGVNSTEKGDLSSESTVSRIRTEHFPLTLESARPSHTSQYLCASRRGPYEQYFGPGTRLTVT

SEQ ID NO: 9 - protein, 5 aa, synthetic construct
  REGION 1..5: note = AED_DMF5_01
FEQWT

SEQ ID NO: 10 - protein, 5 aa, synthetic construct
  REGION 1..5: note = AED_DMF5_02
FWQST

SEQ ID NO: 11 - protein, 5 aa, synthetic construct
  REGION 1..5: note = AED_DMF5_03
FHQAT

SEQ ID NO: 12 - protein, 5 aa, synthetic construct
  REGION 1..5: note = AED_DMF5_04
FYQLT

SEQ ID NO: 13 - protein, 5 aa, synthetic construct
  REGION 1..5: note = AED _DMF5_05
FHQVT

SEQ ID NO: 14 - protein, 5 aa, synthetic construct
  REGION 1..5: note = AED_IG4_01
FSQVT

SEQ ID NO: 15 - protein, 5 aa, synthetic construct
  REGION 1..5: note = AED_IG4_02
FVQLT

SEQ ID NO: 16 - protein, 5 aa, synthetic construct
  REGION 1..5: note = AED_IG4_03
FLQGT

SEQ ID NO: 17 - protein, 5 aa, synthetic construct
  REGION 1..5: note = AED_IG4_04
FRQAT

SEQ ID NO: 18 - protein, 5 aa, synthetic construct
  REGION 1..5: note = AED_IG4_05
FKQET

SEQ ID NO: 19 - protein, 5 aa, synthetic construct
  REGION 1..5: note = AED_a3a_01
FWQST

SEQ ID NO: 20 - protein, 5 aa, synthetic construct
  REGION 1..5: note = AED_a3a_02
FVQLT

SEQ ID NO: 21 - protein, 5 aa, synthetic construct
  REGION 1..5: note = AED_a3a_03
FIQST

SEQ ID NO: 22 - protein, 5 aa, synthetic construct
  REGION 1..5: note = AED_1406_01
FSQVT

SEQ ID NO: 23 - protein, 5 aa, synthetic construct
  REGION 1..5: note = AED_1406_02
FRQAT

SEQ ID NO: 24 - protein, 5 aa, synthetic construct
  REGION 1..5: note = AED_1406_03
FIQST

SEQ ID NO: 25 - protein, 140 aa, Homo sapiens
  REGION 1..140: note = TCR alpha chain constant region
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - protein, 129 aa, Homo sapiens
  REGION 1..129: note = TCR beta chain constant region
DLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 27 - protein, 8 aa, synthetic construct
  REGION 1..8: note = Mutated aCPM motif
FATADALN

SEQ ID NO: 28 - protein, 7 aa, synthetic construct
  REGION 1..7: note = Mutated aCPM motif
GGGSGSG

SEQ ID NO: 29 - RNA, 23 nt, synthetic construct
  misc_feature 1..23: note = CDR3-beta gRNA
  source 1..23: mol_type = other RNA
tcgacctgttcggctaactatgg

SEQ ID NO: 30 - protein, 20 aa, synthetic construct
  REGION 1..20: note = FGxGT motif in TRAJ01
YESITSQLQFGKGTRVSTSP

SEQ ID NO: 31 - protein, 21 aa, synthetic construct
  REGION 1..21: note = FGxGT motif in TRAJ02
NTGGTIDKLTFGKGTHVFIIS

SEQ ID NO: 32 - protein, 20 aa, synthetic construct
  REGION 1..20: note = FGxGT motif in TRAJ03
GYSSASKIIFGSGTRLSIRP

SEQ ID NO: 33 - protein, 20 aa, synthetic construct
  REGION 1..20: note = FGxGT motif in TRAJ04
FSGGYNKLIFGAGTRLAVHP

SEQ ID NO: 34 - protein, 19 aa, synthetic construct
  REGION 1..19: note = FGxGT motif in TRAJ05
DTGRRALTFGSGTRLQVQP

SEQ ID NO: 35 - protein, 20 aa, synthetic construct
  REGION 1..20: note = FGxGT motif in TRAJ06
ASGGSYIPTFGRGTSLIVHP

SEQ ID NO: 36 - protein, 19 aa, synthetic construct
  REGION 1..19: note = FGxGT motif in TRAJ07
DYGNNRLAFGKGNQVVVIP

SEQ ID NO: 37 - protein, 19 aa, synthetic construct
  REGION 1..19: note = FGxGT motif in TRAJ08
NTGFQKLVFGTGTRLLVSP

SEQ ID NO: 38 - protein, 20 aa, synthetic construct
  REGION 1..20: note = FGxGT motif in TRAJ09
GNTGGFKTIFGAGTRLFVKA

SEQ ID NO: 39 - protein, 21 aa, synthetic construct
  REGION 1..21: note = FGxGT motif in TRAJ10
ILTGGGNKLTFGTGTQLKVEL

SEQ ID NO: 40 - protein, 19 aa, synthetic construct
  REGION 1..19: note = FGxGT motif in TRAJ11
NSGYSTLTFGKGTMLLVSP

SEQ ID NO: 41 - protein, 19 aa, synthetic construct
  REGION 1..19: note = FGxGT motif in TRAJ12
MDSSYKLIFGSGTRLLVRP

SEQ ID NO: 42 - protein, 20 aa, synthetic construct
  REGION 1..20: note = FGxGT motif in TRAJ13-01
NSGGYQKVTFGIGTKLQVIP

SEQ ID NO: 43 - protein, 20 aa, synthetic construct
  REGION 1..20: note = FGxGT motif in TRAJ13-02
NSGGYQKVTFGTGTKLQVIP

SEQ ID NO: 44 - protein, 17 aa, synthetic construct
  REGION 1..17: note = FGxGT motif in TRAJ14
IYSTFIFGSGTRLSVKP

SEQ ID NO: 45 - protein, 19 aa, synthetic construct
  REGION 1..19: note = FGxGT motif in TRAJ15-01
NQAGTALIFGKGTTLSVSS

SEQ ID NO: 46 - protein, 19 aa, synthetic construct
  REGION 1..19: note = FGxGT motif in TRAJ15-02
NQAGTALIFGKGTHLSVSS

SEQ ID NO: 47 - protein, 19 aa, synthetic construct
  REGION 1..19: note = FGxGT motif in TRAJ16
FSDGQKLLFARGTMLKVDL

SEQ ID NO: 48 - protein, 18 aa, synthetic construct
  REGION 1..18: note = FGxGT motif in TRAJ47-02
EYGNKLVFGAGTILRVKS

SEQ ID NO: 49 - protein, 20 aa, synthetic construct
  REGION 1..20: note = FGxGT motif in TRAJ48
SNFGNEKLTFGTGTRLTIIP

SEQ ID NO: 50 - protein, 18 aa, synthetic construct
  REGION 1..18: note = FGxGT motif in TRAJ49
NTGNQFYFGTGTSLTVIP

SEQ ID NO: 51 - protein, 250 aa, Homo sapiens
  REGION 1..250: note = DMF5 alpha chain
KEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 52 - protein, 242 aa, Homo sapiens
  REGION 1..242: note = DMF5 beta chain
IAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 53 - protein, 254 aa, Homo sapiens
  REGION 1..254: note = IG4 alpha chain
QEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - protein, 241 aa, Homo sapiens
  REGION 1..241: note = IG4 beta chain
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 55 - protein, 255 aa, synthetic construct
  REGION 1..255: note = A3A alpha chain
AQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 56 - protein, 241 aa, synthetic construct
  REGION 1..241: note = A3A beta chain
AGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 57 - protein, 5 aa, synthetic construct
  REGION 1..5: note = DMF5 TCR FGxGT motif
FGQGT

SEQ ID NO: 58 - protein, 5 aa, synthetic construct
  REGION 1..5: note = IG4 TCR FGxGT motif
FGKGT

SEQ ID NO: 59 - protein, 5 aa, synthetic construct
  REGION 1..5: note = A3a TCR FGxGT motif
FGKGT

SEQ ID NO: 60 - protein, 9 aa, synthetic construct
  REGION 1..9: note = MART-1 peptide antigen 1
AAGIGILTV

SEQ ID NO: 61 - protein, 10 aa, synthetic construct
  REGION 1..10: note = MART-1 peptide antigen 2
EAAGIGILTV

SEQ ID NO: 62 - protein, 10 aa, synthetic construct
  REGION 1..10: note = MART-1 peptide antigen 3
ELAGIGILTV

SEQ ID NO: 63 - protein, 9 aa, synthetic construct
  REGION 1..9: note = NY-ESO 1 peptide antigen
SLLMWITQC

SEQ ID NO: 64 - protein, 9 aa, synthetic construct
  REGION 1..9: note = MAGE-A3 peptide antigen
EVDPIGHLY

SEQ ID NO: 65 - protein, 10 aa, synthetic construct
  REGION 1..10: note = HCV peptide antigen
KLVALGINAV

SEQ ID NO: 66 - protein, 10 aa, synthetic construct
  REGION 1..10: note = DMF5 CDR3a
AVNFGGGKLI

SEQ ID NO: 67 - protein, 13 aa, synthetic construct
  REGION 1..13: note = 1G4 CDR3a
AVRPTSGGSYIPT

SEQ ID NO: 68 - protein, 12 aa, synthetic construct
  REGION 1..12: note = A3a CDR3a
AVRPGGAGPFFW

SEQ ID NO: 69 - protein, 9 aa, synthetic construct
  REGION 1..9: note = 1406 CDR3a
AYGEDDKII

SEQ ID NO: 70 - protein, 5 aa, synthetic construct
  REGION 1..5: note = DMF5 FGxGT variant
FVQRT

SEQ ID NO: 71 - protein, 5 aa, synthetic construct
  REGION 1..5: note = DMF5 FGxGT variant
FRQRT

SEQ ID NO: 72 - protein, 5 aa, synthetic construct
  REGION 1..5: note = DMF5 FGxGT variant
FCQAT

SEQ ID NO: 73 - protein, 5 aa, synthetic construct
  REGION 1..5: note = DMF5 FGxGT variant
FVQYT

SEQ ID NO: 74 - protein, 5 aa, synthetic construct
  REGION 1..5: note = IG4 FGxGT variant
FSQRT

SEQ ID NO: 75 - protein, 5 aa, synthetic construct
  REGION 1..5: note = IG4 FGxGT variant
FAQGT

SEQ ID NO: 76 - protein, 5 aa, synthetic construct
  REGION 1..5: note = IG4 FGxGT variant
FVQWT

SEQ ID NO: 77 - protein, 5 aa, synthetic construct
  REGION 1..5: note = A3a FGxGT variant
FSQGT

SEQ ID NO: 78 - protein, 5 aa, synthetic construct
  REGION 1..5: note = A3a FGxGT variant
FAQGT

SEQ ID NO: 79 - protein, 5 aa, synthetic construct
  REGION 1..5: note = A3a FGxGT variant
FRQAT

SEQ ID NO: 80 - protein, 5 aa, synthetic construct
  REGION 1..5: note = A3a FGxGT variant
FYQGT

SEQ ID NO: 81 - protein, 5 aa, synthetic construct
  REGION 1..5: note = A3a FGxGT variant
FTQMT

SEQ ID NO: 82 - protein, 256 aa, synthetic construct
  REGION 1..256: note = 1406 alpha chain
EFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYGEDDKIIFGKGTRLHILPIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 83 - protein, 242 aa, synthetic construct
  REGION 1..242: note = 1406 beta chain
LMEADIYQTPRYLVIGTGKKITLECSQTMGHDKMYWYQQDPGMELHLIHYSYGVNSTEKGDLSSESTVSRIRTEHFPLTLESARPSHTSQYLCASRRGPYEQYFGPGTRLTVTDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 84 - DNA, 16 bp, synthetic construct
  misc_feature 1..16: note = Fwd primer F1
  source 1..16: mol_type = other DNA
gcatggatcccaatgc

SEQ ID NO: 85 - DNA, 24 bp, synthetic construct
  misc_feature 1..24: note = Rev primer R1
  source 1..24: mol_type = other DNA
ttttatctgtcatggccgtgaccg

SEQ ID NO: 86 - RNA, 20 nt, synthetic construct
  misc_feature 1..20: note = TRAC locus sgRNA
  source 1..20: mol_type = other RNA
agagtttgatcctggctcag

SEQ ID NO: 87 - protein, 4 aa, synthetic construct
  REGION 1..4: note = WYRQ Motif
WYRQ

SEQ ID NO: 88 - protein, 5 aa, synthetic construct
  REGION 1..5: note = FGxGT motif
  VARIANT 3: note = Xaa can be any amino acid
FGXGT

SEQ ID NO: 89 - protein, 8 aa, synthetic construct
  REGION 1..8: note = FETDxNLN motif
  VARIANT 5: note = Xaa can be any amino acid
FETDXNLN

SEQ ID NO: 90 - protein, 6 aa, synthetic construct
  REGION 1..6: note = FTDFDS motif
FTDFDS

SEQ ID NO: 91 - protein, 5 aa, synthetic construct
  REGION 1..5: note = FGEGS motif
FGEGS

SEQ ID NO: 92 - protein, 5 aa, synthetic construct
  REGION 1..5: note = FGRGT motif
FGRGT

SEQ ID NO: 93 - protein, 5 aa, synthetic construct
  REGION 1..5: note = FGPGT motif
FGPGT

SEQ ID NO: 94 - protein, 4 aa, synthetic construct
  REGION 1..4: note = WFRQ
WFRQ

SEQ ID NO: 95 - protein, 4 aa, synthetic construct
  REGION 1..4: note = WYQQ motif
WYQQ

SEQ ID NO: 96 - protein, 4 aa, synthetic construct
  REGION 1..4: note = WYKQ motif
WYKQ

SEQ ID NO: 97 - protein, 4 aa, synthetic construct
  REGION 1..4: note = WYQQ motif
WYQQ

SEQ ID NO: 98 - protein, 12 aa, synthetic construct
  REGION 1..12: note = TRAJ01 01
QFGKGTRVSTSP

SEQ ID NO: 99 - protein, 12 aa, synthetic construct
  REGION 1..12: note = TRAJ02 01
TFGKGTHVFIIS

SEQ ID NO: 100 - protein, 12 aa, synthetic construct
  REGION 1..12: note = TRAJ03 01
IFGSGTRLSIRP

SEQ ID NO: 101 - protein, 12 aa, synthetic construct
  REGION 1..12: note = TRAJ04 01
IFGAGTRLAVHP

SEQ ID NO: 102 - protein, 12 aa, synthetic construct
  REGION 1..12: note = TRAJ05 01
TFGSGTRLQVQP

SEQ ID NO: 103 - protein, 12 aa, synthetic construct
  REGION 1..12: note = TRAJ06 01
TFGRGTSLIVHP

SEQ ID NO: 104 - protein, 12 aa, synthetic construct
  REGION 1..12: note = TRAJ07 01
AFGKGNQVVVIP

SEQ ID NO: 105 - protein, 12 aa, synthetic construct
  REGION 1..12: note = TRAJ08 01
VFGTGTRLLVSP

SEQ ID NO: 106 - protein, 12 aa, synthetic construct
  REGION 1..12: note = TRAJ09 01
IFGAGTRLFVKA

SEQ ID NO: 107 - protein, 12 aa, synthetic construct
  REGION 1..12: note = TRAJ10 01
TFGTGTQLKVEL

SEQ ID NO: 108 - protein, 12 aa, synthetic construct
  REGION 1..12: note = TRAJ11 01
TFGKGTMLLVSP

SEQ ID NO: 109 - protein, 12 aa, synthetic construct
  REGION 1..12: note = TRAJ12 01
IFGSGTRLLVRP

SEQ ID NO: 110 - protein, 12 aa, synthetic construct
  REGION 1..12: note = TRAJ13 01
TFGIGTKLQVIP

SEQ ID NO: 111 - protein, 12 aa, synthetic construct
  REGION 1..12: note = TRAJ13 02
TFGTGTKLQVIP

SEQ ID NO: 112 - protein, 12 aa, synthetic construct
  REGION 1..12: note = TRAJ14 01
IFGSGTRLSVKP

SEQ ID NO: 113 - protein, 12 aa, synthetic construct
  REGION 1..12: note = TRAJ15 01
IFGKGTTLSVSS

SEQ ID NO: 114 - protein, 12 aa, synthetic construct
  REGION 1..12: note = TRAJ15 02
IFGKGTHLSVSS

SEQ ID NO: 115 - protein, 12 aa, synthetic construct
  REGION 1..12: note = TRAJ16 01
LFARGTMLKVDL

SEQ ID NO: 116 - protein, 12 aa, synthetic construct
  REGION 1..12: note = TRAJ47 02
VFGAGTILRVKS

SEQ ID NO: 117 - protein, 12 aa, synthetic construct
  REGION 1..12: note = TRAJ48 01
TFGTGTRLTIIP

SEQ ID NO: 118 - protein, 12 aa, synthetic construct
  REGION 1..12: note = TRAJ49 01
YFGTGTSLTVIP

SEQ ID NO: 119 - protein, 203 aa, synthetic construct [sequence omitted]

SEQ ID NO: 120 - protein, 241 aa, synthetic construct [sequence omitted]

SEQ ID NO: 121 - protein, 197 aa, synthetic construct
  REGION 1..197: note = A3A alpha chain
AQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIP

SEQ ID NO: 122 - protein, 241 aa, synthetic construct
  REGION 1..241: note = A3A beta chain
AGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 123 - protein, 5 aa, synthetic construct
FWQST

SEQ ID NO: 124 - protein, 5 aa, synthetic construct
FHQAT

SEQ ID NO: 125 - protein, 5 aa, synthetic construct
FYQLT

SEQ ID NO: 126 - protein, 5 aa, synthetic construct
FHQVT

SEQ ID NO: 127 - protein, 5 aa, synthetic construct
FVQRT

SEQ ID NO: 128 - protein, 5 aa, synthetic construct
FRQRT

SEQ ID NO: 129 - protein, 5 aa, synthetic construct
FCQAT

SEQ ID NO: 130 - protein, 5 aa, synthetic construct
FVQYT

SEQ ID NO: 131 - protein, 5 aa, synthetic construct
FLQGT

SEQ ID NO: 132 - protein, 5 aa, synthetic construct
FRQAT

SEQ ID NO: 133 - protein, 5 aa, synthetic construct
FVQLT

SEQ ID NO: 134 - protein, 5 aa, synthetic construct
FSQVT

SEQ ID NO: 135 - protein, 5 aa, synthetic construct
FKQET

SEQ ID NO: 136 - protein, 5 aa, synthetic construct
FSQRT

SEQ ID NO: 137 - protein, 5 aa, synthetic construct
FAQGT

SEQ ID NO: 138 - protein, 5 aa, unidentified
FVQWT

SEQ ID NO: 139 - protein, 5 aa, synthetic construct
FVQLT

SEQ ID NO: 140 - protein, 5 aa, synthetic construct
FWQST

SEQ ID NO: 141 - protein, 5 aa, synthetic construct
FIQST

SEQ ID NO: 142 - protein, 5 aa, synthetic construct
FSQGT

SEQ ID NO: 143 - protein, 5 aa, synthetic construct
FAQGT

SEQ ID NO: 144 - protein, 5 aa, synthetic construct
FRQAT

SEQ ID NO: 145 - protein, 5 aa, synthetic construct
FYQGT

SEQ ID NO: 146 - protein, 5 aa, synthetic construct
FTQMT

SEQ ID NO: 147 - protein, 8 aa, synthetic construct
FATADALN

SEQ ID NO: 148 - protein, 7 aa, synthetic construct
GGGSGSG

SEQ ID NO: 149 - protein, 4 aa, synthetic construct
WGRG

SEQ ID NO: 150 - protein, 4 aa, synthetic construct
WSRT

SEQ ID NO: 151 - protein, 4 aa, synthetic construct
LYRQ

SEQ ID NO: 152 - protein, 4 aa, synthetic construct
WSRK

SEQ ID NO: 153 - protein, 4 aa, synthetic construct
WTRQ

SEQ ID NO: 154 - protein, 4 aa, synthetic construct
WSRP

SEQ ID NO: 155 - protein, 4 aa, synthetic construct
WYIQ

SEQ ID NO: 156 - protein, 4 aa, synthetic construct
WGRA

SEQ ID NO: 157 - protein, 4 aa, synthetic construct
WIRL

SEQ ID NO: 158 - protein, 4 aa, synthetic construct
WNRA

SEQ ID NO: 159 - protein, 4 aa, synthetic construct
WLRL

SEQ ID NO: 160 - protein, 4 aa, synthetic construct
WVRG

SEQ ID NO: 161 - protein, 4 aa, synthetic construct
WLRW

SEQ ID NO: 162 - protein, 4 aa, synthetic construct
WMRY

SEQ ID NO: 163 - protein, 6 aa, synthetic construct
PTDHDS

SEQ ID NO: 164 - protein, 6 aa, synthetic construct
ETDPDS

SEQ ID NO: 165 - protein, 6 aa, synthetic construct
LTDWDS

SEQ ID NO: 166 - protein, 6 aa, synthetic construct
STDVDS

SEQ ID NO: 167 - protein, 6 aa, synthetic construct
GTDVDS

SEQ ID NO: 168 - protein, 4 aa, synthetic construct
MTTD

SEQ ID NO: 169 - protein, 4 aa, synthetic construct
AKTD

SEQ ID NO: 170 - protein, 4 aa, synthetic construct
WFTD

SEQ ID NO: 171 - protein, 4 aa, synthetic construct
AYTD

SEQ ID NO: 172 - protein, 4 aa, synthetic construct
CDTD

SEQ ID NO: 173 - protein, 4 aa, synthetic construct
IDTD

SEQ ID NO: 174 - protein, 4 aa, synthetic construct
ISTD

SEQ ID NO: 175 - protein, 4 aa, synthetic construct
TWTD

SEQ ID NO: 176 - protein, 4 aa, synthetic construct
CCTD

SEQ ID NO: 177 - protein, 4 aa, synthetic construct
TATD

SEQ ID NO: 178 - protein, 4 aa, synthetic construct
IFTD

SEQ ID NO: 179 - protein, 4 aa, synthetic construct
IMTD

SEQ ID NO: 180 - protein, 4 aa, synthetic construct
YTTD

SEQ ID NO: 181 - protein, 4 aa, synthetic construct
ACTD

SEQ ID NO: 182 - protein, 4 aa, synthetic construct
PPTD

SEQ ID NO: 183 - protein, 4 aa, synthetic construct
SMTD

SEQ ID NO: 184 - protein, 4 aa, synthetic construct
KHTD

SEQ ID NO: 185 - protein, 4 aa, synthetic construct
KNTD

SEQ ID NO: 186 - protein, 4 aa, synthetic construct
PKTD